(19) United States
(12) Patent Application Publication
(10) Pub. No.: US 2006/0154930 A1
(43) Pub. Date: Jul. 13, 2006

(54) Substituted amino heterocycles as vr-1 antagonists for treating pain

(76) Inventors: Rebecca Elizabeth Brown, Cambridge (GB); Frank Burkamp, Bishops Stortford (GB); Victoria Alexandra Doughty, Stansted (GB); Stephen Robert Fischer, Bishops Stortford (GB); Gregory John Hollingworth, Brentwood (GB); A. Brian Jones, Saffron Walden (GB); Timothy Jason Sparey, London (GB)

Correspondence Address: MERCK AND CO., INC, P O BOX 2000, RAHWAY, NJ, 07065-0907, US

(21) Appl. No.: 10/545,877

(22) PCT Filed: Feb. 20, 2004
(86) PCT No.: PCT/GB04/00702

§ 371 Date: Aug. 17, 2005

(30) Foreign Application Priority Data
Feb. 20, 2003 (GB) 0303910.4

(51) Int. Cl. C07D 487/04; A61K 31/503

(52) U.S. Cl. 514/248; 544/236

(57) ABSTRACT

The present invention provides compounds of formula I: in which: one of T¹ and T⁴ is N and the other is C; T² and T³ are independently N or C(CH₂)ₙR²; X, Y and Z are independently N or C(CH₂)ₙR³; R¹ is Ar¹ or R¹ is C₁₋₆alkyl optionally substituted with one or two groups Ar1; Ar1 is an optionally substituted cyclohexyl, piperidinyl, piperazinyl, morpholinyl, adamantyl, phenyl, naphthyl, a six membered heteroaromatic ring containing one, two or three nitrogen atoms, a five-membered heteroaromatic ring containing one, two, three or four heteroatoms chosen from O, N and S, at most one O or S atom being present, or a nine- or ten-membered bicyclic heteroaromatic ring in which phenyl or a six-membered heteroaromatic ring as defined above is fused to a six- or five-membered heteroaromatic ring as defined above; Ar is an optionally substituted phenyl, a six-membered heteroaromatic ring containing one, two or three nitrogen atoms or a five-membered heteroaromatic ring containing one, two, three or four heteroatoms chosen from O, N and S, at most one heteroatom being O or S, Ar being optionally substituted by one, two or three groups chosen from halogen, CF₃, OCF₃, C₁₋₆-alkyl, C₂₋₆alkenyl, C₂₋₆alkynyl, nitro, cyano, isonitrile, hydroxy, C₁₋₆alkoxy, C₁₋₆alkylthio, —NR⁶R⁷, —CONR⁶R⁷, —COH, CO₂H, C₁₋₆alkoxycarbonyl, haloC₁₋₄alkyl, hydroxyC₁₋₆alkyl, aminoC₁₋₆alkyl, C₁₋₄alkylcarbonyl and a five-membered heteroaromatic ring containing one, two, three or four heteroatoms chosen from O, N and S, at most one heteroatom being O or S, optionally substituted by C₁₋₆alkyl, halogen, amino, hydroxy or cyano; or a pharmaceutically acceptable salt thereof as a VR-1 ligand; pharmaceutical compositions comprising it; its use in therapy; use of it in the manufacture of a medicament to treat pain; and methods of treating subjects suffering from pain.

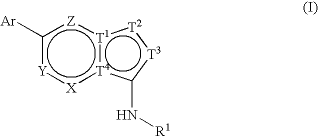

The present invention is concerned with substituted amino-heterocycles and pharmaceutically acceptable salts and prodrugs thereof which are useful as therapeutic compounds, particularly in the treatment of pain and other conditions ameliorated by the modulation of the function of the vanilloid-1 receptor (VR1).

The pharmacologically active ingredient of chilli peppers has been recognised for some time to be the phenolic amide capsaicin. The application of capsaicin to mucous membranes or when injected intradermally, causes intense burning-like pain in humans. The beneficial effects of topical administration of capsaicin as an analgesic is also well established. However, understanding of the underlying molecular pharmacology mediating these responses to capsaicin has been a more recent development.

The receptor for capsaicin, termed the vanilloid VR1 receptor, was cloned by Caterina and colleagues at UCSF in 1997 (Nature, 398:816, 1997). VR1 receptors are cation channels that are found on sensory nerves that innervate the skin, viscera, peripheral tissues and spinal cord. Activation of VR1 elicits action potentials in sensory fibres that ultimately generate the sensation of pain. Importantly VR1 receptor is activated not only by capsaicin but also by acidic pH and by noxious heat stimuli. It is also sensitized by a number of inflammatory mediators and thus appears to be a polymodal integrator of painful stimuli.

The prototypical VR1 antagonist is capsazepine (Walpole et al., J. Med. Chem., 37:1942, 1994)—VR1 IC₅₀ of 420 nM. A novel series of sub-micromolar antagonists has also been reported recently (Lee et al, Bioorg. Med. Chem., 9:1713, 2001), but these reports provide no evidence for in vivo efficacy. A much higher affinity antagonist has been derived from the ‘ultra-potent’ agonist resiniferatoxin. Iodo-resiniferatoxin (Wahl et al., Mol. Pharmacol, 59:9, 2001) is a nanomolar antagonist of VR1 but does not possess properties suitable for an oral pharmaceutical. This last is also true of the micromolar peptoid antagonists described by Garcia-Martinez (Proc. Natl. Acad. Sci., USA, 99:2374, 2002). Most recently International (PCT) patent publication No. WO 02/08221 has described a novel series of VR1 antagonists, which are stated to show efficacy in a number of animal models. We herein describe another novel series of VR1 modulators. These comprise predominantly VR1 antagonists but encompass VR1 partial antagonists and VR1 partial agonists. Such compounds have been shown to be efficacious in animal models of pain.

The present invention provides compounds of formula (I): 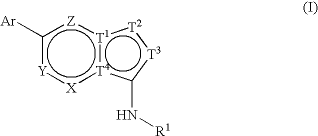
wherein:

one of T¹ and T⁴ is N and the other is C;

T² and T³ are independently N or C(CH₂)ₙR²;

X, Y and Z are independently N or C(CH₂)ₙR³;

R¹ is Ar¹ or R¹ is C₁₋₆alkyl optionally substituted with one or two groups Ar¹;

Ar¹ is cyclohexyl, piperidinyl, piperazinyl, morpholinyl, adamantyl, phenyl, naphthyl, a six-membered heteroaromatic ring containing one, two or three nitrogen atoms, a five-membered heteroaromatic ring containing one, two, three or four heteroatoms chosen from O, N and S, at most one O or S atom being present, or a nine- or ten-membered bicyclic heteroaromatic ring in which phenyl or a six-membered heteroaromatic ring as defined above is fused to a six- or five-membered heteroaromatic ring as defined above;

Ar¹ is optionally substituted by one, two or three groups chosen from halogen, hydroxy, cyano, nitro, isonitrile, CF₃, OCF₃, SF₅, C₁₋₆alkyl, C₂₋₆alkenyl C₂₋₆alkynyl, C₁₋₆alkoxy, C₁₋₆alkylthio, C₁₋₆alkylsulfinyl, C₁₋₆alkylsulfonyl, —NR⁶R⁷, CONR⁶R⁷, —COH, —CO₂H, C₁₋₆alkylcarbonyl, C₁₋₆alkoxycarbonyl, haloC₁₋₆alkyl, haloC₂₋₆alkenyl, hydroxyC₁₋₆ alkyl, aminoC₁₋₆alkyl, cyanoC₁₋₆alkyl, C₃₋₆cycloalkyl, hydroxyC₃₋₆cycloalkyl, aminoC₃₋₆cycloalkyl, haloC₃₋₆cycloalkyl, cyanoC₃₋₆cycloalkyl, haloC₁₋₆alkylcarbonyl, C₁₋₆alkoxycarbonylC₁₋₆alkyl, (halo)(hydroxy)C₁₋₆alkyl, (halo)(hydroxy)C₃₋₆cycloalkyl, phenyl and a five-membered heteroaromatic ring containing one, two or three heteroatoms, at most one O or S atom being present; wherein the phenyl and five-membered heteroaromatic ring are optionally substituted by C₁₋₆alkyl, halo, hydroxy or cyano; when two C₁₋₆alkyl groups substitute adjacent positions on Ar¹ then, together with the carbon atoms to which they are attached, they may form a partially saturated ring containing five or six carbon atoms; when two C₁₋₆alkoxy groups substitute adjacent positions on Ar¹ then, together with the carbon atoms to which they are attached, they may form a partially saturated five- or six-membered ring;

Ar is phenyl, a six-membered heteroaromatic ring containing one, two or three nitrogen atoms or a five-membered heteroaromatic ring containing one, two, three or four heteroatoms chosen from O, N and S, at most one heteroatom being O or S, Ar being optionally substituted by one, two or three groups chosen from halogen, CF₃, OCF₃, C₁₋₆alkyl, C₂₋₆alkenyl, C₂₋₆alkynyl, nitro, cyano, isonitrile, hydroxy, C₁₋₆alkoxy, C₁₋₆alkylthio, —NR⁶R⁷, —CONR⁶R⁷, —COH, CO₂H, C₁₋₆alkoxycarbonyl, haloC₁₋₆alkyl, hydroxyC₁₋₆alkyl, aminoC₁₋₆alkyl, C₁₋₆alkylcarbonyl and a five-membered heteroaromatic ring containing one, two, three or four heteroatoms chosen from O, N and S, at most one heteroatom being O or S, optionally substituted by C₁₋₆alkyl, halogen, amino, hydroxy or cyano;

R² and R³ are independently hydrogen, halogen, CF₃, OCF₃, C₁₋₆alkyl, C₂₋₆alkenyl, C₂₋₆alkynyl, nitro, cyano, isonitrile, hydroxy, C₁₋₆alkoxy, C₁₋₆alkylthio, —NR⁶R⁷, —CONR⁶R⁷, —COH, CO₂H, C₁₋₆alkoxycarbonyl, haloC₁₋₆alkyl, hydroxyC₁₋₆alkyl, aminoC₁₋₆alkyl, C₁₋₆alkylaminoC₁₋₆alkyl, di(C₁₋₆alkyl)aminoC₁₋₆alkyl, amido, piperidinyl, piperazinyl, C₃₋₆cycloalkyl, morpholinyl, phenyl, a six-membered heteroaromatic ring containing one, two or three nitrogen atoms or a five-membered heteroaromatic ring containing one, two, three or four heteroatoms chosen from O, N and S, at most one O or S atom being present, which phenyl, six-membered heteroaromatic ring and five-membered heteroaromatic ring are optionally substituted by haloC₁₋₆alkyl, C₁₋₆alkyl, hydroxy, halogen, amino or cyano;

R⁶ and R⁷ are independently hydrogen or C₁₋₆alkyl; when both R⁶ and R⁷ are C₁₋₆alkyl then, together with the nitrogen atom to which they are attached, they may form a five or six membered saturated nitrogen containing ring; and

n is zero, one, two or three;

or a pharmaceutically acceptable salt thereof.

In one embodiment, in the compounds of formula (I):

one of T¹ and T⁴ is N and the other is C;

T² and T³ are independently N or CR²;

X, Y and Z are independently N or CR³;

R¹ is Ar¹ or R¹ is C₁₋₆alkyl substituted with one or two groups Ar¹;

Ar¹ is phenyl, naphthyl, a six-membered heteroaromatic ring containing one, two or three nitrogen atoms, a five-membered ring containing one, two, three or four heteroatoms chosen from O, N and S, at most one O or S atom being present, or a nine- or ten-membered bicyclic heteroaromatic ring in which phenyl or a six-membered heteroaromatic ring as defined above is fused to a six- or five-membered heteroaromatic ring as defined above;

Ar¹ is optionally substituted by one, two or three groups chosen from halogen, hydroxy, cyano, nitro, nitrile, CF₃, OCF₃, C₁₋₆alkyl, C₂₋₆alkenyl, C₂₋₆alkynyl, C₁₋₆alkoxy, C₁₋₆alkylthio, —NR⁶R⁷, CONR⁶R⁷, —COH, —CO₂H, C₁₋₆alkoxycarbonyl, haloC₁₋₆alkyl, hydroxyC₁₋₆alkyl or aminoC₁₋₆alkyl; when two C₁₋₆alkyl groups substitute adjacent positions on Ar¹ then, together with the carbon atoms to which they are attached, they may form a partially saturated ring containing five or six carbon atoms; when two C₁₋₆alkoxy groups substitute adjacent positions on Ar¹ then, together with the carbon atoms to which they are attached, they may form a partially saturated five- or six-membered ring;

Ar is phenyl, a six-membered heteroaromatic ring containing one, two or three nitrogen atoms or a five-membered heteroaromatic ring containing one, two, three or four heteroatoms chosen from O, N and S, at most one heteroatom being O or S, Ar being optionally substituted by one, two or three groups chosen from halogen, CF₃, OCF₃, C₁₋₆alkyl, C₂₋₆alkenyl, C₂₋₆alkynyl, nitro, cyano, isonitrile, hydroxy, C₁₋₆alkoxy, C₁₋₆alkylthio, —NR⁶R⁷, —CONR⁶R⁷, —COH, CO₂H, C₁₋₆alkoxycarbonyl, haloC₁₋₆alkyl, hydroxyC₁₋₆alkyl and aminoC₁₋₆alkyl;

R² and R³ are independently hydrogen, halogen, CF₃, OCF₃, C₁₋₆alkyl, C₂₋₆alkenyl, C₂₋₆alkynyl, nitro, cyano, isonitrile, hydroxy, C₁₋₆alkoxy, C₁₋₆alkylthio, —NR⁶R⁷, —CONR⁶R⁷, —COH, CO₂H, C₁₋₆alkoxycarbonyl, haloC₁₋₆alkyl, hydroxyC₁₋₆alkyl or aminoC₁₋₆alkyl; and

R⁶ and R⁷ are independently hydrogen or C₁₋₆alkyl; when both R⁶ and R⁷ are C₁₋₆alkyl then, together with the nitrogen atom to which they are attached, they may form a five or six membered saturated nitrogen containing ring.

Preferred core structures are obtained when Y is C(CH₂)ₙR³. In this case it is generally preferred that: X is N, Z is C(CH₂)ₙR³, T⁴ is N, T² and T³ are N or T² is C(CH₂)ₙR² and T³ is N or T² is N and T³ is C(CH₂)ₙR²; or X and Z are C(CH₂)ₙR³ and T², T³ and T⁴ are N; or X is N, Z is C(CH₂)ₙR³, T³ is C(CH₂)ₙR² and T² and T¹ are N; or X, Z, T², T³ and T⁴ are N. Additional core structures include those where X and Z are N, T² and T⁴ are N and T³ is C(CH₂)ₙR²; or X and Z are C(CH₂)ₙR³, T² and T⁴ are N and T³ is C(CH₂)ₙR²; or X is C(CH₂)ₙR³, Z is N, T³ and T⁴ are N and T² is C(CH₂)ₙR².

Further exemplified core structures include those where Y is CR³. In this case it is generally preferred that: X is N, Z is CR³, T⁴ is N, T² and T³ are N or T² is CR² and T³ is N or T² is N and T³ is CR²; or X and Z are CR³ and T², T³ and T⁴ are N; or X is N, Z is CR³, T³ is CR² and T² and T¹ are N; or X, Z, T², T³ and T⁴ are N.

R¹ is preferably Ar¹ or C₁₋₄alkyl, especially C₁₋₂alkyl, substituted by one or two, preferably one, Ar¹ groups. In particular R¹ can be Ar¹. R¹ may be butyl. R¹ may be cyclohexyl, piperidinyl or adamantyl.

Ar¹ is preferably phenyl, isoquinolyl, piperidinyl, piperazinyl, morpholinyl, cyclohexyl, a six-membered heteroaromatic ring as defined above, such as pyridinyl, or adamantyl, unsubstituted or substituted with one two or three substituents as defined above. Thus Ar¹ may be phenyl, pyridinyl, piperidinyl, butyl, adamantyl or cyclohexyl. In particular, substituents are chosen from halogen, hydroxy, cyano, CF₃, SF₅, OCF₃, C₁₋₄alkyl, C₂₋₄alkenyl, C₂₋₄alkynyl, C₁₋₄alkoxy, C₁₋₄alkylthio, C₁₋₄alkylsulfinyl, C₁₋₄alkylsulfonyl, —NR⁶R⁷, cyanoC₁₋₄alkyl, haloC₁₋₄alkylcarbonyl, C₁₋₄alkylcarbonyl, CO₁₋₄alkoxycarbonyl, haloC₁₋₄alkyl, haloC₂₋₄alkenyl, hydroxyC₁₋₄alkyl, C₃₋₆cycloalkyl, cyanoC₃₋₆cycloalkyl, (halo)(hydroxy)C₁₋₄alkyl, C₁₋₄alkoxycarbonylC₁₋₄alkyl, phenyl, or a five-membered heteroaromatic ring as defined above where the phenyl or five-membered heteroaromatic ring is unsubstituted or substituted by C₁₋₄alkyl or halogen. More preferably the substituents are chosen from CF₃, OCF₃, SF₅, halogen, C₁₋₄alkyl, C₁₋₄alkoxy, —NR⁶R⁷, C₁₋₄alkylsulfonyl, cyanoC₁₋₄alkyl, cyanoC₁₋₄cycloalkyl, C₁₋₄alkylpyrazole, halophenyl, haloC₁₋₄alkylcarbonyl, phenyl, C₁₋₄alkoxycarbonylC₁₋₄alkyl, C₁₋₄cycloalkyl, (halo)(hydroxy)C₁₋₄alkyl, hydroxyC₁₋₄alkyl, haloC₁₋₄alkyl and C₁₋₄alkylcarbonyl. Thus the substituents can be chosen from CF₃, OCF₃, SF₅, methyl, tertiarybutyl, fluorine, chlorine, methoxy, isopropyl, methylthio, hydroxymethyl, methylsulfonyl, acetyl, 1-trifluoromethylethen-1-yl, 2-cyanoprop-2-yl, 1-cyanocycloprop-1-yl, bromine, 2-methylpyrazol-3-yl, 4-fluorophenyl, trifluoromethylcarbonyl, phenyl, 1-ethoxycarbonyl-1-methylethyl, cyclohexyl, 1-hydroxy-1-trifluoromethyl-2,2,2-trifluoroethyl, 1-hydroxy-2-methyl-2-propyl, cyano, ethoxycarbonyl, —OCH₂O—, —CH₂CH₂CH₂— and dimethylamino.

Ar¹ is preferably phenyl, naphthyl, quinolinyl, isoquinolinyl, or a six-membered heteroaromatic ring as defined as above, such as pyridyl, unsubstituted or substituted with one, two or three substituents as defined above. Thus Ar¹ may be phenyl, naphthyl, isoquinolinyl or pyridyl, particularly phenyl or pyridyl, especially phenyl. In particular Ar¹ may be unsubstituted or substituted with one or two substituents. Ar¹ may be unsubstituted. Ar¹ may be substituted. The substituents are preferably chosen from halogen, cyano, hydroxy, CF₃, OCF₃, C₁₋₄alkyl, C₂₋₄alkenyl, C₂₋₄alkynyl, C₁₋₄alkoxy, C₁₋₄alkylthio, —NR⁶R⁷, C₁₋₄alkoxycarbonyl and haloC₁₋₄alkyl. More preferably the substituents are chosen from CF₃, OCF₃, halogen, C₁₋₄alkyl, C₁₋₄alkoxy and —NR⁶R⁷. Thus the substituents can be chosen from CF₃, OCF₃, methyl, tertiarybutyl, fluorine, methoxy, isopropyl, methylthio, —OCH₂O—, CH₂CH₂CH₂—, cyano, chlorine and dimethylamine.

Thus preferred R¹ groups include 4-trifluoromethylphenyl, 4-tertiarybutylphenyl, phenyl, 2-trifluoromethylphenyl, 3-chlorophenyl, 3-trifluoromethylphenyl, 2,4-difluorophenyl, 4-methoxyphenyl, 2-isopropylphenyl, 3-methylthiophenyl, 2-naphthyl, 4-trifluoromethoxyphenyl, 1,3-benzodioxol-5-yl, 2-cyanophenyl, 2,4-dichlorophenyl, 3,4-dichlorophenyl, 4-dimethylaminophenyl, 2-methyl-4-chlorophenyl, 3-chloro-4-fluorophenyl, 2-fluoro-6-trifluoromethylphenyl 2-trifluoromethyl-4-fluorophenyl, 3-trifluoromethyl-4-fluorophenyl, 2-chloro-4-trifluoromethylphenyl, 2,3-dihydro-1H-inden-5-yl, 2,3-dihydro-1,4-benzodioxin-6-yl, 5-isoquinolyl, 2-trifluoromethylpyridin-6-yl and 3-trifluoromethylpyridin-6-yl.

Further preferred R¹ groups include 2-phenylethyl, 3-fluorophenylmethyl, diphenylmethyl, (1S)-1-phenylethyl and 3,4-dichlorophenylmethyl.

Yet further preferred R¹ groups include 4-fluorophenyl, 4-acetylphenyl, 4-methylthiophenyl, 1-trifluoromethylethen-1-ylphenyl, 4-(pentafluorothio)phenyl, 4-chlorophenyl, 4-methylphenyl, 4-hydroxymethylphenyl, 4-methylsulfonylphenyl, 2-chloropyrid-5-yl, 4-(1-cyano-1-methylethyl)phenyl, 4-(1-cyano-1-cyclopropyl)phenyl, 4-bromophenyl, 4-(2-methylpyrazol-3-yl)phenyl, 4-(4-fluorophenyl)phenyl, butyl adamant-1-yl, 1-trifluoroacetyl-4-piperidinyl, cyclohexyl, 1-phenylpiperidin-4-yl, 4-isopropylphenyl, 4-(1-ethoxycarbonyl-1-methylethyl)phenyl, 4-cyclohexylphenyl, 4-(1-hydroxy-1-trifluoromethyl-2,2,2-trifluoroethyl)phenyl, 4-(1-hydroxy-2-methyl-2-propyl)phenyl, 4-trifluoromethylphenylethyl, 4-cyanophenyl, 4-tert.butylcyclohexyl, 1-ethoxycarbonylpiperidin-4-yl, 3-methylpyridin-6-yl, 2-trifluoromethypyridin-4-yl, 2-fluoro-4-trifluoromethylphenyl, 4-trifluoromethoxyphenyl and 3-fluoro-4-trifluoromethylphenyl. R¹ can be 4-trifluoromethylphenyl.

Ar is preferably phenyl or a 5- or 6-membered ring containing one or two nitrogen atoms. Ar is more preferably phenyl, pyridyl or imidazolyl, especially pyridyl such as pyrid-2-yl such as 3-substituted pyrid-2-yl. Ar may also be pyridazinyl.

Ar is preferably unsubstituted or substituted with one or two substituents. More particularly Ar is substituted with one substituent, particularly ortho to the point of attachment to the rest of the molecule.

The substituents on Ar are preferably chosen from halogen, CF₃, OCF₃, C₁₋₄alkyl, C₁₋₄alkoxy, C₁₋₄alkylcarbonyl, cyano, hydroxyC₁₋₄alkyl and a five-membered heteroaromatic ring as defined above, such as thiazolyl or pyrazolyl, optionally substituted by C₁₋₄alkyl, such as methyl.

The substituents on Ar are more preferably chosen from halogen, CF₃, OCF₃, C₁₋₄alkyl, C₁₋₄alkoxy, —NR⁶R⁷, haloC₁₋₄alkyl and aminoC₁₋₄alkyl. More preferably they are chosen from halogen, CF₃, C₁₋₂alkoxy and C₁₋₂alkyl, such as CF₃, methyl and methoxy. Thus Ar can be 3-trifluoromethylpyrid-2-yl, 3 methylpyrid-2-yl, 3-methoxypyrid-2-yl, 4-trifluoromethylphenyl or 1-methylimidazol-2-yl. Ar can also be 3-chloropyrid-2-yl, 3-bromopyrid-2-yl, 3-(thiazol-2-yl)pyrid-2-yl, 3-(2-methylpyrazol-3-yl)pyrid-2-yl, 3-acetylpyrid-2-yl, 3-cyanopyrid-2-yl, 3-(2-hydroxyprop-2-yl)pyrid-2-yl, 4-methylpyridazin-3-yl, 4-trifluoromethylpyridazin-3-yl and 2-methoxyphenyl. Ar can be 3-trifluoromethylpyrid-2-yl.

R² is preferably hydrogen, halogen, CF₃, C₁₋₄alkyl, C₁₋₄alkoxy, OCF₃, —NR⁶R⁷, —CO₂H, cyano, amido, phenyl, pyridyl, morpholinyl, imidazolyl or C₁₋₄alkylimidazolyl. These groups may be joined to the rest of the molecule via an ethylene or methylene linker which, when present, is preferably methylene.

R² and R³ are thus preferably hydrogen, halogen, CF₃, C₁₋₂alkyl, C₁₋₂alkoxy, OCF₃ or —NR⁶R⁷. R² and R³ are particularly hydrogen or halogen such as chlorine. R² and R⁵ are generally hydrogen. Particular embodiments of R² are hydrogen, cyano, bromine, 1-methylimidazol-2-yl, methyl, amido, phenyl, pyrid-4-yl, pyrid-3-yl, morpholin-4-ylmethyl, dimethylaminomethyl, imidazol-1-ylmethyl and carboxyl. R³ may be hydrogen, halogen, such as bromine or chlorine, or cyano.

R⁶ and R⁷ are preferably hydrogen, methyl or ethyl. R⁶ and R⁷ can both be hydrogen, one can be hydrogen and the other can be methyl. In one embodiment they are both methyl.

n is generally 0, 1 or 2, preferably 0 or 1 and most often 0.

In one embodiment the compound of formula I is a free base. It can also be a hydrochloride salt.

The present invention also provides compounds of formula IA: 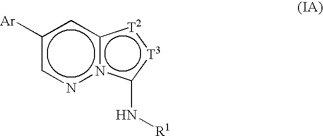
in which T², T³, Ar and R¹ are as defined above. The preferred definitions of these substituents apply to this subgenus.

Compounds of formula IA are preferred in which R² is hydrogen, Ar is phenyl or pyridyl which is unsubstituted or substituted by methyl, CF₃ or methoxy and R¹ is phenyl substituted generally at the 4-position by CF₃. More particularly Ar is pyridyl, such as pyrid-2-yl, substituted, preferably at the 3-position, by CF₃.

The present invention also provides compounds of formula IB: 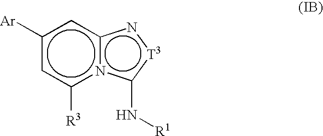
in which Ar, R¹, R³ and T³ are as defined above for formula I including the preferences listed. In one embodiment of the compounds of formula IB, T³ is N.

Compounds of formula IB are preferred in which Ar is pyridyl, particularly when substituted by hydroxy, methyl, methoxy or CF₃, R¹ is phenyl, particularly when substituted by CF₃, and R³ is hydrogen or chlorine. Ar may be substituted by methyl, methoxy or CF₃. Particular preference is for compounds where Ar is pyrid-2-yl substituted at the 3-position and R¹ is 4-trifluoromethylphenyl.

The present invention also provides compounds of formula IC: 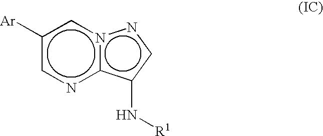
in which Ar and R¹ are as defined above for formula I including the preferences listed. Particularly preferred are compounds in which Ar is pyridyl, particularly when substituted by CF₃, and R¹ is phenyl, particularly when substituted by CF₃. Ar is generally pyrid-2-yl preferably substituted at the 3-position and R¹ is 4-trifluoromethylphenyl.

The present invention also provides compounds of formula ID: 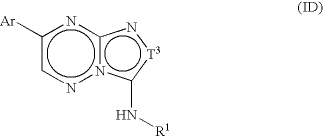
in which Ar, R¹ and T³ are as defined above for formula I including the preferences listed. In one embodiments T³ in the compounds of formula ID is N. Preferred are compounds in which Ar is pyridyl, particularly when substituted by CF₃ or Cl, and R¹ is phenyl, particularly when substituted by CF₃, cyano or chlorine. Particularly preferred are compounds in which Ar is pyridyl particularly when substituted by CF₃, and R¹ is phenyl, particularly when substituted by CF₃. Ar is generally pyrid-2-yl preferably substituted at the 3-position and R¹ is 4-trifluoromethylphenyl. R¹ may be 4-chlorophenyl or 4-cyanophenyl.

The present invention provides compounds of formula IE: 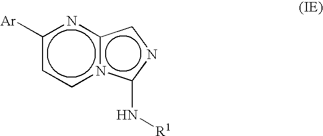
in which Ar and R¹ are as defined above for formula I including the preferences listed. Particularly preferred are compounds in which Ar is pyridyl, particularly when substituted by CF₃, and R¹ is phenyl, particularly when substituted by CF₃. Ar is generally pyrid-2-yl preferably substituted at the 3-position and R¹ is 4-trifluoromethylphenyl.

Particular embodiments of the invention include:
N-(4-trifluoromethylphenyl)-7-(3-trifluoromethyl-2-pyridyl)[1,2,4]triazolo[4,3-b]pyridazine-3-amine;
N-(4-tert-butyl)phenyl)-7-(3-trifluoromethyl-2-pyridyl)[1,2,4]triazolo[4,3-b]pyridazine-3-amine;
N-phenyl-7-[3-trifluoromethyl-2-pyridinyl][1,2,4]triazolo[4,3-b]pyridazin-3-amine;
N-[2-trifluoromethylphenyl]-7-[3-trifluoromethyl-2-pyridinyl][1,2,4]triazolo[4,3-b]pyridazin-3-amine;
N-(3-chlorophenyl)-7-[3-trifluoromethyl-2-pyridinyl][1,2,4]triazolo[4,3-b]pyridazin-3-amine;
N-[3-trifluoromethylphenyl]-7-[3-trifluoromethyl-2-pyridinyl][1,2,4]triazolo[4,3-b]pyridazin-3-amine;
N-(2,4-difluorophenyl)-7-[3-trifluoromethyl-2-pyridinyl][1,2,4]triazolo[4,3-b]pyridazin-3-amine;
N-[4-methoxyphenyl]-7-[3-trifluoromethyl-2-pyridinyl][1,2,4]triazolo[4,3-b]pyridazin-3-amine;
N-[2-(1-methylethyl)phenyl]-7-[3-trifluoromethyl-2-pyridinyl][1,2,4]triazolo[4,3-b]pyridazin-3-amine;
N-[3-methylsulfanylphenyl]-7-[3-trifluoromethyl-2-pyridinyl][1,2,4]triazolo[4,3-b]pyridazin-3-amine;
N-(2-naphthalenyl)-7-[3-trifluoromethyl-2-pyridinyl][1,2,4]triazolo[4,3-b]pyridazin-3-amine;
N-{4-trifluoromethoxyphenyl}-7-[3-trifluoromethyl-2-pyridinyl][1,2,4]triazolo[4,3-b]pyridazin-3-amine;
N-(2-phenylethyl)-7-[3-trifluoromethyl-pyridinyl][1,2,4]triazolo[4,3-b]pyridazin-3-amine;
N-(1,3-benzodioxol-5-yl)-7-[3-trifluoromethyl-2-pyridinyl][1,2,4]triazolo[4,3-b]pyridazin-3-amine;
N-[3-fluorophenylmethyl]-7-[3-trifluoromethyl-2-pyridinyl][1,2,4]triazolo[4,3-b]pyridazin-3-amine;
2-({7-[3-trifluoromethyl-2-pyridinyl][1,2,4]triazolo[4,3-b]pyridazin-3-yl}amino)benzonitrile;
N-(diphenylmethyl)-7-[3-trifluoromethyl-2-pyridinyl][1,2,4]triazolo[4,3-b]pyridazin-3-amine;
N-[(1S)-1-phenylethyl]-7-[3-trifluoromethyl-2-pyridinyl][1,2,4]triazolo[4,3-b]pyridazin-3-amine;
N-(2,4-dichlorophenyl)-7-[3-trifluoromethyl-2-pyridinyl][1,2,4]triazolo[4,3-b]pyridazin-3-amine;
N-(3,4-dichlorophenyl)-7-[3-trifluoromethyl-2-pyridinyl][1,2,4]triazolo[4,3-b]pyridazin-3-amine;
N-[4-dimethylaminophenyl]-N-{7-[3-trifluoromethyl-2-pyridinyl][1,2,4]triazolo[4,3-b]pyridazin-3-yl}amine;
N-[(3,4-dichlorophenyl)methyl]-7-[3-trifluoromethyl-2-pyridinyl][1,2,4]triazolo[4,3-b]pyridazin-3-amine;
N-(4-chloro-2-methylphenyl)-7-[3-trifluoromethyl-2-pyridinyl][1,2,4]triazolo[4,3-b]pyridazin-3-amine;
N-(3-chloro-4-fluorophenyl)-7-[3-trifluoromethyl-2-pyridinyl][1,2,4]triazolo[4,3-b]pyridazin-3-amine;
N-[2-fluoro-6-trifluoromethylphenyl]-7-[3-trifluoromethyl-2-pyridinyl][1,2,4]triazolo[4,3-b]pyridazin-3-amine;
N-[4-fluoro-2-trifluoromethylphenyl]-7-[3-trifluoromethyl-2-pyridinyl][1,2,4]triazolo[4,3-b]pyridazin-3-amine;
N-[4-fluoro-3-trifluoromethylphenyl]-7-[3-trifluoromethyl-2-pyridinyl][1,2,4]triazolo[4,3-b]pyridazin-3-amine;
N-[2-chloro-4-trifluoromethylphenyl]-7-[3-trifluoromethyl-2-pyridinyl][1,2,4]triazolo[4,3-b]pyridazin-3-amine;
N-(2,3-dihydro-1H-inden-5-yl)-7-[3-trifluoromethyl-2-pyridinyl][1,2,4]triazolo[4,3-b]pyridazin-3-amine;
N-(2,3-dihydro-1,4-benzodioxin-6-yl)-7-[3-trifluoromethyl-2-pyridinyl][1,2,4]triazolo[4,3-b]pyridazin-3-amine;
N-(4-trifluoromethylphenyl)-7-(3-methyl-2-pyridyl)[1,2,4]triazolo[4,3-b]pyridazine-3-amine;
5-chloro-7-(3-methyl-2-pyridyl)-N-(4-trifluoromethylphenyl)[1,2,4]triazolo[4,3-a]pyridin-3-amine;
5-chloro-7-(2-methoxyphenyl)-N-(4-trifluoromethylphenyl)[1,2,4]triazolo[4,3-a]pyridin-3-amine;
6-chloro-N-(4-trifluoromethylphenyl)-7-(3-trifluoromethyl-2-pyridyl)[1,2,4]triazolo[4,3-a]pyridin-3-amine;
6-chloro-N-(5-isoquinolyl)-7-(4-trifluoromethylphenyl)[1,2,4]triazolo[4,3-b]pyridazin-3-amine;
7-(3-methyl-2-pyridyl)-N-(4-trifluoromethylphenyl)[1,2,4]triazolo[4,3-a]pyridin-3-amine;
7-(3-trifluoromethyl-2-pyridyl)-N-(4-trifluoromethylphenyl)[1,2,4]triazolo[4,3-a]pyridin-3-amine;
7-(2-methoxyphenyl)-N-(4-trifluoromethylphenyl)[1,2,4]triazolo[4,3-a]pyridin-3-amine;
N-(5-isoquinolyl)-7-(4-trifluoromethylphenyl)[1,2,4]triazolo[4,3-b]pyridazine-3-amine;
7-(3-trifluoromethyl-2-pyridyl)-N-(5-trifluoromethyl-2-pyridyl)[1,2,4]triazolo[4,3-b]pyridazine-3-amine;
N-(4-trifluoromethylphenyl)-6-(3-trifluoromethylpyrid-2-yl)pyrazolo[1,5-a]pyrimidin-3-amine;
4-trifluoromethylphenyl-3-(3-trifluoromethylpyridin-2-yl)imidazo[1,5-b]pyridazin-7-ylamine;
N-[4-trifluoromethylphenyl]-7-[3-trifluoromethylpyridin-2-yl]imidazo[1,2-b]pyridazin-3-amine;
7-[3-trifluoromethylpyridin-2-yl]-N-[5-trifluoromethylpyridin-2-yl]imidazo[1,2-b]pyridazin-3-amine;
N-(5-methylpyridin-2-yl)-7-[3-trifluoromethylpyridin-2-yl]imidazo[1,2-b]pyridazin-3-amine;
[7-(3-methylpyridin-2-yl)-[1,2,4]triazolo[4,3-b][1,2,4]triazin-3-yl]-(4-trifluoromethyl-phenyl)amine; and
[7-(1-methyl-1H-imidazol-2-yl)[1,2,4]triazolo[4,3-b]pyridazin-3-yl]-(4-trifluoromethylphenyl)amine;
   or a pharmaceutically acceptable salt thereof.

Further preferred compounds include:
7-(3-chloro-2-pyridyl)-N-(4-trifluoromethylphenyl)[1,2,4]triazolo[4,3-b]pyridazine-3-amine;
7-(3-bromo-2-pyridyl)-N-(4-trifluoromethylphenyl)[1,2,4]triazolo[4,3-b]pyridazine-3-amine;
7-[3-(1,3-thiazol-2-yl)pyridin-2-yl]-N-(4-trifluoromethylphenyl)[1,2,4]triazolo[4,3-b]pyridazine-3-amine;
7-[3-(1-methyl-1H-pyrazol-5-yl)pyridin-2-yl)-N-(4-trifluoromethylphenyl) [1,2,4]triazolo[4,3-b]pyridazine-3-amine;
7-(3-ethoxycarbonyl-2-pyridyl)-N-(4-trifluoromethylphenyl)[1,2,4]triazolo[4,3-b]pyridazine-3-amine;
7-(3-cyano-2-pyridyl)-N-4-trifluoromethylphenyl[1,2,4]triazolo[4,3-b]pyridazine-3-amine;
N-(4-chlorophenyl)-7-(3-trifluoromethyl-2-pyridyl)[1,2,4]triazolo[4,3-b]pyridazine-3-amine;
N-(4-tolyl)-7-(3-trifluoromethyl-2-pyridyl) [1,2,4]triazolo[4,3-b]pyridazine-3-amine;
N-(4-(2-hydroxyethyl)phenyl)-7-(3-trifluoromethyl-2-pyridyl)[1,2,4]triazolo[4,3-b]pyridazine-3-amine;
N-(4-methylsulfonylphenyl)-7-(3-trifluoromethyl-2-pyridyl)[1,2,4]triazolo[4,3-b]pyridazine-3-amine;
N-(2-chloro-5-pyridyl)-7-(3-trifluoromethyl-2-pyridyl)[1,2,4]triazolo[4,3-b]pyridazine-3-amine;
N-(4-(1-cyano-1-methylethyl)phenyl)-7-(3-trifluoromethyl-2-pyridyl)[1,2,4]triazolo[4,3-b]pyridazine-3-amine;
N-(4-(1-cyclopropylphenyl)-7-(3-trifluoromethyl-2-pyridyl) [1,2,4]triazolo[4,3-b]pyridazine-3-amine;
N-(4-bromophenyl)-7-(3-trifluoromethyl-2-pyridyl)[1,2,4]triazolo[4,3-b]pyridazine-3-amine;
N-(4-(2-methyl-3-pyrazolo)phenyl)-7-(3-trifluoromethyl-2-pyridyl)[1,2,4]triazolo[4,3-b]pyridazine-3-amine;
N-(4-(4-fluorophenyl)phenyl)-7-(3-trifluoromethyl-2-pyridyl)[1,2,4]triazolo[4,3-b]pyridazine-3-amine;
N-butyl-7-(3-trifluoromethyl-2-pyridyl)[1,2,4]triazolo[4,3-b]pyridazine-3-amine;
N-(1-adamantyl)-7-(3-trifluoromethyl-2-pyridyl)[1,2,4]triazolo[4,3-b]pyridazine-3-amine;
N-(1-trifluoroacetyl-4-piperidinyl)-7-(3-trifluoromethyl-2-pyridyl)[1,2,4]triazolo[4,3-b]pyridazine-3-amine;
N-(1-cyclohexyl)-7-(3-trifluoromethyl-2-pyridyl)[1,2,4]triazolo[4,3-b]pyridazine-3-amine;
N-(1-phenyl-4-piperidinyl)-7-(3-trifluoromethyl-2-pyridyl)[1,2,4]triazolo[4,3-b]pyridazine-3-amine;
N-(4-trifluoromethylphenyl)-7-(2-cyanophenyl)-[1,2,4]triazolo[4,3-b]pyridazine-3-amine;
N-(4-trifluoromethylphenyl)-7-(3-(1-hydroxy-1-methylethyl)-2-pyridyl-[1,2,4]triazolo[4,3-b]pyridazine-3-amine;
N-(4-(1-methylethylphenyl)-7-(3-trifluoromethyl-2-pyridyl)[1,2,4]triazolo[4,3-b]pyridazine-3-amine;
N-(4-(1-ethoxycarbonyl-1-methylethyl)phenyl)-7-(3-trifluoromethyl-2-pyridyl)[1,2,4]triazolo[4,3-b]pyridazine-3-amine;
N-(4-cyclohexylphenyl)-7-(3-trifluoromethyl-2-pyridyl)[1,2,4]triazolo[4,3-b]pyridazine-3-amine;
N-(4-(1-hydroxy-1-trifluoromethyl-2,2,2-trifluoroethyl)phenyl)-7-(3-trifluoromethyl-2-pyridyl)[1,2,4]triazolo[4,3-b]pyridazine-3-amine;
N-(4-(1-hydroxy-2-methyl-2-propyl)phenyl)-7-(3-trifluoromethyl-2-pyridyl) [1,2,4]triazolo[4,3-b]pyridazine-3-amine;
N-(2-4-trifluoromethylphenylethyl-7-(3-trifluoromethyl-2-pyridyl)[1,2,4]triazolo[4,3-b]pyridazine-3-amine;
N-(trans)-(4-tert.-butylcyclohexyl)-7-(3-trifluoromethyl-2-pyridyl)[1,2,4]triazolo[4,3-b]pyridazine-3-amine;
N-(1-ethoxycarbonyl-4-piperidinyl)-7-(3-trifluoromethyl-2-pyridyl)[1,2,4]triazolo[4,3-b]pyridazine-3-amine;
7-(4-methylpyridazin-3-yl)-N-[4-trifluoromethylphenyl][1,2,4]triazolo[4,3-b]pyridazin-3-amine;
N-[4-trifluoromethylphenyl]-7-[5-trifluoromethylpyrimidin-4-yl][1,2,4]triazolo[4,3-b]pyridazin-3-amine;
5-bromo-N-[4-trifluoromethylphenyl]-3-[3-trifluoromethylpyridin-2-yl]imidazo [1,5-b]pyridazin-7-amine;
5-(1-methyl-1H-imidazol-2-yl)-N-[4-trifluoromethylphenyl]-3-[3-trifluoromethyl pyridin-2-yl]imidazo[1,5-b]pyridazin-7-amine;
N-(4-chlorophenyl)-3-[3-trifluoromethylpyridin-2-yl]imidazo[1,5-b]pyridazin-7-amine;
5-methyl-N-[4-trifluoromethylphenyl]-3-[3-trifluoromethylpyridin-2-yl]imidazo[1,5-b]pyridazin-7-amine;
7-{[4-trifluoromethylphenyl]amino}-3-[3-trifluoromethylpyridin-2-yl]imidazo[1,5-b]pyridazine-5-carbonitrile;
7-{[4-trifluoromethylphenyl]amino}-3-[3-trifluoromethylpyridin-2-yl]imidazo[1,5-b]pyridazine-5-carboxamide;
3-(3-methylpyridin-2-yl)-N-[4-trifluoromethylphenyl]imidazo[1,5-b]pyridazin-7-amine;
3-(3-methylpyridin-2-yl)-7-{[4-trifluoromethylphenyl]amino}imidazo[1,5-b]pyridazine-5-carbonitrile;
5-phenyl-N-[4-trifluoromethylphenyl]-3-[3-trifluoromethylpyridin-2-yl]imidazo [1,5-b]pyridazin-7-amine;
5-pyridin-4-yl-N-[4-trifluoromethylphenyl]-3-[3-trifluoromethylpyridin-2-yl]imidazo[1,5-b]pyridazin-7-amine;
5-pyridin-3-yl-N-[4-trifluoromethylphenyl]-3-[3-trifluoromethylpyridin-2-yl]imidazo[1,5-b]pyridazin-7-amine
5-(morpholin-4-ylmethyl)-N-[4-trifluoromethylphenyl]-3-[3-trifluoromethyl pyridin-2-yl]imidazo[1,5-b]pyridazin-7-amine;
5-[dimethylaminomethyl]-N-[4-trifluoromethylphenyl]-3-[3-trifluoromethyl pyridin-2-yl]imidazo[1,5-b]pyridazin-7-amine;
5-(1H-imidazol-1-ylmethyl)-N-[4-trifluoromethylphenyl]-3-[3-trifluoromethyl pyridin-2-yl]imidazo[1,5-b]pyridazin-7-amine;
7-{[4-trifluoromethylphenyl]amino}-3-[3-trifluoromethylpyridin-2-yl]imidazo[1,5-b]pyridazine-5-carboxylic acid;
7-[1-oxido-3-trifluoromethylpyridin-2-yl]-N-[4-trifluoromethylphenyl]-imidazo[1,2-b]pyridazin-3-amine;
2-bromo-N-[4-trifluoromethylphenyl]-7-[3-trifluoromethylpyridin-2-yl]imidazo[1,2-b]pyridazin-3-amine;
3-{[4-trifluoromethylphenyl]amino}-7-[3-trifluoromethylpyridin-2-yl]imidazo[1,2-b]pyridazine-2-carbonitrile;
2-methyl-N-[4-trifluoromethylphenyl]-7-[3-trifluoromethylpyridin-2-yl]imidazo [1,2-b]pyridazin-3-amine;
7-[3-trifluoromethylpyridin-2-yl]-N-[6-trifluoromethylpyridin-3-yl]imidazo[1,2-b]pyridazin-3-amine;
N-(4-chlorophenyl)-7-[3-trifluoromethylpyridin-2-yl]imidazo[1,2-b]pyridazin-3-amine;
N-[2-fluoro-4-trifluoromethylphenyl]-7-[3-trifluoromethylpyridin-2-yl]imidazo[1,2-b]pyridazin-3-amine;
N-(6-methylpyridin-3-yl)-7-[3-trifluoromethylpyridin-2-yl]imidazo[1,2-b]pyridazin-3-amine;
N-[4-trifluoromethoxyphenyl]-7-[3-trifluoromethylpyridin-2-yl]imidazo[1,2-b]pyridazin-3-amine;
N-[3-fluoro-4-trifluoromethylphenyl]-7-[3-trifluoromethylpyridin-2-yl]imidazo [1,2-b]pyridazin-3-amine;
7-(3-chloropyridin-2-yl)-N-[4-trifluoromethylphenyl]imidazo[1,2-b]pyridazin-3-amine;
N-(4-chlorophenyl)-7-(3-chloropyridin-2-yl)imidazo[1,2-b]pyridazin-3-amine;
[7-(3-trifluoromethyl-pyridin-2-yl)-[1,2,4]triazolo[4,3-b][1,2,4]triazin-3-yl]-(4-trifluoromethyl-phenyl)-amine;
N-(4-chlorophenyl)-7-[3-trifluoromethylpyridin-2-yl][1,2,4]triazolo[4,3-b][1,2,4]triazin-3-amine;
4-({7-[3-trifluoromethylpyridin-2-yl][1,2,4]triazolo[4,3-b][1,2,4]triazin-3-yl}amino)benzonitrile;
7-(3-chloropyridin-2-yl)-N-[4-trifluoromethylphenyl][1,2,4]triazolo[4,3-b][1,2,4]triazin-3-amine;
N-(4-chlorophenyl)-7-(3-chloropyridin-2-yl)[1,2,4]triazolo[4,3-b][1,2,4]triazin-3-amine;
3-(3-methylpyridin-2-yl)-N-[4-trifluoromethylphenyl]imidazo[1,2-b][1,2,4]triazin-7-amine;
3-(3-chloropyridin-2-yl)-N-[4-trifluoromethylphenyl]imidazo[1,2-b][1,2,4]triazin-7-amine;
N-[4-trifluoromethylphenyl]-3-[3-trifluoromethylpyridin-2-yl]imidazo[1,2-b][1,2,4]triazin-7-amine;
N-[4-trifluoromethylphenyl]-7-[3-trifluoromethylpyridin-2-yl]imidazo[1,2a]pyridin-3-amine;
N-[4-trifluoromethylphenyl]-2-[3-trifluoromethylpyridin-2-yl]imidazo[1,5-a]pyrimidin-6-amine;
N-(4-trifluoromethylphenyl)-7-(2-methoxyphenyl)[1,2,4]triazolo[4,3-b]pyridazine-3-amine;
   or a pharmaceutically acceptable salt thereof.

Further compounds of the invention include:
N-(4-fluorophenyl)-7-(3-trifluoromethylpyrid-2-yl)[1,2,4]triazolo[4,3-b]pyridazine-3-amine;
N-(4-acetylphenyl-7-(3-trifluoromethylpyrid-2-yl)[1,2,4]txiazolo[4,3-b]pyridazine-3-amine;
N-(3-trifluoromethylpyrid-4-yl)-7-(3-trifluoromethylpyrid-2-yl)[1,2,4]triazolo[4,3-b]pyridazine-3-amine;
N-(4-methylthiophenyl)-7-(3-trifluoromethylpyrid-2-yl)[1,2,4]triazolo[4,3-b]pyridazine-3-amine;
N-(1-trifluoromethylethen-1-yl)-7-(3-trifluoromethylpyrid-2-yl)[1,2,4]triazolo[4,3-b]pyridazine-3-amine;
N-(3-trifluoromethylpyrid-6-yl)-7-(3-trifluoromethylpyrid-2-yl)[1,2,4]triazolo[4,3-a]pyridine-3-amine;
N-(4-pentafluorothiophenyl)-7-(3-trifluoromethylpyrid-2-yl)[1,2,4]triazolo[4,3-b]pyridazine-3-amine; and
N-(4-cyanophenyl)-7-(3-trifluoromethylpyrid-2-yl)[1,2,4]triazolo[4,3-b]pyridazine-3-amine;
   or a pharmaceutically acceptable salt thereof.

As used herein, the term “alkyl” or “alkoxy” as a group or part of a group means that the group is straight or branched. Examples of suitable alkyl groups include methyl, ethyl, n-propyl, i-propyl, n-butyl, s-butyl and t-butyl. Examples of suitable alkoxy groups include methoxy, ethoxy, n-propoxy, i-propoxy, n-butoxy, s-butoxy and t-butoxy. “Alkylthio”, “alkylsulfinyl” and “alkylsulfonyl” shall be construed in an analogous manner.

As used herein, the term “hydroxyC₁₋₆alkyl” means a C₁₋₆alkyl group in which one or more (in particular 1 to 3, and especially 1) hydrogen atoms have been replaced by hydroxy groups. Particularly preferred are hydroxyC₁₋₃alkyl groups, for example, CH₂OH, CH₂CH₂OH, CH(CH₃)OH or C(CH₃)₂OH, and most especially CH₂OH. “Aminoalkyl”, “cyanoalkyl” and “(halo)(hydroxy)alkyl” shall be construed in an analogous manner.

As used herein, the terms “haloC₁₋₆alkyl” and “haloC₁₋₆alkoxy” means a C₁₋₆alkyl or C₁₋₆alkoxy group in which one or more (in particular, 1 to 3) hydrogen atoms have been replaced by halogen atoms, especially fluorine or chlorine atoms. Preferred are fluoroC₁₋₆alkyl and fluoroC₁₋₆alkoxy groups, in particular, fluoroC₁₋₃alkyl and fluoroC₁₋₃alkoxy groups, for example, CF₃, CH₂CH₂F, CH₂CHF₂, CH₂CF₃, OCF₃, OCH₂CH₂F, OCH₂CF₂ or OCH₂CF₃, and most especially CF₃, OCF₃ and OCH₂CF₃.

As used herein, the terms “alkenyl” and “alkynyl” as a group or part of a group means that the group is straight or branched. Examples of suitable alkenyl groups include vinyl and alkyl. A suitable alkynyl group is acetylene or propargyl.

As used herein, the term “cycloalkyl” as a group or part of a group means that the group contains a cyclic portion. Examples of suitable cycloalkyl groups include cyclopropyl, methylcyclopropyl, cyclobutyl, cyclopentyl and cyclohexyl. Cyclohexyl groups, when substituted, may have a cis or trans configuration. Terms such as “halocycloalkyl”, “cyanocycloalkyl”, “hydroxycycloalkyl”, “aminocycloalkyl” and “(halo)(hydroxy)cycloalkyl” shall be construed analogously to the above definitions for alkyl derivatives.

When used herein, the term “halogen” means fluorine, chlorine, bromine and iodine. The most preferred halogens are fluorine and chlorine.

When used herein, the term “carboxy” as a group or part of a group denotes CO₂H.

When used herein, the term “C₁₋₆alkoxycarbonyl” denotes a C₁₋₆alkoxy or a haloC₁₋₆alkoxy radical attached via the oxygen atom thereof to a carbonyl (C═O) radical thus forming a C₁₋₆alkoxycarbonyl or haloC₁₋₆alkoxycarbonyl radical. Suitable examples of such esterified carboxy groups include, for example, methoxycarbonyl, ethoxycarbonyl, propoxycarbonyl, isopropoxycarbonyl and tertbutoxycarbonyl.

Examples of 6-membered heterocycles are pyridine, pyrimidine, pyrazine, pyridazine and triazine.

Examples of 5-membered heterocycles are thiophene, furan, pyrrole, imidazole, pyrazole, oxazole, isoxazole, thiazole, isothiazole, 1,2,3-triazole, 1,2,4-triazole, oxadiazole, thiadiazole and tetrazole.

As used herein, the term “fused 9 or 10 membered bicyclic heteroaromatic ring system” means a 5,6-, 6,5- or 6,6-fused ring system wherein one or both rings contain ring heteroatoms. The ring system is preferably aromatic or partially saturated, thus the ring system preferably comprises an aromatic 6-membered ring fused to a 5- or 6-membered ring which may be unsaturated, partially saturated or saturated. When the ring system contains more than one ring heteroatom at least one such heteroatom is nitrogen. It will be appreciated that where one of the ring heteroatoms is a nitrogen atom, such heteroatom may be at the bridgehead position of the fused ring system. It will also be appreciated that where one of the ring heteroatoms in a saturated ring is sulfur, such heteroatom may be oxidized to a S(O) or S(O)₂ moiety. Likewise, any carbon atom in a saturated ring may be oxidized to a C═O moiety.

Suitable examples of a “fused 9 or 10 membered heterobicyclic ring system” include isoquinolinyl, quinolinyl, cinnolinyl, phthalazinyl, quinazolinyl, quinoxalinyl, naphthyridinyl, indolyl, isoindolyl, benzofuranyl, benzothiophenyl, benzimidazolyl, indazolyl, benzoxazolyl, benzisoxazolyl, benzothiazolyl, benzisothiazolyl, benzotriazole, pyridopyridazinyl, pyridopyrimidinyl, pyridopyrazinyl, pyrrolopyridinyl, furopyridinyl, thienopyridinyl, pyrrolopyridazinyl, furopyridazinyl, thienopyridazinyl, pyridolopyrimidinyl, furopyrimidinyl, thienopyrimidinyl, pyrrolopyrazinyl, furopyrazinyl, thienopyrazinyl, imidazopyridinyl, pyrazolopyridinyl, oxazolopyridinyl, isoxazolopyridinyl, thiazolopyridinyl, isothiazolopyridinyl, imidazopyridazinyl, pyrazolopyridazinyl, oxazolopyridazinyl, isoxazolopyridazinyl, thiazolopyridazinyl, isothiazolopyridazinyl, imidazopyrimidinyl, pyrazolopyrimidinyl, oxazolopyrimidinyl, isoxazolopyrimidinyl, thiazolopyrimidinyl, isothiazolopyrimidinyl, imidazopyrazinyl, pyrazolopyrazinyl, oxazolopyrazinyl, isoxazolopyrazinyl, thiazolopyrazinyl, isothiazolopyrazinyl, triazolopyridinyl, benzotriazolyl, quinolinonyl, isoquinolinonyl, dihydroquinolinonyl, dihydroisoquinolinonyl, tetrahydroquinolinyl tetrahydroisoquinolinyl, dihydroquinazolinonyl, dihydrobenzoxainonyl, dihydrobenzothiadiazine oxide and dihydrobenzothiadiazine dioxide.

In a further aspect of the present invention, the compounds of formula (I) may be prepared in the form of a pharmaceutically acceptable salt, especially an acid addition salt.

For use in medicine, the salts of the compounds of formula (I) will be non-toxic pharmaceutically acceptable salts. Other salts may, however, be useful in the preparation of the compounds according to the invention or of their non-toxic pharmaceutically acceptable salts. Suitable pharmaceutically acceptable salts of the compounds of this invention include acid addition salts which may, for example, be formed by mixing a solution of the compound according to the invention with a solution of a pharmaceutically acceptable acid such as hydrochloric acid, fumaric acid, p-toluenesulphonic acid, maleic acid, succinic acid, acetic acid, citric acid, tartaric acid, carbonic acid, phosphoric acid or sulphuric acid. Salts of amine groups may also comprise quaternary ammonium salts in which the amino nitrogen atom carries a suitable organic group such as an alkyl, alkenyl, alkynyl or aralkyl moiety. Furthermore, where the compounds of the invention carry an acidic moiety, suitable pharmaceutically acceptable salts thereof may include metal salts such as alkali metal salts, e.g. sodium or potassium salts; and alkaline earth metal salts, e.g. calcium or magnesium salts.

The salts may be formed by conventional means, such as by reacting the free base form of the compound of formula (I) with one or more equivalents of the appropriate acid in a solvent or medium in which the salt is insoluble, or in a solvent such as water which is removed in vacuo or by freeze drying or by exchanging the anions of an existing salt for another anion on a suitable ion exchange resin.

The present invention also includes within its scope N-oxides of the compounds of formula (I) above. In general, such N-oxides may be formed on any available nitrogen atom. The N-oxides may be formed by conventional means, such as reacting the compound of formula (I) with oxone in the presence of wet alumina.

The present invention includes within its scope prodrugs of the compounds of formula (I) above. In general, such prodrugs will be functional derivatives of the compounds of formula (I) which are readily convertible in vivo into the required compound of formula (I). Conventional procedures for the selection and preparation of suitable prodrug derivatives are described, for example, in “Design of Prodrugs”, ed. H. Bundgaard, Elsevier, 1985.

A prodrug may be a pharmacologically inactive derivative of a biologically active substance (the “parent drug” or “parent molecule”) that requires transformation within the body in order to release the active drug, and that has improved delivery properties over the parent drug molecule. The transformation in vivo may be, for example, as the result of some metabolic process, such as chemical or enzymatic hydrolysis of a carboxylic, phosphoric or sulphate ester, or reduction or oxidation of a susceptible functionality.

The present invention includes within its scope solvates of the compounds of formula (I) and salts thereof, for example, hydrates.

The compounds according to the invention may have one or more asymmetric centres, and may accordingly exist both as enantiomers and as diastereoisomers. It is to be understood that all such isomers and mixtures thereof are encompassed within the scope of the present invention. Furthermore, the compounds of formula (I) may also exist in tautomeric forms and the invention includes within its scope both mixtures and separate individual tautomers.

The present invention further provides pharmaceutical compositions comprising one or more compounds of formula (I) in association with a pharmaceutically acceptable carrier or excipient.

Preferably the compositions according to the invention are in unit dosage forms such as tablets, pills, capsules, powders, granules, sterile parenteral solutions or suspensions, metered aerosol or liquid sprays, drops, ampoules, auto-injector devices, suppositories, creams or gels; for oral, parenteral, intrathecal, intranasal, sublingual, rectal or topical administration, or for administration by inhalation or insufflation. Oral compositions such as tablets, pills, capsules or wafers are particularly preferred. For preparing solid compositions such as tablets, the principal active ingredient is mixed with a pharmaceutical carrier, e.g. conventional tabletting ingredients such as corn starch, lactose, sucrose, sorbitol, talc, stearic acid, magnesium stearate, dicalcium phosphate or gums, and other pharmaceutical diluents, e.g. water, to form a solid pre-formulation composition containing a homogeneous mixture of a compound of the present invention, or a pharmaceutically acceptable salt thereof. When referring to these pre-formulation compositions as homogeneous, it is meant that the active ingredient is dispersed evenly throughout the composition so that the composition may be readily subdivided into equally effective unit dosage forms such as tablets, pills and capsules. This solid pre-formulation composition is then subdivided into unit dosage forms of the type described above containing from 0.1 to about 500 mg of the active ingredient of the present invention. Favoured unit dosage forms contain from 1 to 500 mg, for example 1, 5, 10, 25, 60, 100, 300 or 500 mg, of the active ingredient. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The liquid forms in which the novel compositions of the present invention may be incorporated for administration orally or by injection include aqueous solutions, suitably flavoured syrups, aqueous or oil suspensions, and flavoured emulsions with edible oils such as cottonseed oil, sesame oil, coconut oil or peanut oil, as well as elixirs and similar pharmaceutical vehicles. Suitable dispersing or suspending agents for aqueous suspensions include synthetic and natural gums such as tragacanth, acacia, alginate, dextran, sodium carboxymethylcellulose, methylcellulose, polyvinyl-pyrrolidone or gelatin.

In the treatment of painful conditions such as those listed below, a suitable dosage level is about 1.0 mg to 15 g per day, preferably about 5.0 mg to 1 g per day, more preferably about 5 mg to 500 mg per day, especially 10 mg to 100 mg per day. The compounds may be administered on a regimen of 1 to 4 times per day.

It will be appreciated that the amount of a compound of formula (I) required for use in any treatment will vary not only with the particular compounds or composition selected but also with the route of administration, the nature of the condition being treated, and the age and condition of the patient, and will ultimately be at the discretion of the attendant physician.

The invention further provides a compound of formula (I) as defined above, or a pharmaceutically acceptable salt thereof, for use in treatment of the human or animal body. Preferably, said treatment is for a condition which is susceptible to treatment by modulation (preferably antagonism) of VR1 receptors.

The compounds of the present invention will be of use in the prevention or treatment of diseases and conditions in which pain and/or inflammation predominates, including chronic and acute pain conditions. Such conditions include rheumatoid arthritis; osteoarthritis; post-surgical pain; musculo-skeletal pain, particularly after trauma; spinal pain; myofascial pain syndromes; headache, including migraine, acute or chronic tension headache, cluster headache, temporomandibular pain, and maxillary sinus pain; ear pain; episiotomy pain; burns, and especially primary hyperalgesia associated therewith; deep and visceral pain, such as heart pain, muscle pain, eye pain, orofacial pain, for example, odontalgia, abdominal pain, gynaecological pain, for example, dysmenorrhoea, pain associated with cystitis and labour pain, chronic pelvic pain, chronic prostatitis and endometriosis; pain associated with nerve and root damage, such as pain associated with peripheral nerve disorders, for example, nerve entrapment and brachial plexus avulsions, amputation, peripheral neuropathies, tic douloureux, atypical facial pain, nerve root damage, and arachnoiditis; itching conditions including pruritis, itch due to hemodialysis, and contact dermatitis; pain (as well as broncho-constriction and inflammation) due to exposure (e.g. via ingestion, inhalation, or eye contact) of mucous membranes to capsaicin and related irritants such as tear gas, hot peppers or pepper spray; neuropathic pain conditions such as diabetic neuropathy, chemotherapy-induced neuropathy and post-herpetic neuralgia; “non-painful” neuropathies; complex regional pain syndromes; pain associated with carcinoma, often referred to as cancer pain; central nervous system pain, such as pain due to spinal cord or brain stem damage, low back pain, sciatica and ankylosing spondylitis; gout; scar pain; irritable bowel syndrome; inflammatory bowel disease; urinary incontinence including bladder detrusor hyper-reflexia and bladder hypersensitivity; respiratory diseases including chronic obstructive pulmonary disease (COPD), chronic bronchitis, cystic fibrosis, asthma and rhinitis, including allergic rhinitis such as seasonal and perennial rhinitis, and non-allergic rhinitis; autoimmune diseases; and immunodeficiency disorders.

Thus, according to a further aspect, the present invention provides a compound of formula (I) for use in the manufacture of a medicament for the treatment or prevention of physiological disorders that may be ameliorated by modulating VR1 activity.

The present invention also provides a method for the treatment or prevention of physiological disorders that may be ameliorated by modulating VR1 activity, which method comprises administration to a patient in need thereof of an effective amount of a compound of formula (I) or a composition comprising a compound of formula (I).

According to a further or alternative aspect, the present invention provides a compound of formula (I) for use in the manufacture of a medicament for the treatment or prevention of a disease or condition in which pain and/or inflammation predominates.

The present invention also provides a method for the treatment or prevention of a disease or condition in which pain and/or inflammation predominates, which method comprises administration to a patient in need thereof of an effective amount of a compound of formula (I) or a composition comprising a compound of formula (I).

According to a further aspect of the present invention, it may be desirable to treat any of the aforementioned conditions with a combination of a compound according to the present invention and one or more other pharmacologically active agents suitable for the treatment of the specific condition. The compound of formula (I) and the other pharmacologically active agent(s) may be administered to a patient simultaneously, sequentially or in combination. Thus, for example, for the treatment or prevention of pain and/or inflammation, a compound of the present invention may be used in conjunction with other analgesics, such as acetaminophen (paracetamol), aspirin and other NSAIDs, including selective cyclooxygenase-2 (COX-2) inhibitors, as well as opioid analgesics, especially morphine, NR2B antagonists, bradykinin antagonists, anti-migraine agents, anticonvulsants such as oxcarbazepine and carbamazepine, antidepressants (such as TCAs, SSRRIs, SNRIs, substance P antagonists, etc.), spinal blocks, gabapentin, pregabalin and asthma treatments (such as [symbol] ₂-adrenergic receptor agonists or leukotriene D₄antagonists (e.g. montelukast).

Specific anti-inflammatory agents include diclofenac, ibuprofen, indomethacin, nabumetone, ketoprofen, naproxen, piroxicam and sulindac, etodolac, meloxicam, rofecoxib, celecoxib, etoricoxib, parecoxib, valdecoxib and tilicoxib. Suitable opioid analgesics of use in conjunction with a compound of the present invention include morphine, codeine, dihydrocodeine, diacetylmorphine, hydrocodone, hydromorphone, levorphanol, oxymorphone, alfentanil, buprenorphine, butorphanol, fentanyl, sufentanyl, meperidine, methadone, nalbuphine, propoxyphene and pentazocine; or a pharmaceutically acceptable salt thereof. Suitable anti-migraine agents of use in conjunction with a compound of the present invention include CGRP-antagonists, ergotamines or 5-HT₁ agonists, especially sumatriptan, naratriptan, zolmatriptan or rizatriptan.

Therefore, in a further aspect of the present invention, there is provided a pharmaceutical composition comprising a compound of the present invention and an analgesic, together with at least one pharmaceutically acceptable carrier or excipient.

In a further or alternative aspect of the present invention, there is provided a product comprising a compound of the present invention and an analgesic as a combined preparation for simultaneous, separate or sequential use in the treatment or prevention of a disease or condition in which pain and/or inflammation predominates.

Compounds of formula I in which T³ and T⁴ are N can be made by reacting a compound of formula II with a compound of formula III: 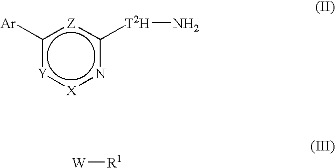
in which Ar, R¹, R², T², X, Y and Z are as defined above and W is an isocyanate or isothiocyanate group. When W is an isocyanate group the reaction is carried out in the presence of acetonitrile with heating to about 90° C. for about 12 h, followed by the addition of phosphorous oxychloride generally with heating at reflux for about 12 h, with this last step generally being repeated.

When W is an isothiocyanate group the reaction is generally heated to from 60 to 100° C. for about 1 h in a solvent such as p-xylene/N,N-dimethylacetamide after which an activating agent such as dicyclohexylcarbodiimide can be added with further heating at about 100° C. for about 1 h. The reaction can also be carried out in a solvent such as acetonitrile for about 15 h at about room temperature followed by heating with silver(I)acetate at about 150° C. for about 10 minutes in a microwave.

Compounds of formula II in which T² is N can be made by reacting a compound of formula IV: 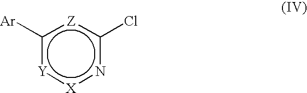
in which Ar, X, Y and Z are as defined above with hydrazine, usually as its monohydrate, in a solvent such as isopropanol at about 100° C. for about 15 h. This procedure can be repeated once or twice to improve yields.

Compounds of formula IV can be made by treating a compound of formula V with a compound of formula VI: 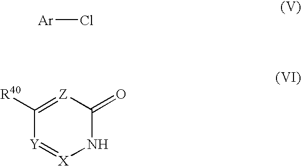
in which Ar, X, Y and Z are as defined above and R⁴⁰ is Cl or Sn(alkyl)ₛ, for example Sn(methyl)₃ or Sn(n-butyl)₃. When R⁴⁰ is Cl it can be initially converted into a group B(OH)₂ under conditions suitable for a Suzuki Coupling Reaction (for review, see for instance A. Suzuki, Pure Appl. Chem., 1991, 63, 419-422), for example, in the presence of a palladium catalyst such as tetrakis(triphenylphosphine)palladium (0), (1,1′-bis(diphenylphosphino)ferrocene)dichloropalladium or dichloro-(1,4-bis(diphenylphosphino)butane)palladium, in a suitable solvent such as an ether, for example, dimethoxyethane or dioxane or an aromatic hydrocarbon, for example toluene, at an elevated temperature and in the presence of a base such as sodium carbonate. Where R⁴⁰ is Sn(alkyl)₃, the reaction is conveniently effected under conditions suitable for a Stille Coupling Reaction (for review, see for instance J. K Stille, Angew. Chem. Int. Ed., 1986, 25, 508-524), for example, in the presence of a palladium catalyst such as tetrakis(triphenylphosphine)palladium (0) or bis(triphenylphosphine)palladium (II) chloride, in a suitable solvent such as an ether, for example dioxane, or an aromatic hydrocarbon, for example, toluene, at an elevated temperature, and in the presence of catalysts such as LiCl and CuI.

The resulting compound can be converted to the desired chloride IV by reacting with phosphorous oxychloride at about 100° C. for about 1 h.

Alternatively compounds of formula IV can be made by reacting a compound of formula ArH with a compound of formula X: 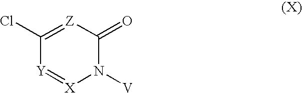
in which X, Y and Z are as defined above and V is a protecting group such as tetrahydropyranyl. The reaction is generally carried in the presence of a strong base such as BuLi, in the presence of zinc chloride and catalyst such as Pd(PPh₃)₄ in a solvent such as tetrahydrofuran between about −78° C. and room temperature for about 2 h. The resulting product can be deprotected using phosphorous oxychloride with heating to about 90° C. for about 10 min.

Compounds of formula II in which T² is C(CH₂)ₙR² can be made by reacting a compound of formula VII: 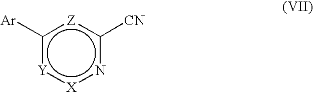
in which n, Ar, X, Y and Z are as defined above with ammonia in a hydrogenating environment, such as H₂/Pd/C, generally in a solvent such as methanol at about room temperature for about 1 h.

The nitrile of formula VII can be made by reacting the corresponding amide with a dehydrating agent such as Burgess reagent for up to 6 h in a solvent such as dichloromethane. This amide can be made from the corresponding carboxylic acid ester which is reacted with ammonia in a solvent such as methanol for about 3 h.

This carboxylic acid ethyl ester can be made from the corresponding compound of formula IV under an atmosphere of carbon monoxide in ethanol in the presence of a palladium catalyst such as Pd(dppf)Cl₂.CHCl₃ and a base such as sodium acetate at about 90° C. for about 2 h.

In an alternative route, compounds of formula I can be made by reacting a compound of formula VIII with a compound of formula IX: 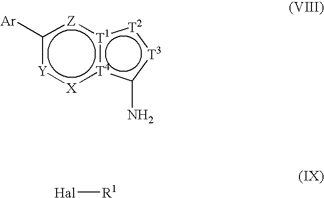
in which T¹, T², T³, T⁴, X, Y, Z, Ar and R¹ are as defined above and Hal is bromine or iodine. The reaction is generally carried out in the presence of a catalyst such as tris(dibenzylidene)dipalladium together with cesium carbonate in a solvent such as 1,4-dioxane at about 100° C. for from 15 min to 18 h. The reaction is promoted using a catalyst such as xantphos.

The compound of formula VIII can be made by reducing the corresponding nitro compound with, for example, Lindlar catalyst in MeOH:EtOAc on a Parr hydrogenator under H₂ for about 30 min.

This nitro compound can be made by nitrating a compound of formula XI: 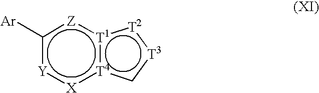
in which T¹, T², T³, T⁴, X, Y, Z and Ar are as defined above with, for example, a mixture of concentrated H₂SO₄ and fuming HNO₃ for about 30 min at about 0° C.

Compounds of formula XI in which T² and T⁴ are N and Ts is C(CH₂)ₙR² can be made by reacting a compound of formula XVII with bromoacetaldehyde or chloroacetaldehyde in a solvent such as ethanol in the presence of a mild base such as sodium hydrogencarbonate at about reflux for about 18 h. Bromoacetaldehyde can be made in situ by reacting bromoacetaldehydedimethylacetal with an acid such as hydrobromic acid in a solvent such as water.

The compound of formula XI can also be made by reacting a compound of formula V with a compound of formula XII:

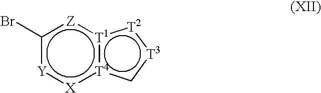

in which X, Y, Z, T¹, T², T³ and T⁴ are as defined above by a Suzuki reaction as described above, for example using bispinacolatodiborane.

Compounds of formula XII in which T¹=T²=X=N, T³=C(CH₂)R² and Y=Z=C(CH₂)ₙR³ can be made by reacting a compound of formula XIII with a compound of formula XIV: 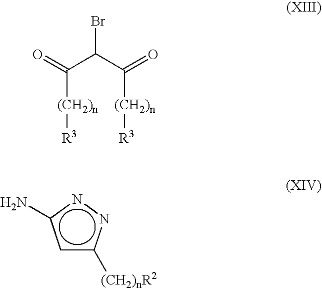
in which n, R² and R³ are as defined above, in the presence of acetic acid and in a solvent such as ethanol for about 4 h at reflux.

Compounds of formula XI can also be made by ring-closing a compound of formula II with, for example, formic acid at about 80° C. for about 30 min.

Compounds of formula VIII in which T²=T³=T⁴=N can be made by reacting a compound of formula IV with thiosemicarbazide generally in glacial acetic acid at about 135° C. for about 12 h.

An alternative route to producing compounds of formula IV in which X=N, Y=CCl and Z=CH is provided by reacting a compound of formula XV: 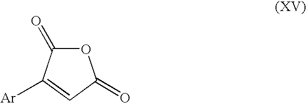
in which Ar is as defined above successively with hydrazine monohydrate and phosphorous oxychloride. The former reaction is generally carried out in glacial acetic acid with the gradual addition of concentrated sulphuric acid followed by heating to about 125° C. for about 3 h.

The compound of formula XV can be made by reacting a compound of formula XVI: 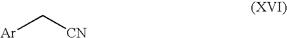
in which Ar is as defined above with glyoxylic acid monohydrate in a solvent such as methanol in the presence of a base such as potassium carbonate for about 15 h at about room temperature, followed by reacting with a mixture of formic acid and sulphuric acid generally at reflux for about 3 h.

Compounds of formula XI in which T²=T⁴=N and T³=CH can be made by reacting a compound of formula XVII: 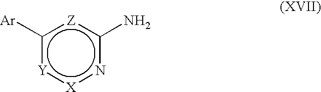
in which Ar, X, Y and Z are as defined above with chloroacetaldehyde generally in a solvent such as ethanol in the presence of a base such as sodium bicarbonate at reflux for about 18 h.

The compound of formula XVII can be made by reducing a compound of formula II in which T² is N for example with Raney Nickel under H₂ at about room temperature for about 48 h. Compounds of formula XVII can also be made by reacting a compound of formula XVIII with ammonia generally in a solvent such as water in a microwave at about 140° C. for about 30 minutes.

In an alternative method compounds of formula II can be made by reacting a compound of formula XVIII: 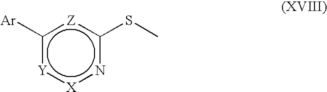
in which Ar, X, Y and Z are as defined above with hydrazine monohydrate in a solvent such as ethanol at reflux for about 16 h.

Compounds of formula XVIII in which X=Z=N and Y=CH can be made by reacting a compound of formula XIX: 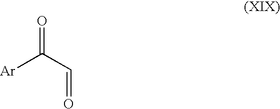
in which Ar is as defined above with aminomethanehydrazonathionate, generally as the hydroiodide salt, in a solvent such as water between about 0° C. and room temperature for about 1 h.

Compounds of formula I can also be made by reacting a compound of formula XX with a compound of formula XXI: 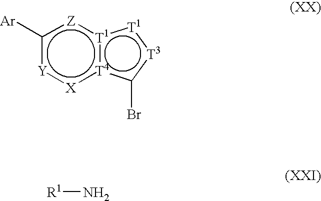
wherein T¹, T², T³, T⁴, X, Y, Z, Ar and R¹ are as defined above. The reaction is generally carried out in a solvent such as dioxane in the presence of an acid catalyst such as hydrobromic acid for about 15 min in a microwave.

The compound of formula XX can be made by brominating a compound of formula XI, for example using bromine in the presence of a buffered solution such as a mixture of acetic acid and sodium acetate at about 120° C. for about 2 h.

Compounds of formula I can be converted to other compounds of formula I by methods known in the art. Indeed, any of the intermediates can be functionalised by conventional methods. For example, compounds having an R³ group which is chlorine can be converted to compounds where that R³ group is hydrogen by reacting with ammonium formate in the presence of a catalyst such as Pd/C in a solvent such as anhydrous ethanol at about 80° C. for about 15 h.

Compounds in which Ar or Ar¹ is substituted by bromine can be converted into compounds where Ar or Ar¹ is substituted by an aromatic group by performing the appropriate Stille Compling Reaction as described above. Compounds having an acetyl group can be reacted with a methylating agent such as methyl magnesium bromide in a solvent such as tetrahydrofuran at a temperature of from −40° C. to 0° C. for about 15 h to produce the 2-hydroxyprop-2-yl analogue. Compounds in which the nitrogen atom of a pyridine moiety is oxidized can be made by reacting with, for example, oxone in a solvent such as chloroform in the presence of a catalyst such as aluminium oxide generally at reflux for about 18 h.

Compounds of formula I in which R² is H can be brominated to compounds of formula I in which R² is Br by reacting with a brominating agent such as N-bromosuccinimide in a solvent such as dichloromethane for about 5 min at about room temperature. This compound can undergo Suzuki Coupling Reactions to compounds of formula I in which R² is an aromatic group. The bromine atom can be replaced by a cyano group by reacting with zinc cyanide in the presence of a catalyst such as zinc powder and a coupling agent such as [1,1′-bis(diphenylphosphino)ferrocene]dichloropalladium(II)dichloromethane complex in a solvent such as N,N-dimethylacetamide at about 160° C. for about 20 min in a microwave. The cyano group can be converted to a formamide residue by hydrolysing with, for example, concentrated hydrochloric acid at about 80° C. for about 20 min. Compounds in which n in (CH₂)ₙR² is one and where R² is bound to the methyl group via a nitrogen atom, can be made by reacting a compound of formula I in which R² is hydrogen with formaldehyde and the nitrogen containing moiety, such as morpholine or dimethylamine, in the presence of an acid, such as acetic acid, in a solvent such as water at about room temperature for from 20 to 24 h. Compounds of formula I in which R² is carboxy can be made from compounds of formula I in which R² is bromine by reacting with carbon monoxide in ethanol in the presence of sodium acetate and a coupling agent such as [1,1′-bis(diphenylphosphino)ferrocene]dichloropalladium(II)dichloromethane complex at about reflux for about 3 h followed by hydrolysing the ester for example in a mixture of methanol, water and tetrahydrofuran in the presence of a base such as lithium hydroxide at about room temperature for about 24 h.

Intermediates for which no preparation is described above are commercially available or can be made from commercially available compounds by methods known in the art. The preparation of some of these intermediates is provided in the Descriptions and Examples.

During any of the above synthetic sequences it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene and P. G. M. Wuts, Protective Groups in Organic Synthesis, John Wiley & Sons, 1991. The protecting groups may be removed at a convenient subsequent stage using methods known from the art.

The following Examples serve to illustrate the preparation of compounds of the present invention.

### Common Intermediates

### Description 1

### 3-Chloro-5-(3-methyl-2-pyridyl)pyridazine

To a mixture of 5-chloropyridazin-3-one (0.135 g, 1 mmol) and 2-(tri-n-butylstannyl)-3-methylpyridine (0.42 g, 1.1 mmol) in anhydrous 1,4-dioxane (2 ml) was added tetrakis(triphenylphosphine)palladium (0) (0.06 g, 0.051 mmol), copper(I)iodide (20 mg, 0.1 mmol) and lithium chloride (0.13 g, 3.1 mmol) and the mixture was irradiated in a Smith microwave reactor at 160° C. for 15 min. The mixture was allowed to cool to room temperature and poured onto a mixture of ethyl acetate/water (10 ml/5 ml). The phases were separated and the aqueous phase was extracted two times more with ethyl acetate. The combined organic layers were washed with brine, dried over sodium sulfate and adsorbed onto silica gel. Purification by flash chromatography (ethyl acetate) gave 5-(3-methyl-2-pyridyl)pyridazin-3-one (0.13 g, 69%) as a yellow solid, MS: (ES (M+1)) 188. The pyridazinone (0.64 g, 3.4 mmol) was suspended in phosphorous oxychloride (5 ml, 54 mmol) and the mixture was heated at 100° C. for 1 h. After cooling to room temperature the homogeneous dark solution was evaporated under reduced pressure and repartitioned between chloroform and water (60 ml each). The pH was adjusted to 8 by portionwise addition of saturated aqueous sodium carbonate solution and the phases were separated. After two further extractions the combined organic extracts were washed with water and brine and dried over sodium sulfate. After filtration the compound was adsorbed onto silica gel and purified by flash column (50% ethyl acetate-iso-hexane) to yield the title compound (0.38 g, 54%), MS: (ES (M+1)) 205/207.

¹H NMR (500 MHz, DMSO) δ 2.44 (3H, s), 7.47 (1H, dd, J=7.6 and 4.1), 7.85 (1H, d, J=7.6), 8.16 (1H, s), 8.59 (1H, d, J=4.1), 9.48 (1H, s) ppm.

### Description 2

### 3-Chloro-5-(3-trifluoromethyl-2-pyridyl)pyridazine

To a mixture of 5-chloropyridazin-3-one (8.6 g, 62.9 mmol) and bis(pinacolato)diboron (16.8 g, 66.2 mmol) in anhydrous 1,4-dioxane (100 ml) was added bis(diphenylphosphino)ferrocenylpalladiumdichloride (2.3 g, 3.1 mmol) and potassium acetate (18.5 g, 188.5 mmol) and nitrogen was bubbled through the mixture for 10 min. The mixture was heated at 100° C. for 15 h, allowed to cool to room temperature and a mixture of 2-chloro-3-(trifluoromethyl)pyridine (10.9 g, 60 mmol) and bis(diphenylphosphino)ferrocenylpalladiumdichloride (2.3 g, 3.1 mmol) followed by 2M sodium carbonate (100 ml) was added to the black mixture and nitrogen was bubbled through for 10 min. The resulting mixture was heated at 100° C. for 15 h, allowed to cool to room temperature and poured into a mixture of ethyl acetate/ethanol/water (500/100/100 ml). The phases were separated and the aqueous phase was extracted two times with ethyl acetate (200 ml each). The combined organic layers were washed with brine, dried over sodium sulfate and adsorbed onto silica gel. Purification by flash chromatography (ethyl acetate) gave 5-(3-trifluoromethyl-2-pyridyl)pyridazin-3-one (4.9 g, 32%) as an off white solid, MS: (ES(M+1)) 242.

The pyridazinone (4.8 g, 20 mmol) was suspended in phosphorous oxychloride (30 ml, 322 mmol) and the mixture was heated at 100° C. for 1 h. After cooling to room temperature the homogeneous dark solution was evaporated under reduced pressure and repartitioned between chloroform and water (50 ml each). The pH was adjusted to 8 by portionwise addition of saturated aqueous sodium carbonate solution and the phases were separated. After two further extractions the combined organic extracts were washed with water and brine and dried over sodium sulfate. After filtration the compound was adsorbed onto silica gel and purified by flash column (50% ethyl acetate-iso-hexane) to yield the title compound (3.9 g, 75%), MS: (ES (M+1)) 260/262.

¹H NMR (360 MHz, DMSO) δ 7.85 (1H, dd, J=7.5 and 4.5), 8.16 (1H, d, J=1.5), 8.45 (1H, d, J=7.5), 9.02 (1H, d, J=4.5), 9.43 (1H, d, J=1.5) ppm.

### Description 3

### 3-Chloro-5-(2-methoxyphenyl)pyridazine

Using a procedure analogous to that given in Description 2:

(1.5 g, 25%) as a colourless solid, MS: (ES (M+1)) 221/223.

### Description 4

### 3-Amino-7-(3-trifluoromethyl-2-1)pyridyl)-1,2,4-triazolo[4,3-b]pyridazine

A mixture of Description 2 (1.43 g, 5.5 mmol) and thiosemicarbazide (0.51 g, 5.6 mmol) in glacial acetic acid (10 ml) was heated at 135° C. for 12 h. After cooling to room temperature the dark mixture was concentrated under reduced pressure and repartitioned between chloroform and water (150 ml/50 ml). After two further extractions the combined organic extracts were washed with brine and dried over sodium sulfate. After filtration the compound was adsorbed onto silica gel and purified by flash column (10% ethanol—ethyl acetate) to yield the title compound (0.67 g, 44%) as a yellow solid, MS: (ES(M+1)) 281.

¹H NMR (360 MHz, DMSO) δ 6.74 (2H, s), 7.77 (1H, dd, J=7.9 and 4.9), 8.12 (1H, d, J=1.3), 8.41 (1H, d, J=7.9), 8.54 (1H, d, J=1.3), 9.00 (1H, d, J=4.9) ppm.

### Description 5

### 6-Chloro-3-hydrazino-5-(4-trifluoromethphenyl)pyridazine

4-Trifluoromethylphenylacetonitrile (38.9 g, 210 mmol) was dissolved in dry methanol under nitrogen. Glyoxylic acid monohydrate (29 g, 315 mmol) was added followed by potassium carbonate (74 g, 535 mmol) and the resulting mixture was stirred for 15 h at room temperature. The resulting solid was filtered, washed with dichloromethane and dried on the sinter to yield an off-white solid which was added at room temperature to a solution of conc. sulfuric acid (30 ml) and formic acid (400 ml). The resulting mixture was then heated with stirring at 110° C. for 3 h, allowed to cool to room temperature and concentrated under vacuum to ⅔ of the initial volume. It was then poured ice-water (1000 ml) and the resulting solid was filtered off, washed with water and dried on the sinter to yield 35 g of 3-(4-trifluoromethylphenyl)maleic anhydride as an off-white solid.

The crude anhydride (35 g) was suspended in water (290 ml) and glacial acetic acid (145 ml) was added followed by a solution of hydrazine hydrate (7 ml, 144 mmol) in water (21 ml). After thorough mixing conc. sulfuric acid was added in small portions with external water cooling and the mixture was heated while stirring at 125° C. for 3 h. After cooling to room temperature the solid was filtered off, washed with water until the pH was neutral and dried on the sinter to yield a grey solid. Phosphorous oxychloride (200 ml, 2.1 mol) was added to the solid and the mixture was heated at 120° C. for 2 h. After cooling to room temperature the homogeneous dark solution was concentrated under reduced pressure to ½ of its original volume and poured into water (800 ml) while stirring vigorously. The resulting solid was filtered off, washed with water and dried on the sinter to yield a grey solid which was recrystallised from toluene/iso-hexane (1:1) to yield the title compound as a yellow solid (8.2 g, 13%). ¹H NMR (360 MHz, DMSO) δ 7.86 (2H, d, J 8.0), 7.95 (2H, d, J 8.0), 8.22 (1H, s).

### Description 6

### 2,6-Dichloro-4-(3-methyl-2-pyridyl)pyridine

To a mixture of 2,6-dichloropyridine (3.28 g, 22.2 mmol) and bis(pinacolato)diboron (6.2 g, 24.4 mmol) was added 1,10-phenanthroline (0.24 g, 1.3 mmol) and chloro-1,5-cyclooctadiene iridium (I) dimer (0.44 g, 0.66 mmol) under nitrogen followed by anhydrous 1,2-dichloroethane. Nitrogen was bubbled through the mixture for 5 min and the reaction was then heated with stirring at 100° C. for 15 h under an atmosphere of nitrogen. The mixture was allowed to cool to room temperature, poured onto diethylether/4N sodium hydroxide (50 ml/200 ml) and the phases separated. The aqueous phase was acidified with 6N hydrochloric acid and the resulting solid was filtered, washed with water and dried on the sinter to yield pinacol 2,6-dichloropyridine-4-boronate (3.5 g, 58%) as a grey solid, MS: (ES (M+1)) 274/276.

¹H NMR (360 MHz, DMSO) δ 1.30 (12H, s), 7.57 (2H, s) ppm.

To a mixture of the boronate (3.7 g, 13.5 mmol) and 2-bromo-3-methylpyridine (2.3 g, 13.4 mmol) in anhydrous dioxane (25 ml) was added (1,1′-bis(diphenylphosphino)ferrocene)dichloropalladium (0.45 g, 0.61 mmol) and saturated aqueous sodium carbonate solution (14 ml). Nitrogen was bubbled through the mixture for 5 min and the reaction was then heated with stirring at 100° C. for 4 h under an atmosphere of nitrogen. The mixture was allowed to cool to room temperature, poured onto water (200 ml) and the pH adjusted to pH=7 by addition of 1N HCl. The solid was filtered, washed with water and dried to yield the title compound (3.2 g, quant.) as a brown solid, MS: (ES (M+1)) 239/241.

¹H NM (360 MHz, DMSO) δ 2.38 (3H, 8), 7.44 (1H, dd, J 7.6 and 4.7), 7.76 (2H, 9), 7.81 (1H, d, J 7.6), 8.55 (1H, d, J 4.7) ppm.

### Description 7

### 2,6-Dichloro-4-(3-trifluoromethyl-2-pyridyl)pyridine

Using a procedure analogous to that given in Description 6, using 2-bromo-3-trifluoromethylpyridine:

(0.5 g, 10%) as a colourless solid, MS: (ES (M+1)) 293/295.

¹H NMR (400 MHz, CDCl₃) δ 7.42 (2H, s), 7.56 (1H, m), 8.15 (1H, dd J=7.7 and 0.9), 8.89 (1H, d, J 5.0) ppm.

### Description 8

### 2,6-Dichloro-4-(2-methoxyphenyl pyridine

Using a procedure analogous to that given in Description 6, using 2-bromoanisole;

(4.7 g, 65%) as a grey solid, MS: (ES (M+1)) 254/256.

¹H NMR (360 MHz, DMSO) δ 3.84 (3H, s), 7.07-7.11 (1H, m), 7.19 (1H, d, J=8.7), 7.47-7.49 (2H, m), 7.70 (1H, s) ppm.

### Description 9

### 5-(3-Trifluoromethylpyridin-2-yl)pyridazine-3-carboxylic acid ethyl ester

To a solution of Description 2 (0.50 g, 1.93 mmol) in ethanol in a 3-neck flask equipped with a condenser and a bubbler was added sodium acetate (0.32 g, 3.86 mmol). Nitrogen was bubbled through the resulting solution for 10 min. Pd(dppf)Cl₂.CHCl₃ (0.10 g, 0.14 mmol) was added and the reaction flushed with carbon monoxide. After 5 min of rapid CO bubbling the orange solution had darkened. The gas flow rate was reduced and the reaction heated to 90° C. After 2 h the starting material had been consumed and the solution was flushed with nitrogen. The reaction was condensed, partitioned between pH 7 phosphate buffer and ethyl acetate and the aqueous layer washed again with ethyl acetate. The organic layers were combined, dried over MgSO₄ and the crude product purified by flash column chromatography, eluting with 50% to 25% hexane in ethyl acetate to give the ethyl ester (0.37 g, 66%). m/z(ES⁺) 297 (M+H⁺). ¹H NMR (400 MHz, CDCl₃) 1.51 (3H, t, J 7.1), 4.59 (2H, q, J 7.1), 7.63 (1H, m), 8.20 (1H, dd, J 8.1, 0.8), 8.38 (1H, d, J 2.1), 8.96 (1H, d, J 0.7), 9.51 (1H, d, J 2.1).

### Description 10

### 5-(3-Trifluoromethylpyridin-2-yl)pyridazine-3-carboxylic acid amide

To Description 9 (150 mg) was added a solution of ammonia in methanol (2 M, 10 ml) and the reaction stirred for 3 h. The reaction was condensed to yield the desired amide (140 mg, 100%). m/z (ES⁺) 269 (M+H⁺). ¹H NMR (400 MHz, CDCl₃) 5.96 (1H, s), 7.61 (1H, ddd, J 7.8, 4.7, 0.9), 8.07 (1H, s), 8.19 (1H, dd, J 7.9, 1.0), 8.50 (1H, d, J 2.2), 8.96 (1H, d, J 5.0), 9.47 (1H, d, J 2.2).

### Description 11

### 5-[3-Trifluoromethylpyridin-2-yl]pyridazin-3-amine

Raney Nickel (50% aq. suspension, 2 ml) was added to a solution of 3-hydrazino-5-[3-trifluoromethylpyridin-2-yl]pyridazine (from Example 1; 1.10 g, 4.31 mmol) in ethanol (100 ml). The mixture was then stirred under a balloon of hydrogen gas for 48 h. The catalyst was then filtered off on a glass fibre pad, washing the solid thoroughly with ethanol. The filtrate was evaporated and the residue was then purified using a strong cation exchange (SCX) ion exchange cartridge washing away non-basic impurities with methanol, then eluting with 2M methanolic ammonia solution. Evaporation of the basic fraction gave the title compound as a red-brown solid (573 mg). ¹H NMR (400 MHz, DMSO) δ 8.97 (1H, br. d, J 5), 8.48 (1H, d, J 2), 8.37 (1H, d, J 8), 7.75 (1H, dd, J 8, 5), 6.82 (1H, d, J 2), 6.60 (2H, br. s); m/z (ES⁺) 241 (M+H⁺).

### Description 12

### 7-[3-Trifluoromethylpyridin-2-yl]imidazo[1,2-b]pyridazine

Description 11 (570 mg, 2.38 mmol) was dissolved in ethanol (10 ml). Sodium bicarbonate (400 mg, 4.75 mmol) was then added followed by chloroacetaldehyde (45% aq. solution, 450 μl, ca. 3.25 mmol) and the reaction mixture was heated at reflux for 18 h. After cooling to room temperature, flash silica was added, the solvent removed and the residue purified by flash column chromatography (eluant 1:19 MeOH—CH₂Cl₂) to give the title compound. ¹H NMR (400 MHz, DMSO) δ 9.01 (1H, d, J 5), 8.68 (1H, d, J 2), 8.44 (1H, br. s), 8.42 (1H, d, J 8), 8.25 (1H, br. s), 7.93 (1H, s) 7.78 (1H, dd, J 8, 5); m/z (ES+) 265 (M+H⁺).

### Description 13

### 3-Nitro-7-[3-trifluoromethylpyridin-2-yl]imidazo[1,2-b]pyridazine

Description 12 (337 mg, 1.28 mmol) was dissolved in conc. suluric acid (3 ml) at 0° C. A nitrating mixture of conc. sulfuric acid and fuming nitric acid (1:1, 2 ml) was then added dropwise over 10 min. The mixture was then allowed to warm to room temperature and stir for 20 h before pouring into ice-water (150 ml). The mixture was made basic by addition of 33% aqueous ammonia solution, then extracted with ethyl acetate (3×30 ml). The combined organic layers were dried (Na₂SO₄) and evaporated and the residue purified by flash column chromatography (eluant 1:19 MeOH—CH₂Cl₂) to give the title compound (240 mg) as a colourless solid. ¹H NMR (400 MHz, DMSO) δ 9.14 (1H, d, J 2), 9.06 (1H, d, J 5), 8.93 (1H, 8), 8.2 (1H, d, J 2), 8.47 (1H, d, J 8), 7.84 (1H, dd, J 8, 5); m/z (ES⁺) 310 (M+H⁺).

### Description 14

### 7-[3-Trifluoromethylpyridin-2-yl]imidazo[1,2-b]pyridazin-3-amine

Lindlar catalyst (100 mg) slurried in ethanol (1 ml) was added to a solution of Description 13 (170 mg, 0.55 mmol) in an ethanol—ethyl acetate mixture (1:1, 10 ml). The reaction mixture was then stirred under a balloon of hydrogen gas at room temperature for 5 h. The mixture was then filtered, washing the catalyst with ethanol (5 ml) and the filtrate was then evaporated. Addition of toluene (5 ml) to the residue and re-evaporation gave the title compound (153 mg) as a red oil which was free of ethanol and used without further purification. ¹H N (500 MHz, DMSO) δ 8.98 (1H, d, J 5), 8.54 (1H, s), 8.38 (1H, d, J 8), 7.96 (1H, s), 7.71 (1H, dd, J 8, 5), 7.21 (1H, s), 5.74 (2H, s); m/z (ES⁺) 280 (M+H⁺).

### Description 15

### 2-(3-Methylpyridine)glyoxaldehyde dimethylacetal

To a solution of 2-bromo-3-methylpyridine (1 g, 5.8 mmol) in tetrahydrofuran (15 ml) at −78° C. was added n-butyllithium (1.6 M in hexanes, 3.81 ml, 6.1 mmol) in a dropwise fashion resulting in a dark red solution. After 5 min 1-piperidineglyoxaldehyde dimethylacetal (1.56 g, 6.96 mmol) in tetrahydrofuran (10 ml) was cannulated into the reaction mixture. The reaction mixture became pale yellow. After 30 min, the reaction was quenched with saturated ammonium chloride, then extracted three times with ethyl acetate and dried over sodium sulfate. After filtration the compound was adsorbed onto silica gel and purified by flash column (30% ethyl acetate in hexanes) to yield the title compound (655.9 mg, 59%) as a yellow oil.

¹H NMR (400 MHz, DMSO) δ 8.52 (1H, app d, J 4.1), 7.62-7.60 (1H, m), 7.34 (1H, dd J 8.0, 4.8), 6.0 (1H, s), 3.49 (6H, s), 2.57 (3H, s) ppm

### Description 16

### 5-(3-Methylpyridin-2-yl)-3-methylsulfanyl[1,2,4]triazine

To conc. H₂SO₄ (3 g) at 0° C. was added dropwise Description 15 (1 g, 5 mmol). The reaction was allowed to warm to room temperature and was stirred for two days. Ice and water were then slowly added and the reaction mixture was carefully neutralized by addition of NaHCO₃. This solution containing the 2-(3-methylpyridine)glyoxaldehyde was used without further purification. The aqueous solution of 2-(3-methylpyridine)glyoxaldehyde (assumed to be 5 mmol) was cooled to 0° C., and methyl aminomethanehydrazonothioate hydroiodide (525 mg, 5 mmol) in water (10 ml) was added. The reaction was allowed to warm to room temperature and after 1 h was filtered to give the title compound (228 mg, 21%, two steps)

¹H NMR (400 MHz, DMSO) δ 9.67 (1H, s), 8.66 (1H, dd J 4.2, 1.2), 7.88 (1H, d, J 7.6), 7.55 (1H, dd, J 8.0, 4.8), 2.66 (3H, s), 2.66 (3H, s) ppm; MS (MH⁺) 219.

### Description 17

### 5-(3-Methylpyridin-2-yl)[1,2,4]triazin-3-ylhydrazine

To a solution of Description 16 (228 mg, 1.0 mmol) in ethanol was added hydrazine hydrate. The mixture was heated to reflux and stirred for 16 h, then allowed to cool and the solvent removed in vacuo. The yellow oil was preadsorbed onto silica gel (ethylacetate) then purified by column chromatography (80% ethyl acetate in hexanes) to give title compound (88.9 mg, 44%) as well as recovered starting material (101 mg, 46%).

¹H NMR (400 MHz, DMSO) δ 9.09 (1H, s), 8.79 (1H, bs), 8.59 (1H, dd J 4.4, 1.2), 7.83-7.81 (1H, m), 7.55 (1H, dd, J 8.0, 4.8), 2.61 (3H, s) ppm; MS (MH⁺) 203.

### Description 18

### 1-{5-[3-trifluoromethylpyridin-2-yl]pyridazin-3-yl}ethanone

Description 2 (1.00 g, 3.86 mmol), tributyl(1-ethoxyvinyl)tin (1.56 ml, 4.63 mmol), palladium tetrakis triphenylphosphine (0.22 g, 0.19 mmol), copper (I) iodide (73 mg, 0.39 mmol) and lithium chloride (0.49 g, 11.6 mmol) were combined in dioxan (30 ml) and the reaction was heated at 110° C. for 14 h. The reaction was cooled and hydrochloric acid (2N, 20 ml) was added and the reaction stirred at room temperature for 2 h. The reaction was diluted with saturated ammonium chloride solution and extracted with dichloromethane. The organic fraction was washed with saturated potassium fluoride solution, dried over magnesium sulfate and condensed. The crude product was purified by flash chromatography (25% ethyl acetate in hexanes) to give the title compound as a colourless oil (0.56 g, 54%). m/z (ES⁺) 268 (M+H⁺). ¹H NMR (360 MHz, CDCl₃) 9.50 (1H, s), 8.94 (1H, d, J 4.8), 8.30 (1H, s), 8.19 (1H, d, J 8.0), 7.61 (1H, t, J 6.4), 2.97 (3H, s).

### Description 19

### (1-{5-[3-trifluoromethylpyridin-2-yl]pyridazin-8-yl}ethyl)amine

To a solution of Description 18 (28 mg, 0.11 mmol) in formamide (0.23 ml, 5.7 mmol) at 140° C. was added formic acid (0.12 ml, 3.15 mmol) and the resulting reaction was stirred at this temperature for 3 h. The reaction was cooled and hydrochloric acid was added (2N, 0.5 ml). After stirring for 3 h at 100° C. the formamide intermediate remained and additional hydrochloric acid was added (4N, 0.6 ml) and the reaction heated for 24 h at 80° C. Then concentrated hydrochloric acid (˜12 M, 1 drop) was added and the reaction was complete within 1 h. The reaction was condensed, basified with sodium hydroxide (2 M), extracted with dichloromethane (4×10 ml), dried over sodium sulfate and condensed. The crude product was loaded on to a strong cation exchange (SCX) cartridge, washed with methanol and the product eluted with methanolic ammonia (2 M). The product containing fractions were condensed and azeotroped with ethanol to give the title compound which was used without further purification (16 mg).

### Description 20

### 5-(3-Methylpyridin-2-yl)pyridazine-3-carboxamide

Prepared from Description 1 according to the procedures of Description 9 and 10 respectively. ¹H NMR (360 MHz, DMSO) δ 9.66 (1H, d, J=2.2 Hz), 8.66 (1H, s), 8.62 (1H, d, J=4.5 Hz), 8.36 (1H, d, J=2.2 Hz), 8.01 (1H, s), 7.86 (1H, d, J=8 Hz), 7.47 (1H, dd, J=4.5, 8 Hz), 2.45 (3H, s).

### Description 21

### 5-(3-Methylpyridin-2-yl)pyridazine-3-carbonitrile

To a suspension of Description 20 (1.58 g, 7.38 mmol) in dichloromethane at room temperature, was added (methoxycarbonylsulfamoyl)triethylammonium hydroxide inner salt (Burgess reagent) (3.5 g, 14.7 mmol) in 3 roughly equal portions over a 1 h period. The mixture was stirred at room temperature for a further 2 h, then flash silica (ca. 30 ml) was added and the solvent evaporated. Purification by flash column chromatography (75% EtOAc in isohexane then EtOAc) gave the title compound (1.34 g). ¹H NMR (360 MHz, CDCl₃) δ 9.64 (1H, d, J=2.2 Hz), 8.64 (1H, d, J=4.5 Hz), 8.12 (1H, d, J=2.2 Hz), 7.72 (1H, d, J=8 Hz), 7.38 (1H, dd, J=4.5, 8 Hz), 2.50 (3H, s).

### Description 22

### 2-Methyl-7-[3-trifluoromethylpyridin-2-yl]imidazo[1,2-b]pyridazine

Prepared from Description 11 and chloroacetone according to the procedure of Description 12. ¹H NMR (400 MHz, DMSO) δ 9.00 (1H, dd, J=0.8, 4.8 Hz), 8.59 (1H, d, J=2 Hz), 8.41 (1H, dd, J=1.3, 8.1 Hz), 8.20 (1H, s), 8.10 (1H, d, J=2 Hz), 7.78-7.74 (1H, m), 2.44 (3H, s) ppm.

### Description 23

### 3-chloro-5-(3-chloropyridin-2-yl)-pyridazine

Prepared from Description 46 according to the procedures of Description 2. ¹H NMR (400 MHz, CDCl₃) 9.59 (1H, d, J 1.8), 8.69 (1H, dd, J 1.4, 4.6), 7.99 (1H, d, J 1.8), 7.90 (1H, dd, J 1.4, 8.2), 7.46-7.40 (1H, m) ppm.

### Description 24

### 7-(3-chloropyridin-2-yl)imidazo[1,2-b]pyridazin-3-amine

Prepared from Description 23 according to the procedures of Example 1 and Descriptions 11, 12, 13 and 14. m/z (ES⁺) 246, 248 (M+H⁺). ¹H NMR (500 MHz, CDCl₃) 8.79 (1H, d, J 2.0), 8.63 (1H, dd, J 1.5, 4.6), 8.38 (1H, d, J 2.0), 7.84 (1H, dd, J 1.5, 8.1), 7.35 (1H, s), 7.27 (1H, s), 4.19 (2H, s) ppm.

### Description 25

### 2-Cyano-3-trifluoromethylpyridine

To a solution of 2-chloro-3-trifluoromethylpyridine (5 g, 28.9 mmol) in dimethylformamide (40 ml) was added zinc cyanide (2 g, 17.4 mmol), zinc dust (85 mg, 1.3 mmol) and 1,1-bisdiphenylphosphino-ferrocene dichloropalladium(II) complex with dichloromethane (460 mg, 0.63 mmol). The reaction mixture was refluxed for 4 h, then allowed to cool, diluted with ethyl acetate and washed with brine. The aqueous phase was back-extracted with ethyl acetate and the combined organic phases dried over sodium sulfate, filtered and concentrated. Purification by flash column chromatography (20% ethyl acetate in hexanes) gave the title compound (4.67 g, 98%) as a yellow oil. ¹H NMR (400 MHz, CDCl₃) δ 8.59 (1H, dd J 4.4, 1.2), 8.05-8.02 (1H, m), 7.40-7.37 (1H, m) ppm.

### Description 26

### 2-Acetyl-3-trifluoromethylpyridine

To a solution of Description 25 (4.96 g, 28.8 mmol) in tetrahydrofuran (50 ml) at −10° C. was added methylmagnesium bromide (3M in tetrahydrofuran, 9.61 ml, 31.7 mmol) at such a rate so as to ensure that the internal temperature of the reaction did not exceed 20° C. The reaction was allowed to stir at room temperature for 1 h. after addition was complete. The reaction was then quenched with 2M HCl. The organic phase was separated and the aqueous phase basified by addition of sodium carbonate. The mixture was extracted three times with ethyl acetate and the combined organic phases dried over sodium sulfate, filtered and concentrated. Purification by flash column chromatography (20% ethyl acetate in hexanes) gave the title compound (4.5 g, 83%) as a colorless oil, MS: (ES (M+1)) 190. ¹H NMR (400 MHz, CDCl₃) δ 8.79 (1H, dd J 4.8, 0.8), 8.09 (1H, dd J 8.0, 0.8), 7.57-7.53 (1H, m) ppm.

### Description 27

### 2-Bromo-1-[3-trifluoromethylpyridin-2-yl]ethanone

Description 26 (5 g, 26.45 mmol) was dissolved in tetrahydrofuran (100 ml) and phenyltrimethylammoniumtribromide (19.9 g, 52.91 mmol) added. The reaction was heated to reflux and stirred for 16 h. After cooling, the reaction was filtered and adsorbed onto silica gel. Purification by flash chromatography (10% ethyl acetate/isohexane) gave the title compound (8.5 g, 93%) as a colorless oil. MS: (ES (M+1)) 268, 270.

¹H NMR (360 MHz, DMSO) δ 8.97 (1H, t, J=2.3 Hz), 8.43 (1H, dd, J=0.8, 8.1 Hz), 7.91-7.89 (1H, m), 7.84 (0H, s), 5.00 (2H, s) ppm.

### Description 28

### 3-Methylthio-5-[3-trifluoromethylpyridin-2-yl][1,2,4]-triazine

Description 27 (8.5 g, 24.7 mmol) was dissolved in acetonitrile (80 ml) and silver nitrate (5.03 g, 29.65 mmol) added. The reaction was stirred for 16 h, then filtered (washing with diethyl ether (20 ml)) and concentrated. The residue was dissolved in diethyl ether and washed with water, then dried over sodium sulfate and concentrated. The crude mixture was then dissolved in DMSO (125 ml) and a suspension of sodium acetate trihydrate (336 mg) in DMSO (125 ml) was added. After 30 min the black solution was poured into a mixture of ice and water whereupon it became yellow. Solid sodium chloride was added and the mixture extracted three times with diethyl ether, dried over sodium sulfate and concentrated. The crude glyoxal was dissolved in ethanol (200 ml), then sodium bicarbonate (4.29 g, 49.4 mmol) added. Methyl aminomethanehydrazonothioate hydroiodide (5.76 g, 24.70 mmol) was dissolved in water (40 ml) and added to the reaction mixture. The orange solution was stirred for 16 h, then quenched with water, extracted three times with ethyl acetate then dried over sodium sulfate and concentrated. Purification by column chromatography (10-30% ethyl acetate/iso-hexane) gave the title compound (3 g, 45% over three steps) as an orange solid. MS: (ES (M+1)) 273.

¹H NMR (400 MHz, DMSO) δ 9.44 (1H, s), 8.94 (1H, dd J 4.4, 0.8), 8.22 (1H, dd, J 8.0, 0.8), 7.55 (1H, dd, J 8.0, 4.8), 2.66 (3H, s), 2.68 (3H, s) ppm.

### Description 29

### 5-(3-Trifluoromethylpyridin-2-yl)[1,2,4]triazin-3-yl]hydrazine

To a solution of Description 28 (80 mg, 0.29 mmol) in isopropanol (2 ml) was added hydrazine hydrate (49 μl, 1.56 mmol). The mixture was heated to reflux and stirred for 16 h, then allowed to cool and the solvent removed in vacuo. The yellow oil was preadsorbed onto silica gel (ethylacetate) then purified by column chromatography (80% ethyl acetate in hexanes) to give title compound (63 mg, 85%). MS: (ES (M+1)) 257.

¹H NMR (400 MHz, DMSO) δ 9.12 (1H, s), 8.93 (1H, d, J 3.96), 8.21-8.17 (1H, m), 7.64-7.58 (1H, m), 7.14 (1H, bs), 4.21 (1H, bs) ppm.

### Description 30

### 5-(3-Chloropyridin-2-yl)-3-hydrazino[1,2,4]triazine

Using a procedure analogous to that given in Description 29 (but without purification), using 5-(3-chloropyridin-2-yl)-3-(methylthio)[1,2,4]triazine (obtained analogously to Descriptions 25-28), the title compound (243 mg crude) was obtained as a brown solid that was used directly in the next reaction, MS: (ES (M+1)) 223.

### Description 31

### 5-(3-Methylpyridin-2-yl)[1,2,4]triazin-3-amine

Description 16 (505 mg) was evenly split into four reactor tubes and 33% aqueous ammonia solution (3 ml) was added to each. The vessels were heated at 160° C. in the microwave reactor for 15 min. The contents of the tubes were then combined and evaporated to give the title compound (430 mg). ¹H NMR (400 MHz, DMSO) δ 9.02 (1H, s), 8.58-8.54 (1H, m), 7.81 (1H, br. d, J=7.4 Hz), 7.47 (1H, dd, J=4.6, 7.7 Hz), 7.25 (2H, s), 2.58 (3H, s).

### Description 32

### 3-(3-Methylpyridin-2-yl)imidazo[1,2-b][1,2,4]triazine

Bromoacetaldehyde dimethyl acetal (582 mg, 3.44 mmol), water (5 ml) and 48% aqueous HBr (0.52 ml, 4.60 mmol) were heated together at 95° C. for 1 h, then the mixture was cooled to room temperature. Solid sodium bicarbonate (500 mg, 6.0 mmol) was added portion-wise then ethanol (8 ml) was added. This mixture was added to a suspension of Description 31 (430 mg, 2.30 mmol) in ethanol (15 ml) and the resulting mixture heated at reflux for 18 h. After cooling to RT, the residue was purified by flash column chromatography (eluant 2.5% MeOH in CH₂Cl₂) to give the title compound (177 mg). ¹H NMR (400 MHz, DMSO) δ 9.28 (1H, s), 8.64 (1H, dd, J=1.2, 4.6 Hz), 8.43 (1H, d, J=1.3 Hz), 8.05 (1H, d, J=1.3 Hz), 7.88 (1H, br. d, J=7.8 Hz), 7.50 (1H, dd, J=4.6, 7.8 Hz), 2.72 (3H, s).

### Description 33

### 1-(3-Chloropyridin-2-yl)-2,2-dimethoxyethanone

DABCO (5.45 g, 48.5 mmol) was dissolved in ether (200 ml) at room temperature under a nitrogen atmosphere, then the solution cooled to −40° C. n-Butyllithium (1.6M in hexanes, 30.4 ml, 48.5 mmol) was added over 10 min, the reaction stirred a further 0.5 h at −40° C., then cooled to −65° C. 3-Chloropyridine (5.0 g, 44.1 mmol) was added over 10 min, the mixture stirred for 45 min, then 1-(dimethoxyacetyl)piperidine (8.24 g, 44.1 mmol) was added over 15 min, keeping the internal temperature below −60° C. Stirred 20 min, allowing the internal temperature to rise to −50° C., then the mixture was poured into saturated aqueous NH₄Cl solution (250 ml) and warmed to room temperature. Water (100 ml) was added and the mixture extracted with ethyl acetate (2×200 ml). The combined organic layers were evaporated and the residue purified by flash column chromatrography (eluant 25% EtOAc in isohexanes) to give the title compound (5.12 g, 54%). ¹H NMR (400 MHz, CDCl₃) δ 8.56 (1H, dd, J=1.4, 4.6 Hz), 7.81 (1H, dd, J 1.4, 8.2 Hz), 7.39 (1H, dd, J=4.6, 8.2 Hz), 5.81 (1H, s), 3.48 (7H, s) ppm.

### Description 34

### 3-(3-Chloropyridin-2-yl)imidazo[1,2-b][1,2,4]triazine

Prepared from Description 33 according to the procedures of Descriptions 5, 1 and 16 respectively.

¹H NMR (500 MHz; CDCl₃) δ 9.09 (1H, s), 8.67 (1H, d, J 4.6), 7.92 (1H, d, J 8.1), 7.47 (1H, dd, J 1.3 and 8.1), 7.39 (1H, dd, J 4.4 and 8.1), 6.95 (1H, dd, J 4.5 and 8.1) ppm.

### Description 35

### 3-(3-Chloropyridin-2-yl)-7-nitroimidazo[1,2-b][1,2,4]triazine

To a solution of Description 34 (190 mg, 0.8 mmol) in conc. sulfuric acid (5 ml) was added a nitrating mixture of conc. sulfuric acid (0.5 ml) and fuming nitric acid (0.5 ml). This was stirred at room temperature for 1 h, heated at 50° C. for 16 h and then heated at 80° C. for 7 h. The reaction mixture was neutralized with K₂CO₃, filtered, washed through with water and EtOAc and then the filtrate was separated. The water layer was extracted 3 times with EtOAc and the combined organic layers were dried over MgSO₄, filtered and concentrated to give the title compound (120 mg, 53%).

¹H NMR (500 MHz; CDCl3) δ 9.55 (1H, s), 8.86 (1H, s), 8.74 (1H, dd, J 1.4 and 4.5), 7.98 (1H, dd, J 1.4 and 8.2), 7.49-7.47 (1H, m).

### Description 36

### 3-(3-Chloropyridin-2-yl)imidazo[1,2-b][1,2,4]triazin-7-amine

To a solution of Description 35 (120 mg, 0.4 mmol) in a mixture of ethanol (5 ml) and ethyl acetate (5 ml) was added Lindlar catalyst (115 mg). The reaction mixture was stirred under a balloon of hydrogen at room temperature for 24 h. A further portion of Lindlar catalyst (60 mg) was added to the reaction mixture and stirred at room temperature under a balloon of hydrogen for a further 24 h. The reaction mixture was filtered and concentrated. The residue was purified by flash column chromatography over silica, eluant system 2% MeOH in DCM to give the title compound (80 mg, 75%).

¹H NMR (500 MHz; DMSO) δ 8.98 (1H, s), 8.70 (1H, dd, J 1.4 and 4.6), 8.12 (1H, dd, J 1.4 and 8.1), 7.54 (1H, dd, J 4.5 and 8.1), 7.49 (1H, s), 6.14 (2H, s).

### Description 37

### 5-[3-Trifluoromethylpyridin-2-yl][1,2,4]triazin-3-amine

Description 28 (300 mg, 1.10 mmol) was dissolved in aqueous ammonia (4 ml) and heated in the microwave at 140° C. for 30 min. The precipitate was collected by filtration and dried under vacuum to yield the title compound (170 mg, 64%) as a pale brown solid. MS: (ES (M+1)) 242.

¹H NMR (400 MHz, DMSO) δ 8.99 (1H, t, J=2.8 Hz), 8.86 (1H, s), 8.43 (1H, dd, J=1.0, 8.0 Hz), 7.84-7.82 (1H, m), 7.42 (2H, s) ppm.

### Description 38

### 3-[3-Trifluoromethylpyridin-2-yl]imidazo[1,2-b][1,2,4]triazine

Bromoacetaldehydedimethylacetal (0.8 ml, 6.71 mmol) was taken up in water (1.1 ml) and HBr (48% aq., 1.1 ml) was added. The mixture was heated to reflux for 1 h, then allowed to cool. Sodium carbonate (856 mg) and ethanol (20 ml) were then introduced and this mixture was added to Description 37 (0.851 g, 3.53 mmol) in ethanol (20 ml). The reaction was then heated to reflux and stirred for 16 h, then allowed to cool, concentrated, preadsorbed onto silica gel and purified by column chromatography (50% ethyl acetate/iso-hexane) to yield the title compound (295 mg, 31%) as a pale yellow solid.

MS: (ES (M+1)) 266.

¹H NMR (400 MHz, DMSO) δ 9.11 (1H, s), 8.98 (1H, d, J=4.5 Hz), 8.43 (2H, t, J=9.4 Hz), 8.07 (1H, s), 7.78 (1H, dd, J=4.9, 8.0 Hz) ppm.

### Description 39

### 7-Nitro-3-[3-trifluoromethylpyridin-2-yl]imidazol-[1,2-b][1,2,4]triazine

Description 38 (295 mg, 1.09 mmol) was dissolved in conc. H₂SO₄ (3.4 ml) and a mixture of conc. H₂SO₄ (1.68 ml) and fuming HNO₃ (1.68 ml) was added at 0° C. The mixture was allowed to warm to room temperature and then heated to 65° C. for 2 days. The reaction was then poured into a mixture of ice and water and basified with sodium bicarbonate. This was extracted three times with ethyl acetate, dried over sodium sulfate and concentrated to give the title compound (285 mg, 84%) as a yellow solid. MS: (ES (M+1)) 311.

¹H NMR (400 MHz, DMSO) δ 9.63 (1H, s), 9.15 (2H, t, J=1.8 Hz), 8.57 (1H, dd, J=1.3, 8.2 Hz), 7.96-7.94 (1H, m) ppm.

### Description 40

### 3-[3-Trifluoromethylpyridin-2-yl]imidazo[1,2-b][1,2,4]triazin-7-amine

Description 39 (285 mg, 0.92 mmol) was dissolved in ethanol (15 ml) and ethyl acetate (15 ml) and Lindlar's catalyst (260 mg) added. The reaction was stirred under an atmosphere of hydrogen for 24 h, then filtered through celite (washing with ethyl acetate), preadsorbed onto silica gel and purified by column chromatography (60% ethyl acetate/iso-hexane) to give the title compound (110 mg, 43%) as a red solid. MS: (ES (M+1)) 281. ¹H NMR (400 MHz, DMSO) δ 8.99 (2H, t, J=5.4 Hz), 8.41 (1H, dd, J=1.4, 8.1 Hz), 7.74 (1H, dd, J=4.7, 7.3 Hz), 7.51 (1H, s), 6.20 (2H, s) ppm.

### Description 41

### 2-Amino-4-(3-trifluoromethyl-2-pyridyl)pyridine

Description 7 (1.5 g, 5.28 mmol) and hydrazine hydrate (1.28 g, 25.6 mmol) were suspended in isopropyl alcohol (30 ml), and the resulting mixture was heated to reflux overnight. The volatiles were removed under reduced pressure, and the residue was azeotroped with toluene. The resulting pale yellow solid was dissolved in ethanol (100 ml) and hydrogenated over Raney nickel (aqueous suspension, 4 ml) at atmospheric pressure (H₂ balloon) for 72 h. The mixture was filtered through Celite®, and the filtrate was hydrogenated over 10% Pd/C on a Parr® at 60 psi for 16 h. The mixture was filtered through Celite® and the filtrate was concentrated. The residue was adsorbed onto Celite® and purified by flash chromatography (Biotage 40S®) eluting with DCM/MeOH/ammonium hydroxide 97.5/2.5/0.15 to 95/5/0.3 to give a colourless oil (367 mg, 30%). MS: (ES (M+1)) 240; ¹H NMR δ (ppm)(CDCl₃): 4.52 (2H, s), 6.61 (1H, s), 6.78 (1H, d, J=5.2 Hz), 7.47 (1H, dd, J=5.0, 8.1 Hz), 8.09 (1H, d, J=8.1 Hz), 8.17 (1H, d, J=5.2 Hz), 8.85 (1H, d, J=4.2 Hz).

### Description 42

### 7-[3-trifluoromethylpyridin-2-yl]imidazo[1,2-a]pyridine

Prepared from Description 41, using a procedure similar to Description 12. MS: (ES (M+1)) 264; 1H NMR (ppm)(CDCl₃): 7.03 (1H, dd, J=1.6, 7.0 Hz), 7.47-7.49 (1H, m), 7.67 (1H, br s), 7.74 (1H, d, J=1.1 Hz), 7.83 (1H, br s), 8.13 (1H, dd, J=1.6, 8.1 Hz), 8.22 (1H, dd, J=0.8, 7.0 Hz), 8.87 (1H, d, J=7.0 Hz).

### Description 43

### 3-Nitro-7-[3-trifluoromethylpyridin-2-yl]imidazo[1,2-a]-pyridine

Description 42 (400 mg, 1.52 mmol) was dissolved in concentrated sulfuric acid (8 ml), and the resulting mixture was stirred at 0° C., while 1:1 c.H₂SO₄/70% HNO₃ (0.2 ml) was added, and then warmed to room temperature, with stirring for 2 h. A further 0.2 ml of the above nitric acid solution was added, and stirring was continued for a further 2 h. The mixture was poured onto ice, neutralised with ammonium hydroxide solution, and extracted with EtOAc (×3). The combined organic phases were washed (brine), dried (sodium sulfate) and concentrated to give a pale brown solid (468 mg, 99%). MS: (ES (M+1)) 309; ¹H NMR δ (ppm)(CDCl₃): 7.50 (1H, d, J=7.2 Hz), 7.58 (1H, dd, J=4.1, 7.6 Hz), 8.03 (1H, s), 8.19 (1H, d, J=8.0 Hz), 8.70 (1H, s), 8.93 (1H, d, J=4.1 Hz), 9.48 (1H, d, J=7.2 Hz).

### Description 44

### 2-Methylthio-4-[3-trifluoromethylpyridin-2-yl]pyrimidine

A mixture of Description 26 (3.27 g, 17.3 mmol) and N,N-dimethylformamide dimethyl acetal (4.6 ml, 34.6 mmol) was heated at 100° C. for 2 h. The excess N,N-dimethylformamide dimethyl acetal was removed in vacuo to give the crude adduct (4.1 g). A portion (1.06 g, 4.34 mmol) was added to ethanol (10 ml) then thiourea (660 mg, 8.68 mmol) and 1N ethanolic KOH (4.4 ml, 4.4 mmol) were added. The mixture was heated at reflux for 4 h. Additional 1N ethanolic KOH (4.4 ml, 4.4 mmol) was added and the mixture heated at reflux for a further 1 h. After cooling, flash silica (ca. 50 ml) was added and the solvent evaporated. Purification by flash column chromatography (eluant 5% MeOH—CH₂Cl₂ containing 1% AcOH) gave 4-[3-trifluoromethylpyridin-2-yl]pyrimidine-2-thiol (1.39 g) (MS: (ES (M+1)) 258. To this product, acetonitrile (15 ml) and potassium carbonate (2.5 g, 18 mmol) were added. Iodomethane (300 μl, 4.77 mmol) was then added and the reaction stirred for 20 min. The acetonitrile was evaporated and water (30 ml) added and the mixture then extracted with ethyl acetate (2×30 ml). The combined organic layers were evaporated and the residue purified by flash column chromatography (eluant 25% EtOAc/isohexane, then 40% EtOAc/isohexane) to give the title compound as a white crystalline solid (580 mg).

¹H NMR (400 MHz, CDCl₃) δ 8.87 (1H, dd, J=1.4, 4.7 Hz), 8.67 (1H, d, J=5.0 Hz), 8.16 (1H, dd, J=1.4, 8.3 Hz), 7.56-7.52 (1H, m), 7.40 (1H, d, J=5.0 Hz), 2.59 (3H, s).

### Description 45

### 4-[3-Trifluoromethylpyridin-2-yl]pyrimidine-2-carbonitrile

3-Chloroperoxybenzoic acid (77%, 420 mg, 2.43 mmol) was added to a solution of Description 44 (300 mg, 1.1 mmol) in dichloromethane (10 ml) at room temperature. The reaction was stirred for 3 h, then diluted with dichloromethane (60 ml) and the solution washed with 5% aq. NaHSO₃ solution (40 ml) then 10% aq. K₂CO₃ solution (40 ml). The organic layer was dried (Na₂SO₄) and evaporated to give 2-methylsulfonyl-4-[3-trifluoromethylpyridin-2-yl]pyrimidine (318 mg, 1.05 mmol). This product was dissolved in N,N-dimethylformamide (10 ml) and sodium cyanide (130 mg, 2.6 mmol) was added. The mixture was heated to 100° C. for 10 min, then cooled to room temperature. Water (30 ml) was added and the mixture extracted with ethyl acetate (2×30 ml). The combined organic layers were washed with water (2×30 ml), brine (10 ml), then dried (Na₂SO₄) and evaporated to give the title compound (243 mg). ¹H NMR (400 MHz, CDCl₃) δ 9.01 (1H, d, J=5.1 Hz), 8.91 (1H, dd, J=1.3, 4.9 Hz), 8.22 (1H, dd, J=1.3, 8.2 Hz), 8.00 (1H, d, J=5.1 Hz), 7.64-7.62 (1H, m).

### Description 46

### 5-Chloro-2-(tetrahydro-2H-pyran-2-yl)pyridazin-3(2H)-one

A mixture of 5-chloropyridazin-3-one (13.14 g, 100 mmol), 3,4-dihydro-2H-pyran (13 ml, 142 mmol) and para-toluenesulfonic acid (1.6 g, 8.4 mmol) in anhydrous tetrahydrofuran (150 ml) was heated at 80° C. for 36 h. After cooling to room temperature and addition of 50 ml saturated aqueous sodium carbonate solution the mature was concentrated under reduced pressure and repartitioned between ethyl acetate/water (500/100 ml). The phases were separated and the aqueous phase was extracted two times with ethyl acetate (200 ml each). The combined organic layers were washed with brine, dried over sodium sulfate and adsorbed onto silica gel in the presence of triethylamine. Purification by flash chromatography (50% ethyl acetate in hexane) gave the title compound as a light brown solid (12 g, 59%), MS: (ES (M+1)) 203.

¹H NMR (360 MHz, CDCl₃) δ 1.50-2.18 (6H, m), 3.71-3.78 (1H, m), 4.08-4.15 (1H, m), 6.00 (1H, dd, J-10.5 and 2), 6.96 (1H, d, J=2.4), 7.79 (1H, d, J=2.4) ppm.

### Final Products

### EXAMPLE 1

### N-(4-Trifluoromethylphenyl)-7-(3-trifluoromethyl-2-pyridyl)[1,2,4]triazolo[4,3-b]pyridazine-3-amine

To a mixture of Description 2 (3.5 g, 13.8 mmol) in anhydrous isopropanol (20 ml) was added hydrazine monohydrate (3,4 ml, 70 mmol) and the mixture was heated at 100° C. for 15 h. After cooling to room temperature the solution was concentrated under reduced pressure and toluene was added to the resulting oil. The mixture was concentrated under reduced pressure again and the whole procedure was repeated twice to yield 3-hydrazino-5-(3-trifluoromethyl-2-pyridyl)pyridazine (3.2 g, 91%) as a red syrup which crystallises over 3 days at room temperature.

The pyridazine (0.56 g, 2.2 mmol) was dissolved in dry acetonitrile (10 ml) and a solution of 4-trifluoromethylphenylisocyanate (0.43 g, 2.3 mmol) in 3 ml acetonitrile was added dropwise while stirring at room temperature. The solution was heated at 90° C. for 12 h and cooled to room temperature. Phosphorous oxychloride (0.41 ml, 4.4 mmol) was added dropwise to the suspension and the resulting mixture was heated under reflux for 12 h. After addition of more phosphorous oxychloride (0.41 ml, 4.4 mmol) the mixture was heated for another 12 h under reflux and tlc showed complete conversion of starting material. The resulting yellow solution was poured onto a mixture of chloroform and water (200/20 ml) and the pH was adjusted to 8 by portionwise addition of saturated aqueous sodium carbonate solution and the phases were separated. After two further extractions the combined organic extracts were washed with water and brine and dried over sodium sulfate. After filtration the compound was adsorbed onto silica gel and purified by flash column (50% ethyl acetate) to yield the title compound (0.45 g, 48%) as a canary-yellow solid, MS: (ES (M+1)) 425.

¹H NMR (360 MHz, DMSO) δ 7.70 (2H, d, J=8.7), 7.81 (1H, dd, J=8.0 and 4.6), 8.07 (2H, d, J=8.7), 8.37 (1H, d, J=1.4), 8.45 (1H, d, J=8.0), 8.77 (1H, d, J=1.4), 9.03 (1H, d, J=4.6), 10.32 (1H, s) ppm.

### EXAMPLE 2

### N-(4-tert-Butyl)phenyl)-7-(3-trifluoromethyl-2-pyridyl)[1,2,4]triazolo[4,3-b]pyridazine-3-amine

Using a procedure analogous to Example 1 but starting with N-(4-tert-butyl)phenylisocyanate the title compound was obtained (0.16 g, 55%) as a bright yellow solid, MS: (ES (M+1)) 413.

¹H NMR (400 MHz, DMSO) δ 1.29 (9H, s), 7.36 (211, d, J=8.7), 7.77-7.79 (3H, m), 8.31 (1H, d, J=1.6), 8.43 (1H, d, J=8.0), 8.70 (1H, d, J=1.6), 9.03 (1H, d, J=4.8), 9.65 (1H, s) ppm.

### EXAMPLE 3

### N-phenyl-7-[3-trifluoromethyl-2-pyridinyl][1,2,4]triazolo[4,3-b]pyridazin-3-amine

Using a procedure analogous to that given in Example 1 except phenylisocyanate was used as a starting material and after the aqueous work-up the crude material obtained was dissolved in DMSO and purified using preparative LC-MS eluting with a pH 10 eluent.

¹H NMR (400 MHz, DMSO); 6.97 (1H, t, J=7.2 Hz), 7.35 (2H, t, J=7.8 Hz), 7.81 (1H, dd, J=4.8, 8.0 Hz), 7.87 (2H, d, J=8.0 Hz), 8.32 (1H, d, J=1.2 Hz), 8.44 (1H, d, J=8.0 Hz), 8.71 (1H, d, J=1.6 Hz), 9.03 (1H, d, J=4.4 Hz), 9.76 (1H, s).

Examples 4-30 were similarly prepared according to the procedures described above:

### EXAMPLE 4

### N-[2-trifluoromethylphenyl]-7-[3-trifluoromethyl-2-pyridinyl][1,2,4]triazolo[4,3-b]pyridazin-3-amine

¹H NMR (400 MHz, DMSO); 7.25-7.27 (1H, m), 7.64-7.67 (1H, m), 7.74 (2H, d, J=7.6 Hz), 7.79-7.87 (2H, m), 8.39 (1H, s), 8.44 (1H, d, J=8.0 Hz), 8.75 (1 H, s), 9.02 (1H, d, J=4.8 Hz)

### EXAMPLE 5

### N-(3-chlorophenyl)-7-[3-trifluoromethyl-2-pyridinyl][1,2,4]triazolo[4,3-b]Pyridazin-3-amine

¹H N (400 MHz, DMSO); 7.01-7.03 (1H, d, J=7.6 Hz), 7.37 (1H, t, J=8.2 Hz), 7.78-7.82 (2H, m), 8.07 (1H, s), 8.34 (1H, 9), 8.44 (1H, d, J=7.6 Hz), 8.73 (1H, s), 9.02 (1H, d, J=4.4 Hz), 10.09 (1H, s)

### EXAMPLE 6

### N-[3-trifluoromethylphenyl]-7-[3-trifluoromethyl-2-pyridinyl][1,2,4]triazolo[4,3-b]pyridazin-3-amine

¹H NMR (400 MHz, DMSO); 7.31 (1H, d, J=7.6 Hz), 7.59 (1H, t, J=8.0 Hz), 7.80 (1H, dd, J=4.8, 7.6 Hz), 8.11 (1H, d, J=8.0 Hz), 8.36 (2H, d, J=3.6 Hz), 8.44 (1H, d, J=8.0 Hz), 8.74 (1H, d, J=1.6 Hz), 9.02 (1H, d, J=4.4 Hz), 10.25 (1H, s)

### EXAMPLE 7

### N-(2,4-difluorophenyl)-7-[3-trifluoromethyl-2-pyridinyl][1,2,4]triazolo[4,3-b]pyridazin-3-amine

¹H NMR (400 MHz, DMSO); 7.12 (1H, m), 7.35 (1H, m)), 7.80-7.90 (2H, m), 8.32 (1H, s), 8.43 (1H, d, J=7.4 Hz), 8.70 (1H, s), 9.01 (1H, d, J=1.6 Hz)

### EXAMPLE 8

### N-[4-methoxyphenyl]-7-[3-trifluoromethyl-2-pyridinyl][1,2,4]triazolo[4,3-b]pyridazin-3-amine

¹H NMR (400 MHz, DMSO); 3.74 (3H, s), 6.94 (2H, d, J=8.8 Hz), 7.79 (3H, m), 8.26 (1H, s), 8.43 (1H, d, J=8.4 Hz), 8.67 (1H, d, J=1.6 Hz), 9.01 (1H, d, J=4.8 Hz), 9.55 (1H, s)

### EXAMPLE 9

### N-[2-(1-methylethyl)phenyl]-7-[3-trifluoromethyl-2-pyridinyl][1,2,4]triazolo[4,3-b]pyridazin-3-amine

¹H NMR (400 MHz, DMSO); 1.21 (6H, d, J=7.2 Hz), 3.31 (1H, septet), 7.14-7.19 (2H, m), 7.35 (1H, d, J=7.6 Hz), 7.50 (1H, d, J=7.6 Hz), 7.79 (1H, dd, J=5.2, 8.0 Hz), 8.27 (1H, s), 8.43 (1H, d, J=8.0 Hz), 8.53 (1H, s), 8.67 (1H, d, J=1.2 Hz), 9.01 (1H, d, J=4.8 Hz)

### EXAMPLE 10

### N-[3-methylsulfanylphenyl]-7-[3-trifluoromethyl-2-pyridinyl][1,2,4]triazolo[4,3-b]pyridazin-3-amine

¹H NMR (400 MHz, DMSO); 2.54 (3H, s), 6.87 (1H, d, J=7.6 Hz), 7.29 (1H, t, J=8.0 Hz), 7.66 (1H, d, J=7.6 Hz), 7.81 (1H, m), 7.87 (1H, s), 8.33 (1H, s), 8.45 (1H, d, J=8.0 Hz), 8.72 (1H, s), 9.03 (1H, d, J=4.4 Hz), 9.83 (1H, s)

### EXAMPLE 11

### N-(2-naphthalenyl)-7-[3-trifluoromethyl-2-pyridinyl][1,2,4]triazolo[4,3-b]pyridazin-3-amine

¹H NMR (400 MHz, DMSO); 7.37 (1H, m), 7.46 (1H, m), 7.80-7.92 (5H, m), 8.37 (1H, s), 8.45 (1H, d, J=8.0 Hz), 8.54 (1H, s), 8.75 (1H, d, J=1.2 Hz), 9.04 (1H, d, J=4.4 Hz), 10.04 (1H, s)

### EXAMPLE 12

### N-{4-trifluoromethoxyphenyl}-7-[3-trifluoromethyl-2-pyridinyl][1,2,4]triazolo[4,3-b]pyridazin-3-amine

¹H NMR (400 MHz, DMSO); 7.36 (2H, d, J=8.8 Hz), 7.81 (1H, dd, J=4.8, 8.0 Hz), 7.98 (2H, d, J=9.2 Hz), 8.33 (1H, d, J=0.4 Hz), 8.44 (1H, d, J=8.0 Hz), 8.73 (1H, d, J=1.6 Hz), 9.02 (1H, d, J=4.4 Hz), 10.06 (1H, s)

### EXAMPLE 13

### N-(2-phenylethyl)-7-[3-trifluoromethyl-pyridinyl][1,2,4]triazolo[4,8-b]pyridazin-3-amine

¹H NMR (400 MHz, DMSO); 3.00 (2H, t, J=7.4 Hz), 3.67 (2H, m), 5.88 (1H, t, J=7.4 Hz), 7.09 (1H, t, J=5.6 Hz), 7.19 (1H, m), 7.27-7.32 (4H, m), 7.77 (1H, dd, J=4.8, 7.6 Hz), 8.13 (1H, s), 8.41 (1H, d, J=8.0 Hz), 8.53 (1H, d, J=1.6 Hz), 8.99 (1H, d, J=4.4 Hz)

### EXAMPLE 14

### N-(1,3-benzodioxol-5-yl)-7-[3-trifluoromethyl-2-pyridinyl][1,2,4]triazolo[4,3-b]pyridazin-3-amine

¹H NMR (400 MHz, DMSO); 5.99 (2H, s), 6.90 (1H, d, J=8.8 Hz), 7.37 (1H, dd, J=1.6, 8.0 Hz), 7.55 (1H, d, J=1.2 Hz), 7.80 (1H, dd, J=5.2, 7.2 Hz), 8.29 (1H, s), 8.43 (1H, m), 8.68 (1H, s), 9.02 (1H, d, J=4.8 Hz), 9.66 (1H, s)

### EXAMPLE 15

### N-[3-fluorophenylmethyl]-7-[3-trifluoromethyl-2-pyridinyl][1,2,4]triazolo[4,3-b]pyridazin-3-amine

¹H NMR (400 MHz, DMSO); 4.66 (2H, d, J=6.4 Hz), 7.07 (1H, m), 7.26 (1H, m), 7.37 (1H, m), 7.72-7.80 (2H, m), 8.15 (1H, d, J=1.2 Hz), 8.42 (1H, d, J=8.0 Hz), 8.58 (1H, d, J=2.0 Hz), 9.00 (1H, d, J=4.8 Hz)

### EXAMPLE 16

### 2-({7-[3-trifluoromethyl-2-pyridinyl][1,2,4]triazolo[4,3-b]pyridazin-3-yl}amino)benzonitrile

¹H NMR (400 MHz, DMSO); 7.52 (1H, t, J=7.4 Hz), 7.81-7.93 (3H, m), 8.35 (1H, dd, J=0.8, 8.0 Hz), 8.40 (1H, d, J=1.6 Hz), 8.48 (1H, d, J=8.0 Hz), 8.77 (1H, d, J=1.6 Hz), 9.06 (1H, d, J=4.4 Hz)

### EXAMPLE 17

### N-(diphenylmethyl)-7-[3-trifluoromethyl-2-pyridinyl][1,2,4]triazolo[4,3-b]pyridazin-3-amine

¹H NMR (400 MHz, DMSO); 6.29 (1H, d, J=9.2 Hz), 7.25 (2H, m), 7.34 (4H, m), 7.51 (4H, m)), 7.75 (1H, m), 7.95 (1H, d, J=9.2 Hz), 8.15 (1H, s), 8.41 (1H, d, J=8.4 Hz), 8.59 (1H, d, J=1.6 Hz), 8.99 (1H, s)

### EXAMPLE 18

### N-[(1S)-1-phenylethyl]-7-[3-trifluoromethyl-2-pyridinyl][1,2,4]triazolo[4,3-b]pyridazin-3-amine

¹H NMR (400 MHz, DMSO); 1.59 (3H, d, J=6.8 Hz), 5.09 (1H, m), 7.21 (1H, m), 7.31 (2H, t, J=7.6 Hz), 7.48 (2H, d, J=7.2 Hz), 7.55 (1H, d, J=8.4 Hz), 7.77 (1H, dd, J=4.8, 8.0 Hz), 8.11 (1H, d, J=1.2 Hz), 8.41 (1H, d, J=7.6 Hz), 8.56 (1H, d, J=2.0 Hz), 8.99 (1H, d, J=4.4 Hz)

### EXAMPLE 19

### N-(2,4-dichlorophenyl)-7-[3-trifluoromethyl-2-pyridinyl][1,2,4]triazolo[4,3-b]pyridazin-3-amine

¹H NMR (400 MHz, DMSO); 7.44 (1H, dd, J=2.4, 8.8 Hz), 7.69 (1H, d, J=2.4 Hz), 7.80 (1H, dd, J=4.8, 7.6 Hz), 7.98 (1H, d, J=8.8 Hz), 8.38 (1H, d, J=0.8 Hz), 8.44 (1H, d, J=8.0 Hz), 8.75 (1H, d, J=1.6 Hz), 9.02 (1H, d, J=4.4 Hz)

### EXAMPLE 20

### N-(3,4-dichlorophenyl)-7-[3-trifluoromethyl-2-pyridinyl][1,2,4]triazolo[4,3-b]pyridazin-3-amine

¹H NMR (400 MHz, DMSO); 7.60 (1H, d, J=8.8 Hz), 7.79-7.87 (2H, m), 8.27 (1H, d, J=2.4 Hz), 8.36 (1H, s), 8.45 (1H, d, J=8.0 Hz), 8.75 (1H, d, J=1.2 Hz), 9.03 (1H, d, J=4.8 Hz), 10.27 (1H, s)

### EXAMPLE 21

### N-[4-dimethylaminophenyl]-N-{7-[3-trifluoromethyl-2-pyridinyl][1,2,4]triazolo[4,3-b]pyridazin-3-yl}amine

¹H NMR (400 MHz, DMSO); 2.82 (6H, s), 6.68 (2H, d, J=8.8 Hz), 7.23 (2H, d, J=8.8 Hz), 7.80 (1H, dd, J=5.2, 7.6 Hz), 8.13 (1H, s), 8.43 (1H, d, J=8.0 Hz), 8.65 (1H, s), 9.02 (1H, d, J=4.8 Hz), 9.34 (1H, s)

### EXAMPLE 22

### N-[(3,4-dichlorophenyl)methyl]-7-[3-trifluoromethyl-2-pyridinyl][1,2,4]triazolo[4,3-b]pyridazin-3-amine

¹H NMR (400 MHz, DMSO); 4.63 (2H, d, J=6.4 Hz), 7.43 (1H, dd, J=1.6, 8.4 Hz), 7.59 (1H, d, J=8.4 Hz), 7.70 (1H, s), 7.76-7.79 (2H, m), 8.15 (1H, s), 8.42 (1H, d, J=8.0 Hz), 8.58 (1H, d, J=1.6 Hz), 9.00 (1H, d, J=4.8 Hz)

### EXAMPLE 23

### N-(4-chloro-2-methylphenyl)-7-[3-trifluoromethyl-2-pyridinyl][1,2,4]triazolo[4,3-b]pyridazin-3-amine

¹H NMR (400 MHz, DMSO); 2.33 (3H, s), 7.25 (1H, dd, J=2.4, 8.8 Hz), 7.33 (1H, d, J=2.0 Hz), 7.67 (1H, d, J=8.8 Hz), 7.80 (1H, dd, J=4.8, 8.0 Hz), 8.32 (1H, d, J=1.6 Hz), 8.44 (1H, d, J=8.0 Hz), 8.58 (1H, s), 8.70 (1H, d, J=1.6 Hz), 9.02 (1H, d, J=4.4 Hz)

### EXAMPLE 24

### N-(3-chloro-4-fluorophenyl)-7-[3-trifluoromethyl-2-pyridinyl][1,2,4]triazolo[4,3-b]pyridazin-3-amine

¹H NMR (400 MHz, DMSO); 7.41 (1H, t, J=9.0 Hz), 7.79-7.84 (2H, m), 8.19 (1H, dd, J=2.4, 6.4 Hz), 8.33 (1H, d, J=1.6 Hz), 8.44 (1H, d, J=8.0 Hz), 8.73 (1H, d, J=1.6 Hz), 9.02 (1H, d, J=4.4 Hz), 10.11 (1H, s)

### EXAMPLE 25

### N-[2-fluoro-6-trifluoromethylphenyl]-7-[3-trifluoromethyl-2-pyridinyl][1,2,4]triazolo[4,3-b]pyridazin-3-amine

¹H NMR (400 MHz, DMSO); 7.59 (1H, m), 7.69 (3H, m), 7.80 (1H, dd, J=4.8, 8.0 Hz), 8.27 (1H, s), 8.44 (1H, d, J=8.0 Hz), 8.67 (1H, s), 9.02 (1H, d, J=4.8 Hz)

### EXAMPLE 26

### N-[4-fluoro-2-trifluoromethylphenyl]-7-[3-trifluoromethyl-2-pyridinyl][1,2,4]triazolo[4,3-b]pyridazin-3-amine

¹H NMR (400 MHz, DMSO); 7.57 (1H, m), 7.66 (1H, dd, J=2.4, 8.8 Hz), 7.80 (1H, dd, J=4.8, 8.0 Hz), 7.88 (1H, dd, J=4.8, 8.8 Hz), 8.35 (1H, s), 8.44 (1H, d, J=8.0 Hz), 8.72 (1H, s), 9.02 (1H, d, J=4.8 Hz)

### EXAMPLE 27

### N-[4-fluoro-3-trifluoromethylphenyl]-7-[3-trifluoromethyl-2-pyridinyl][1,2,4]triazolo[4,3-b]pyridazin-3-amine

¹H NMR (400 MHz, DMSO); 7.52 (1H, t, J=9.8 Hz), 7.81 (1H, dd, J=4.8, 7.6 Hz), 8.18 (1H, m), 8.34 (1H, d, J=1.2 Hz), 8.39-8.45 (2H, m), 8.75 (1H, d, J=1.6 Hz), 9.02 (1H, d, J=4.4 Hz)

### EXAMPLE 28

### N-[2-chloro-4-trifluoromethylphenyl]-7-[3-trifluoromethyl-2-pyridinyl][1,2,4]triazolo[4,3-b]pyridazin-3-amine

¹H NMR (400 MHz, DMSO); 7.69 (2H, m), 7.81 (1H, dd, J=4.8, 8.0 Hz), 7.93 (1H, s), 8.02 (1H, d, J=8.4 Hz), 8.45 (2H, d, J=6.4 Hz), 8.80 (1H, d, J=1.6 Hz), 9.02 (1H, d, J=4.4 Hz)

### EXAMPLE 29

### N-(2,3-dihydro-1H-inden-5-yl)-7-[3-trifluoromethyl-2-pyridinyl][1,2,4]triazolo[4,3-b]pyridazin-3-amine

¹H NMR (400 MHz, DMSO); 1.99-2.06 (2H, m), 2.80-2.89 (4H, m), 7.17 (1H, d, J=8.4 Hz), 7.57 (1H, d, J=8.0 Hz), 7.80 (2H, d, J=8.0 Hz), 8.28 (1H, s), 8.43 (1H, d, J=8.0 Hz), 8.68 (1H, s), 9.02 (1H, d, J=4.4 Hz), 9.58 (1H, 9)

### EXAMPLE 30

### N-(2,3-dihydro-1,4-benzodioxin-6-yl)-7-[3-trifluoromethyl-2-pyridinyl][1,2,4]triazolo[4,3-b]pyridazin-3-amine

¹H NMR (400 MHz, DMSO); 4.16-4.24 (4H, m), 6.74 (1H, 9), 6.83 (1H, d, J=8.8 Hz), 7.30 (1H, dd, J=2.4, 8.8 Hz), 7.48 (1H, d, J=2.4 Hz), 7.79 (1H, dd, J=4.8, 7.2 Hz), 8.26 (1H, s), 8.43 (1H, d, J=8.0 Hz), 9.01 (1H, d, J=4.4 Hz), 9.56 (1H, s)

### EXAMPLE 31

### N-(4-Trifluoromethylphenyl)-7-(3-methyl-2-pyridyl)-[1,2,4]triazolo[4,3-b]pyridazine-3-amine

To a mixture of Description 1 (0.38 g, 1.85 mmol) in anhydrous isopropanol (10 ml) was added hydrazine monohydrate (0.5 ml, 10.3 mmol) and the mixture was heated at 100° C. for 15 h. After cooling to room temperature the solution was concentrated under reduced pressure and toluene was added to the resulting oil. The mixture was concentrated under reduced pressure again and the whole procedure was repeated twice to yield 3-hydrazino-5-(3-methyl-2-pyridyl)pyridazine (0.36 g, 95%) as a red syrup.

The pyridazine (0.36 g, 1.8 mmol) was suspended in a mixture of dry p-Xylene/N,N-dimethylacetamide (10 ml each) and 4-trifluoromethylphenyl isothiocyanate (0.38 g, 1.87 mmol) was added in one portion while stirring at room temperature. The mixture was heated at 100° C. for 1 h and cooled to room temperature. Dicyclohexylcarbodiimide (0.39 g, 1.89 mmol) was added in one portion and the resulting mixture was heated at 100° C. for 1 h, poured onto a mixture of chloroform and water (200/20 ml) and the phases were separated. After two further extractions the combined organic extracts were washed with water and brine and dried over sodium sulfate. After filtration the compound was adsorbed onto silica gel and purified by flash column (ethyl acetate) to yield the title compound (0.11 g, 16%) as a yellow solid, MS: (ES (M+1)) 370.

¹H NMR (360 MHz, DMSO) δ 2.52 (3H, s), 7.44 (1H, dd, J=7.5 and 4.5 Hz), 7.69 (2H, d, J=8.7 Hz), 7.84 (1H, d, J=7.5 Hz), 8.07 (2H, d, J=8.7 Hz), 8.46 (1H, d, J=1.7 Hz), 8.60 (1H, d, J=4.5 Hz), 8.82 (1H, d, J=1.7 Hz), 10.28 (1H, s) ppm.

### EXAMPLE 32

### 5-Chloro-7-(3-methyl-2-pyridyl)-N-(4-trifluoromethylphenyl)-[1,2,4]triazolo[4,3-a]pyridin-3-amine

To a mixture of Description 6 (0.75 g, 3.14 mmol) in anhydrous isopropanol (20 ml) was added hydrazine monohydrate (0.6 ml, 12.3 mmol) and the mixture was heated at 100° C. for 15 h. After cooling to room temperature the solution was concentrated under reduced pressure and water (20 ml) was added to the resulting oil. The aqueous layer was decanted, toluene (20 ml) was added to the resulting wet solid and the solvent was removed under reduced pressure. The procedure was repeated twice to yield 6-hydrazino-2-chloro-4-(3-methyl-2-pyridyl)pyridine (0.7 g, 95%) as a brown solid.

The pyridine (0.7 g, 3 mmol) was suspended in a mixture of dry p-xylene/N,N-dimethylacetamide (10 ml each) and 4-trifluoromethylphenylisothiocyanate (0.61 g, 3 mmol) was added in one portion while stirring at room temperature. The mixture was heated at 60° C. for 1 h and cooled to room temperature. Dicyclohexylcarbodiimide (0.62 g, 3 mmol) was added in one portion and the resulting mixture was heated at 100° C. for 1 h, poured onto a mixture of chloroform and water (200/20 ml) and the phases were separated. After two further extractions the combined organic extracts were washed with water and brine and dried over sodium sulfate. After filtration the compound was adsorbed onto silica gel and purified by flash column (ethyl acetate) to yield the title compound (0.48 g, 40%) as a greenish yellow solid, MS: (ES (M+1)) 404/406.

¹H NMR (360 MHz, DMSO) δ 2.50 (3H, s), 7.02 (2H, d, J=8.5 Hz), 7.32 (1H, d, J=1.2 Hz), 7.40 (1H, dd, J=7.5 and 4.7 Hz), 7.56 (2H, d, J=8.5 Hz), 7.81 (1H, d, J=7.5 Hz), 7.96 (1H, d, J=1.2 Hz), 8.55 (1H, d, J=4.7 Hz), 9.17 (1H, s) ppm.

### EXAMPLE 33

### 5-Chloro-7-(2-methoxyphenyl)-N-(4-trifluoromethylphenyl)[1,2,4]triazolo[4,3-a]pyridin-3-amine

Using a procedure analogous to that given in Example 32 and the precursor intermediate in Description 8:

(0.16 g, 55%) as an off white solid, MS: (ES (M+1)) 419/421.

¹H NMR (360 MHz, DMSO) δ 3.85 (3H, s), 6.95 (2H, d, J=8.5 Hz), 7.08 (1H, m), 7.19 (1H, d, J=7.5 Hz), 7.28 (1H, d, J=1.2 Hz), 7.42-7.55 (4H, m), 7.83 (1H, d, J=1.2 Hz), 9.12 (1H, s) ppm.

### EXAMPLE 34

### 5-Chloro-N-(4-trifluoromethylphenyl)-7-(3-trifluoromethyl-2-pyridyl)[1,2,4]triazolo[4,3-a]pyridin-3-amine

Using a procedure analogous to that given in Example 32 and the precursor intermediate in Description 7:

(0.39 g, 68%) of an off-white solid, MS: (ES (M+1)) 458/460.

¹H NMR (360 MHz, CDCl₃) δ 7.03 (1H, s br.), 7.47 (2H, d, J=8.6 Hz), 7.55 (1H, d, J=8.0 and 4.9 Hz), 7.59 (2H, d, J=8.6 Hz), 7.77 (1H, s br.), 8.16 (1H, d, J=8.0 Hz), 8.89 (1H, d, J=4.9 Hz) ppm.

### EXAMPLE 35

### 6-Chloro-N-(5-isoquinolyl)-7-(4-trifluoromethylphenyl)[1,2,4]triazolo[4,3-b]pyridazin-3-amine

Using a procedure analogous to that given in Example 32 but employing 5-isoquinolylisocyanate and the intermediate in Description 5:

(0.16 g, 55%) as a bright yellow solid, MS: (ES (M+1)) 441/443.

¹H NMR (360 MHz, DMSO) δ 7.63-7.67 (1H, m), 7.82-7.87 (4H, m), 7.93 (2H, d, J=8.0 Hz), 8.09 (1H, d, J=6.0 Hz), 8.47 (1H, s), 8.54 (1H, d, J=6.0 Hz), 9.34 (1H, s), 9.74 (1H, s) ppm.

### EXAMPLE 36

### 7-(3-methyl-2-pyridyl)-N-(4-trifluoromethylphenyl) [1-2,4]triazolo[4,3-a]pyridin-3-amine

To a mixture of Example 32 (0.15 g, 0.38 mmol) in anhydrous ethanol (10 ml) was added ammonium formate (100 mg, 1.6 mmol) and palladium on carbon (1 spatula) and the mixture was heated at 80° C. for 15 h. After cooling to room temperature the solution was filtered through highflow, poured onto water (20 ml) and the resulting solid was filtered, washed and dried on the sinter. Recrystallisation from acetonitrile yielded the title compound (0.11 g, 78%) as a colourless solid, MS: (ES (M+1)) 369.

¹H NMR (360 MHz, DMSO) δ 2.47 (3H, s), 7.21 (1H, d br., J=7.2 Hz), 7.38 (1H, dd, J=7.7 and 4.7 Hz), 7.68 (2H, d, J=8.7 Hz), 7.77-7.84 (4H, m), 8.46 (1H, d br., J=7.2 Hz), 8.54 (1H, d, J=4.7 Hz), 9.83 (1H, s) ppm.

### EXAMPLE 37

### 7-(3-Trifluoromethyl-2-pyridyl)-N-(4-trifluoromethylphenyl)[1,2,4]triazolo[4,3-a]pyridin-3-amine

Using a procedure analogous to that given in Example 36 and employing the compound described in Example 34:

(0.14 g, 42%) of a colourless solid, MS: (ES (M+1)) 424.

¹H NMR (360 MHz, DMSO) δ 6.33 (1H, d, J=7.2 Hz), 6.84 (4H, s), 6.93 (1H, dd, J=7.2 and 4.6 Hz), 6.95 (1H, s br.), 7.57 (2H, d, J=7.3 Hz), 8.13 (1H, d, J=4.6 Hz), 9.51 (1H, s) ppm.

### EXAMPLE 38

### 7-(2-Methoxyphenyl)-N-(4-trifluoromethylphenyl)[1,2,4]triazolo[4,3-a]pyridin-3-amine

Using a procedure analogous to that given in Example 36 and employing the compound described in Example 33:

(0.14 g, 42%) of a colourless solid, MS: (ES (M+1)) 385.

¹H NMR (500 MHz, DMSO) δ 3.87 (3H, s), 7.11-7.15 (1H, m), 7.22 (1H, d, J=8.0 Hz), 7.48-7.51 (1H, m), 7.57 (1H, d, J=7.0 Hz), 7.69 (2H, d, J=7.9 Hz), 8.05 (2H, d, J=7.9 Hz), 8.26 (1H, d, J=1.2 Hz), 8.75 (1H, d, J=1.2 Hz), 10.19 (1H, s) ppm.

### EXAMPLE 39

### N-(5-isoquinolyl)-7-(4-trifluoromethylphenyl)[1,2,4]triazolo[4,3-b]pyridazine-3-amine

Using a procedure analogous to that given in Example 36 and employing the compound described in Example 35:

(0.14 g, 42%) of a green solid, MS: (ES (M+1)) 407.

¹H NMR (360 MHz, DMSO) δ 7.59-7.64 (1H, m), 7.77-7.79 (1H, m, br.), 7.87-7.94 (3H, m), 8.06 (2H, d, J=8.0 Hz), 8.13-8.16 (1H, m, br.), 8.56-8.64 (2H, m), 9.31 (1H, s, br.), 9.92 (1H, s) 13.09 (1H, s, br.) ppm.

### EXAMPLE 40

### 7-(3-Trifluoromethyl-2-pyridyl)-N-(5-trifluoromethyl-2-pyridyl)[1,2,4]triazolo[4,3-b]pyridazine-3-amine

A mixture of Description 4 (0.25 g, 0.88 mmol), tris(dibenzylidene)dipalladium (0.016 g, 0.018 mmol), 2-bromo-5-trifluoromethylpyridine (0.22 g, 0.97 mmol) and cesium carbonate (0.41 g, 1.26 mmol) in anhydrous 1,4-dioxane (5 ml) was degassed by bubbling nitrogen through for 10 min. The mixture was heated at 100° C. for 15 h, allowed to cool to room temperature and poured into a mixture of ethyl acetate/water (20/5 ml). The phases were separated and the aqueous phase was extracted two times with ethyl acetate (10 ml each). The combined organic layers were washed with brine, dried over sodium sulfate and adsorbed onto silica gel. Purification by flash chromatography (ethyl acetate/iso-hexane=1:1) gave the title compound (0.18 g, 48%) as a canary-yellow solid, MS: (ES (M+1)) 426.

¹H NMR (360 MHz, DMSO) δ 7.47 (1H, d, J=8.7 Hz), 7.79-7.83 (1H, m), 8.05-8.07 (1H, m), 8.44-8.50 (3H, m), 8.75 (1H, d, J=1.4 Hz), 9.02 (1H, d, J=1.4 Hz), 10.51 (1H, s) ppm.

### EXAMPLE 41

### 7-(3-Chloro-2-pyridyl)-N-(4-trifluoromethylphenyl)[1,2,4]triazolo[4,3-b]pyridazine-3-amine

Using a procedure analogous to Example 1 but starting with 3-chloro-5-(3-chloro-2-pyridyl)pyridazine (obtained in a similar way to Description 1) the title compound was obtained (0.32 g, 38%) as a bright yellow solid, MS: (ES (M+1)) 391.

¹H NMR (360 MHz, DMSO) δ 7.59 (1H, dd, J=8.2 and 4.7), 7.70 (2H, d, J=8.7), 8.07 (2H, d, J=8.7), 8.18 (1H, d, J=8.2), 8.63 (1H, d, J=1.8), 8.73 (1H, d, J=4.7), 8.89 (1H, d, J=1.8), 10.32 (1H, s) ppm.

### EXAMPLE 42

### 7-(3-Bromo-2-pyridyl)-N-(4-trifluoromethylphenyl)[1,2,4]triazolo[4,3-b]pyridazine-3-amine

Using a procedure analogous to Example 1 but starting with 3-chloro-5-(3-bromo-2-pyridyl)pyridazine (obtained in a similar way to Description 1) the title compound was obtained (0.35 g, 45%) as a bright yellow solid, MS: (ES (M+1)) 435.

¹H NMR (360 MHz, DMSO) δ 7.50 (1×, dd, J=8.1 and 4.6), 7.70 (2H, d, J=8.6), 8.07 (2H, d, J=8.6), 8.33 (1H, d, J=8.1), 8.59 (1H, d, J=1.8), 8.76 (1H, d, J=4.6), 8.87 (1H, d, J=1.8), 10.32 (1H, s) ppm.

### EXAMPLE 43

### 7-[3-(1,3-Thiazol-2-yl)pyridin-2-yl]-N-(4-trifluoromethylphenyl)[1,2,4]triazolo[4,3-b]pyridazine-3-amine

A mixture of Example 42 (0.11 g, 0.25 mmol), 2-(tri n-butylstannyl)thiazole (0.12 g, 0.32 mmol), copper(I) iodide (0.005 g, 0.026 mmol), tetrakis(triphenylphosphino)palladium(0) (0.015 g, 0.013 mmol) and lithium chloride (0.032 g, 0.75 mmol) was suspended in dioxane (2 ml), using a Personal Chemistry process vial. After capping the vial it was irradiated in a Personal Chemistry Smith system at 160° C. for 10 min and cooled to room temperature. The resulting black suspension was poured onto a mixture of chloroform and water (20/10 ml) and the phases were separated. After two further extractions the combined organic extracts were washed with water and brine and dried over sodium sulfate. After filtration the compound was adsorbed onto silica gel and purified by flash column (50% ethyl acetate) to yield the title compound (0.06 g, 55%) as a canary-yellow solid, MS: (ES (M+1)) 440.

¹H NMR (360 MHz, DMSO) δ 7.68-7.73 (3H, m), 7.91 (2H, s), 8.04 (2H, d, J=8.6 Hz), 8.25 (1H, d, J=1.9), 8.34 (1H, d, J=7.9), 8.45 (1H, d, J=1.9), 8.87 (1H, d, J=4.6), 10.25 (1H, s) ppm.

### EXAMPLE 44

### 7-[3-(1-Methyl-1H-pyrazol-5-yl)pyridin-2-yl]-N-(4-trifluoromethyl-Phenyl)[1,2,4]triazolo[4,3-b]pyridazin-3-amine

Using a procedure analogous to Example 43 but starting with 2-methyl-3-(tri n-butylstannyl)pyrazole the title compound was obtained (0.04 g, 21%) as a bright yellow solid, MS: (ES (M+1)) 437.

¹H NMR (360 MHz, DMSO) δ 3.59 (3H, a), 6.38 (1H, d, J=1.8 Hz), 7.52 (1H, d, J=1.8 Hz), 7.66-7.69 (3H, m), 7.90 (1H, d, J=2.0 Hz), 8.03-8.07 (3H, m), 8.49 (1H, d, J=2.0 Hz), 8.89 (1H, d, J=4.8 Hz), 10.24 (1H, s) ppm.

### EXAMPLE 45

### 7-(3-Ethoxycarbonyl-2-pyridyl)-N-(4-trifluoromethylphenyl)[1,2,4]triazolo[4,3-b]pyridazine-3-amine

Using a procedure analogous to Example 1 but starting with 5-(3-ethoxycarbonyl-2-pyridyl)-3-chloropyridazine (obtained in a similar way to Description 1) the title compound was obtained (0.14 g, 25%) as a bright yellow solid, MS: (ES (M+1)) 429.

¹H NMR (360 MHz, DMSO) δ 1.17 (3H, tr, J=7.1), 4.24 (2H, q, J=7.1), 7.69-7.72 (3H, m), 8.06 (2H, d, J=8.6), 8.33 (1H, d, J=1.8), 8.43 (1H, dd, J=7.9 and 1.5), 8.78 (1H, d, J=1.5), 8.94 (1H, d, J=1.8), 10.28 (1H, s) ppm.

### EXAMPLE 46

### 7-(3-Cyano-2-pyridyl)-N-4-trifluoromethylphenyl[1,2,4]triazolo[4,3-b]-pyridazine 3-amine

Using a procedure analogous to Example 1 but starting with 3-chloro-5-(3-cyano-2-pyridyl)pyridazine (obtained in a similar way to Description 1) the title compound was obtained (0.10 g, 21%) as a bright yellow solid, MS: (ES (M+1)) 382.

¹H NMR (360 MHz, DMSO) δ 7.70 (2H, d, J=8.7), 7.76 (1H, dd, J=7.9 and 4.9), 8.08 (2H, d, J=8.7), 8.57 (1H, d, J=7.9), 8.82 (1H, d, J=1.8), 8.97 (1H, d, J=1.8), 9.04 (1H, d, J=4.9), 10.37 (1H, s) ppm.

Examples 47-53 were made by a procedure analogous to Example 40 using the indicated starting materials.

### EXAMPLE 47

### N-(4-Chlorophenyl)-7-(3-trifluoromethyl-2-pyridyl)[1,2,4]triazolo[4,3-b]pyridazine-3-amine

4-chloroiodobenzene gave the title compound (0.02 g, 8%) as a bright yellow solid, MS: (ES (M+1)) 391.

¹H NMR (500 MHz, DMSO) δ 7.40 (2H, d, J=8.7), 7.81 (1H, dd, J=8.0 and 4.9), 7.93 (2H, d, J=8.7), 8.33 (1H, d, J=1.8), 8.44 (1H, d, J=8.0), 8.72 (1H, d, J=1.8), 9.02 (1H, d, J=4.9), 9.99 (1H, s) ppm.

### EXAMPLE 48

### N-(4-Tolyl)-7-(3-trifluoromethyl-2-pyridyl)[1,2,4]triazolo[4,3-b]pyridazine-3-amine

4-Bromotoluene gave the title compound (0.015 g, 10%) as a bright yellow solid, MS: (ES (M+1)) 371.

¹H NMR (400 MHz, CDCl₃) δ 2.36 (3H, s), 7.23 (2H, d, J=8.4), 7.46 (1H, s, br), 7.53 (2H, d, J=8.4), 7.65 (1H, dd, J=8.2 and 5.2), 8.23 (1H, d, J=8.2), 8.62 (1H, d, J=1.8), 8.66 (1H, d, J=1.8), 8.96 (1H, d, J=5.2) ppm.

### EXAMPLE 49

### N-(4-(2-Hydroxyethyl)phenyl)-7-(3-trifluoromethyl-2-pyridyl)[1,2,4]triazolo[4,3-b]pyridazine-3-amine

2-(4-Bromophenyl)ethanol gave the title compound (0.024 g, 6%) as a bright yellow solid, MS: (ES (M+1)) 401.

¹H NMR (400 MHz, CDCl₃) δ 2.87 (2H, t, J=6.4), 3.90 (2H, t, J=6.4), 5.11 (2H, s, br), 7.25 (2H, d, J=8.5), 7.58 (2H, d, J=8.5), 7.63 (1H, dd, J=8.1 and 5.1), 8.22 (1H, d, J=8.1), 8.49 (1H, d, J=1.8), 8.61 (1H, d, J=1.8), 8.95 (1H, d, J=5.1) ppm.

### EXAMPLE 50

### N-(4-Methylsulfonylphenyl)-7-(3-trifluoromethyl-2-pyridyl)[1,2,4]triazolo[4,3-b]pyridazine-3-amine

4-Bromophenyl methyl sulfone gave the title compound (0.008 g, 9%) as a bright yellow solid, MS: (ES (M+1)) 435.

¹H NMR (400 MHz, CDCl₃) δ 3.08 (3H, s), 7.45 (1H, s, br), 7.61 (1H, dd, J=7.6 and 4.4), 7.99-8.01 (4H, m), 8.20-8.22 (2H, m), 8.54 (1H, d, J=1.9), 8.95 (1H, d, J=4.4) ppm.

### EXAMPLE 51

### N-(2-Chloro-5-pyridyl)-7-(3-trifluoromethyl-2-pyridyl)[1,2,4]triazolo[4,3-b]pyridazine-3-amine

2-Chloro-5-iodopyridine gave the title compound (0.014 g, 5%) as a bright yellow solid, MS: (ES (M+1)) 392.

¹H NMR (500 MHz, DMSO) δ 7.73 (1H, d, J=5.1), 8.25 (1H, m), 8.58 (1H, d, J=1.8), 8.63-8.70 (2H, m), 8.99 (1H, s), 9.15 (1H, d, J=1.8), 9.28 (1H, d, J=5.1), 10.59 (1H, s) ppm.

### EXAMPLE 52

### N-(4-(1-Cyano-1-methylethyl)phenyl)-7-(3-trifluoromethyl-2-pyridyl)[1,2,4]triazolo[4,3-b]pyridazine-3-amine

2-(4-Bromophenyl)-2-methylpropionitrile gave the title compound (0.070 g, 32%) as a bright yellow solid, MS: (ES (M+1)) 424.

¹H NMR (360 MHz, CDCl₃) δ 1.75 (6H, s), 7.15 (1H, s), 7.51 (2H, d, J=8.8), 7.57-7.60 (1H, m), 7.82 (2H, d, J=8.8), 8.15 (1H, d, J=1.9), 8.19 (1H, d, J=5.2) 8.49 (1H, d, J=1.9), 8.94 (1H, d, J=5.2) ppm.

### EXAMPLE 53

### N-(4-(1-cyclopropylphenyl)-7-(3-trifluoromethyl-2-pyridyl)[1,2,4]triazolo[4,3 b]pyridazine-3-amine

1-(4-Bromophenyl)-1-cyclopropanecarbonitrile gave the title compound (0.076 g, 62%) as a bright yellow solid, MS: (ES (M+1)) 422.

¹H NMR (500 MHz, CDCl₃) δ 1.40 (2H, m), 1.71 (2H, m), 7.13 (1H, s), 7.35 (2H, d, J=8.7), 7.58-7.60 (1H, m), 7.80 (2H, d, J=8.7), 8.15 (1H, d, J=1.9), 8.19 (1H, d, J=5.0), 8.49 (1H, d, J=1.9), 8.93 (1H, d, J=5.0) ppm. CL Example 54

### N-(4-Bromophenyl)-7-(3-trifluoromethyl-2-pyridyl)[1,2,4]triazolo[4,3-b]pyridazine-3-amine

Using a procedure analogous to Example 1 but starting with N-4-bromophenylisocyanate the title compound was obtained (1.01 g, 46%) as a bright yellow solid, MS: (ES (M+1)) 435/437.

¹H NMR (400 MHz, DMSO) δ 7.52 (2H, d, J=8.9), 7.81 (1H, dd, J=8.0 and 4.6), 7.88 (2H, d, J=8.9), 8.34 (1H, d, J=1.8), 8.44 (1H, d, J=8.0), 8.72 (1H, d, J=1.8), 9.04 (1H, d, J=4.6), 10.01 (1H, s) ppm.

### EXAMPLE 55

### N-(4-(2-Methyl-3-pyrazolo)phenyl)-7-(3-trifluoromethyl-2-pyridyl)[1,2,4]triazolo[4,3-b]pyridazine-3-amine

Using a procedure analogous to Example 43 but starting with Example 54 and 2-methyl-3-(tri n-butylstannyl)pyrazole the title compound was obtained (0.06 g, 29%) as a bright yellow solid, MS: (ES (M+1)) 437.

¹H NMR (360 MHz, DMSO) δ 3.87 (3H, s), 6.37 (1H, d, J=1.8 Hz), 7.45 (1H, d, J=1.8 Hz), 7.52 (2H, d, J=8.7 Hz), 7.81 (1H, dd, J=8.1 and 4.6 Hz), 8.01 (2H, d J=8.7 Hz), 8.35 (1H, d, J=1.5 Hz), 8.45 (1H, d, J=8.1 Hz), 8.75 (1H, d, J=1.5 Hz), 9.04 (1H, d, J=4.6 Hz), 10.05 (1H, s) ppm.

### EXAMPLE 56

### N-(4-(4-Fluorophenyl)phenyl)-7-(3-trifluoromethyl-2-pyridinyl)[1,2,4]triazolo[4,3-b]pyridazine-3-amine

Using a procedure analogous to Example 43 but starting with Example 54 and 4-fluoro-1-(tri n-butylstannyl)benzene the title compound was obtained (0.14 g, 66%) as a bright yellow solid, MS: (ES (M+1)) 451.

¹H NMR (360 MHz, DMSO) δ 7.25-7.30 (2H, m), 7.65-7.73 (4H, m), 7.80-7.84 (1H, m), 8.00 (2H, d J=6.8 Hz), 8.34 (1H, d, J=1.8 Hz), 8.45 (1H, d, J=8.2 Hz), 8.74 (1H, d, J=1.8 Hz), 9.04 (1H, d, J=4.0 Hz), 9.98 (1H, s) ppm.

Examples 57 to 61 were made using a procedure analogous to Example 1 with the indicated starting materials.

### EXAMPLE 57

### N-Butyl-7-(3-trifluoromethyl-2-pyridyl)[1,2,4]triazolo[4,3-b]pyridazine-3-amine

N-Butylisocyanate gave the title compound (0.14 g, 21%) as a bright yellow solid, MS: (ES (M+1)) 337.

¹H NMR (360 MHz, DMSO) δ 0.93 (3H, tr, J=7.4), 1.33-1.44 (2H, m), 1.62-1.71 (2H, m), 3.37-3.46 (2H, m), 5.87 (2H, tr, J=5.9), 7.78 (1H, dd, J=8.1 and 4.9), 8.14 (1H, d, J=1.8), 8.42 (1H, d, J=8.1), 8.54 (1H, d, J=1.8), 9.01 (1H, d, J=4.9) ppm.

### EXAMPLE 58

### N-(1-Adamantyl)-7-(3-trifluoromethyl-2-pyridyl)[1,2,4]triazolo[4,3-b]pyridazine-3-amine

N-(1-Adamantyl)isocyanate gave the title compound was obtained (0.023 g, 15%) as a bright yellow solid, MS: (ES (M+1)) 415.

¹H NMR (400 MHz, CDCl₃) δ 1.72-1.81 (6H, m), 2.19-2.22 (9H, m), 4.74 (1H, s), 7.54 (1H, dd, J=7.8 and 5.2), 8.03 (1H, d, J=1.8), 8.16 (1H, d, J=7.8), 8.33 (1H, d, J=1.8), 8.90 (1H, d, J=5.2) ppm.

### EXAMPLE 59

### N-(1-Trifluoroacetyl-4-piperidinyl)-7-(3-trifluoromethyl-2-pyridyl)[1,2,4]triazolo[4,3-b]pyridazine-3-amine

N-(1-Trifluoroacetyl-4-piperidinyl)isocyanate gave the title compound (0.14 g, 31%) as a bright yellow solid, MS: (ES (M+1)) 460

¹H NMR (400 MHz, CDCl₃) δ 1.3-1.75 (2H, m, br.), 2.38-2.46 (2H, m, br.), 3.09-3.16 (1H, m, br.), 3.37-3.44 (1H, m, br.), 4.06-4.15 (1H, m, br.), 4.34-4.37 (1H, m, br.), 4.52-4.56 (1H, m, br.), 4.90-5.15 (1H, br.), 7.57 (1H, dd, J=7.8 and 5.2), 8.07 (1H, d, J=1.9), 8.17 (1H, d, J=7.8), 8.41 (1H, d, J=1.9), 8.93 (1H, d, J=5.2) ppm.

### EXAMPLE 60

### N-(1-Cyclohexyl)-7-(3-trifluoromethyl-2-pyridyl)[1,2,4]triazolo[4,3-b]pyridazine-3-amine

N-Cyclohexylisocyanate gave the title compound (0.022 g, 6%) as a bright yellow solid, MS: (ES (M+1)) 363.

¹H NMR (360 MHz, CDCl₃) δ 1.19-1.53 (5H, m), 1.67-1.71 (1H, m), 1.81-1.83 (2H, m), 2.23-2.27 (2H, m), 3.94-4.01 (1H, m), 4.70 (1H, d, J=7.7), 7.54 (1H, dd, J=7.8 and 5.2), 8.02 (1H, d, J=1.8), 8.16 (1H, d, J=7.8), 8.34 (1H, d, J=1.8), 8.91 (1H, d, J=5.2) ppm.

### EXAMPLE 61

### N-(1-Phenyl-4-piperidinyl)-7-(3-trifluoromethyl-2-pyridyl)[1,2,4]triazolo[4,3-b]pyridazine-3-amine

N-(1-Phenyl-4-piperidinyl)isothiocyanate gave the title compound (0.05 g, 17%) as a bright yellow solid, MS: (ES (M+1)) 440

¹H NMR (400 MHz, CDCl₃) δ 1.77-1.87 (2H, m), 2.38-2.40 (2H, m), 2.98-3.04 (2H, m), 3.71-3.74 (2H, m), 4.13-4.20 (1H, m), 4.76 (1H, d, J=7.8), 6.86 (1H, tr., J=7.3), 6.98 (2H, d, J=7.9), 7.27 (2H, d, J=7.9), 7.55 (1H, dd, J=8.0 and 4.6), 8.04 (1H, d, J=1.8), 8.16 (1H, d, J=8.0), 8.36 (1H, d, J=1.8), 8.92 (1H, d, J=4.6) ppm.

### EXAMPLE 62

### N-(4-Trifluoromethylphenyl-7-(2-cyanophenyl)-[1,2,4]triazolo[4,3-b]pyridazine-3-amine

To a mixture of 3-chloro-5-(2-cyanophenyl)pyridazine (obtained similarly to Description 1, 0.38 g, 1.76 mmol) in anhydrous isopropanol (10 ml) was added hydrazine monohydrate (0.4 ml, 8.3 mmol) and the mixture was heated at 100° C. for 15 h. After cooling to room temperature the solution was concentrated under reduced pressure and toluene was added to the resulting oil. The mixture was concentrated under reduced pressure again and the whole procedure was repeated twice to yield 5-(2-cyanophenyl)-3-hydrazinopyridazine (0.35 g, 95%) as a red syrup. Using a procedure analogous to Example 1 but starting with N-4-trifluoromethylphenylisocyanate the title compound was obtained (0.16 g, 25%) as a bright yellow solid, MS: (ES (M+1)) 381.

¹H NMR (500 MHz, DMSO) δ 7.69-7.74 (3H, m), 7.87-7.93 (2H, m), 8.06-8.08 (3H, m), 8.56 (1H, d, J=1.8), 8.89 (1H, d, J=1.8), 10.31 (1H, s) ppm.

### EXAMPLE 63

### N-(4-Trifluoromethylphenyl)-7-(3-(1-hydroxy-1-methylethyl)-2-pyridyl-[1,2,4]triazolo[4,3-b]pyridazine-3-amine

To a mixture of 3-chloro-5-(3-acetyl-2-pyridyl)pyridazine (obtained using 3-acetyl-2-chloropyridine similarly to Description 1, 2.25 g, 9.64 mmol) in anhydrous tetrahydrofuran (60 ml) was added methyl magnesium chloride (3.53 ml of 3.0M solution in tetrahydrofuran, 10.6 mmol) at −40° C. under nitrogen and the dark red mixture was warmed up to 0° C. over 15 h. After addition of saturated ammonium chloride solution (10 ml) the mixture was extracted with ethyl acetate (3 times 50 ml) and the combined organic fractions were washed with brine and dried over sodium sulfate. The drying agent was filtered off and the solution concentrated under reduced pressure to yield 3-chloro-5-(3-(1-hydroxy-1-methylethyl)-2-pyridyl)pyridazine (2.2 g, 91%) as a dark red solid, MS: (ES (m+1)) 250/252. The pyridazine (0.82 g, 3.3 mmol) was reacted with hydrazine hydrate (0.8 ml, 16.5 mmol) according to Example 1 to yield 3-hydrazino-5-(3-(1-hydroxy-1-methylethyl)-2-pyridyl)pyridazine (0.8 g, 99%) as a dark red syrup, MS: (ES (M+1)) 246.

This pyridazine (0.8 g, 3.3 mmol) was dissolved in dry acetonitrile (2 ml) and 4-trifluoromethylphenylisothiocyanate (0.66 g, 3.3 mmol) was added in one portion while stirring at room temperature. The solution was stirred at room temperature for 15 h. Water (5 ml) was added and the obtained solid filtered and dried on the sinter to yield a beige solid which was suspended in acetonitrile (2 ml), using a Personal Chemistry process vial. Silver(I)acetate (0.55 g, 3.3 mmol) was added and after capping the vial it was irradiated in the Personal Chemistry Smith synthesizer at 150° C. for 10 min and cooled to room temperature. The black mixture was poured onto a mixture of chloroform and water (20/10 ml) and the phases were separated. After two further extractions the combined organic extracts were washed with water and brine and dried over sodium sulfate. After filtration the compound was adsorbed onto silica gel and purified by flash column (ethyl acetate) to yield the title compound (0.69 g, 55%) as a canary yellow solid, MS: (ES(M+1)) 415.

¹H NMR (360 MHz, DMSO) δ 1.49 (6H, s), 5.14 (1H, s), 7.49 (1H, dd, J=8.1 and 4.6 Hz), 7.69 (2H, d, J=8.7), 7.98-8.06 (4H, m), 8.53 (1H, d, J=1.8 Hz), 8.57 (1H, d, J=4.6 Hz), 10.20 (1H, s) ppm.

### EXAMPLE 64

### N-(4-(1-Methylethyl)phenyl)-7-(3-trifluoromethyl-2-pyridyl)[1,2,4]triazolo[4,3-b]pyridazine-3-amine

To a solution of Description 2 (7.6 g, 29.3 mmol) in anhydrous isopropanol (50 ml) was added hydrazine monohydrate (7 ml, 144 mmol) and the mixture was heated at 100° C. for 15 h. After cooling to room temperature the solution was concentrated under reduced pressure and toluene was added to the resulting oil. The mixture was concentrated under reduced pressure again and the whole procedure was repeated twice to yield 3-hydrazino-5-(3-trifluoromethyl-2-pyridyl)pyridazine (6.1 g, 82%) as a red syrup which crystallises over 3 days at room temperature.

The pyridazine (3.5 g, 13.7 mmol) was dissolved in formic acid (95%, 40 ml) and heated at 80° C. for 0.5 h. After cooling to room temperature the red mixture was concentrated under reduced pressure and partitioned between chloroform and aqueous saturated sodium carbonate solution (150/50 ml). After two further extractions the combined organic extracts were washed with brine and dried over sodium sulfate. After filtration the compound was adsorbed onto silica gel and purified by flash column (ethyl acetate) to yield 7-(3-trifluoromethyl-2-pyridyl)[1,2,4]-triazolo[4,3-b]pyridazine (2.8 g, 77%) as an off white solid, MS: (ES (M+1)) 266.

Sodium acetate (2.9 g, 35.3 mmol) was added to a solution of this pyridazine (4.7 g, 17.7 mmol) in glacial acetic acid (40 ml) and a solution of bromine (2.7 ml, 52.7 mmol) in 10 ml glacial acetic acid was added dropwise while stirring at room temperature. The solution was heated at 120° C. for 2 h, cooled to room temperature and most of the acetic acid removed under reduced pressure. Water (200 ml) was added and the obtained precipitate was washed with water followed by diethyl ether. Drying on a sinter yielded 3-bromo-7-(3-trifluoromethyl-2-pyridyl)-1,2,4-triazolo[4,3-b]pyridazine (6.0 g, 100%) as a canary-yellow solid, MS: (ES (M+1)) 344/346.

This pyridazine (0.05 g, 0.14 mmol) and 4-isopropylaniline (0.095 g, 0.7 mmol) were suspended in dioxane (2 ml), using a process vial from Personal Chemistry. After addition of 1 drop HBr (48% in water) the vial was capped and irradiated using the Personal Chemistry Smith synthesizer for 15 min at 190° C. The contents were partitioned between chloroform and water (30/10 ml). After two further extractions the combined organic extracts were washed with brine and dried over sodium sulfate. After filtration the compound was adsorbed onto silica gel and purified by flash column (ethyl acetate) to yield the title compound (0.01 g, 16%) as a canary yellow solid, MS: (ES (M+1)) 399.

¹H NMR (500 MHz, CDCl₃) δ 1.26 (6H, d, J=6.9), 2.92 (1H, sept., J=6.9), 7.01 (1H, s), 7.27 (2H, d, J=8.6), 7.57 (1H, dd, J=7.5 and 4.3), 7.70 (2H, d, J=8.6), 8.13 (1H, d, J=1.8), 8.18 (1H, d, J=7.5), 8.46 (1H, d, J=1.8), 8.93 (1H, d, J=4.3) ppm.

Examples 65-71 were made using a procedure analogous to Example 64 with the indicated starting products.

### EXAMPLE 65

### N-(4-(1-Ethoxycarbonyl-1-methylethyl)phenyl)-7-(3-trifluoromethyl-2-pyridyl)[1,2,4]triazolo[4,3-b]pyridazine-3-amine

Ethyl 2-(4-aminophenyl)-2-methyl propionate gave the title compound was obtained (0.018 g, 13%) as a bright yellow solid, MS: (ES (M+1)) 471

¹H NMR (360 MHz, CDCl₃) δ 1.19 (3H, tr., J=7.1), 1.56 (6H, s), 4.13 (2H, d, J=7.1), 7.06 (1H, s), 7.39 (2H, d, J=8.7), 7.57 (1H, dd, J=8.7 and 5.5), 7.74 (2H, d, J=8.7), 8.13 (1H, d, J=1.8), 8.18 (1H, d, J=8.7), 8.47 (1H, d, J=1.8), 8.92 (1H, d, J=5.5) ppm.

### EXAMPLE 66

### N-(4-Cyclohexylphenyl)-7-(3-trifluoromethyl-2-pyridyl)[1,2,4]triazolo[4,3-b]pyridazine-3-amine

4-Cyclohexylaniline gave the title compound was obtained (0.02 g, 16%) as a bright yellow solid, MS: (ES (M+1)) 439

¹H NMR (400 MHz, CDCl₃) δ 1.37-1.45 (4H, m, br.), 1.73-1.78 (1H, m, br.), 1.81-1.93 (5H, m, br.), 2.45-2.52 (1H, m, br.), 7.11 (1H, s), 7.24 (2H, d, J=8.5), 7.58 (1H, dd, J=7.5 and 4.3), 7.71 (2H, d, J=8.5), 8.13 (1H, d, J=1.8), 8.18 (1H, d, J=7.5), 8.46 (1H, d, J=1.8), 8.93 (1H, d, J=4.3) ppm.

### EXAMPLE 67

### N-(4-(1-Hydroxy-1-trifluoromethyl-2,2,2-trifluoroethyl)phenyl)-7-(3-trifluoromethyl-2-pyridyl)[1,2,4]triazolo[4,3-b]pyridazine-3-amine

1-(4-Aminophenyl)-1-trifluoromethyl-2,2,2-trifluoroethanol gave the title compound was obtained (0.003 g, 2%) as a bright yellow solid, MS: (ES (M+1)) 523

¹H NMR (360 MHz, DMSO) δ 7.64 (2H, d, J=8.5), 7.81 (1H, dd, J=7.5 and 4.5), 7.95 (2H, d, J=8.5), 8.35 (1H, d, J=1.8), 8.43 (1H, d, J=7.5), 8.73 (1H, d, J=1.8), 9.04 (1H, d, J=4.5), 10.12 (1×, 9) ppm.

### EXAMPLE 68

### N-(4-(1-Hydroxy-2-methyl-2-propyl)phenyl)-7-(3-trifluoromethyl-2-pyridyl)[1,2,4]triazolo[4,3-b]pyridazine-3-amine

2-(4-Aminophenyl)-2-methyl-propanol gave the title compound was obtained (0.03 g, 25%) as a bright yellow solid, MS: (ES (M+1)) 429

¹H NMR (360 MHz, CDCl₃) δ 1.36 (6H, s), 3.62 (2H, m), 7.10 (1H, s), 7.43 (2H, d, J=8.5), 7.57 (1H, dd, J=7.5 and 4.5), 7.75 (2H, d, J=8.5), 8.13 (1H, d, J=1.8), 8.18 (1H, d, J=7.5), 8.46 (1H, d, J=1.8), 8.94 (1H, d, J=4.5) ppm.

### EXAMPLE 69

### N-(2-4-Trifluoromethylphenylethyl-7-(3-trifluoromethyl-2-pyridyl)[1,2,4]triazolo[4,3-b]pyridazine-3-amine

Trifluoromethylphenylethylamine gave the title compound was obtained (0.045 g, 12%) as a bright yellow solid, MS: (ES (M+1)) 453

¹H NMR (360 MHz, DMSO) δ 3.11 (2H, tr., J=7.0), 3.71 (2H, m), 7.14 (1H, tr., J=5.8), 7.51 (2H, d, J=8.0), 7.65 (2H, d, J=8.0), 7.77 (1H, dd, J=8.1 and 4.5), 8.14 (1H, d, J=1.8), 8.41 (1H, d, J=8.1), 8.54 (1H, d, J=1.8), 9.00 (1H, d, J=4.5) ppm.

### EXAMPLE 70

### N-(trans)-(4-tert.-Butylcyclohexyl)-7-(3-trifluoromethyl-2-pyridyl)[1,2,4]triazolo[4,3-b]pyridazine-3-amine

4-tert-Butyl-cyclohexylamine gave the title compound was obtained (0.15 g, 25%) as a bright yellow solid, MS: (ES (M+1)) 419

¹H NMR (360 MHz, DMSO) δ 0.87 (9H, s) 0.97-1.18 (3H, m), 1.35-1.48 (2H, m), 1.78-1.84 (2H, m), 2.12-2.17 (2H, m), 3.59-3.68 (1H, m), 6.76 (1H, d, J=8.0), 7.78 (1H, dd, J=7.5 and 4.5), 8.12 (1H, d, J=1.8), 8.42 (1H, d, J=7.5), 8.53 (1H, d, J=1.8), 9.00 (1H, d, J=4.5) ppm.

### EXAMPLE 71

### N-(1-Ethoxycarbonyl-4-piperidinyl)-7-(3-trifluoromethyl-2-pyridyl)[1,2,4]triazolo[4,3-b]pyridazine-3-amine

Ethyl 4-amino-1-piperidine carboxylate gave the title compound was obtained (0.12 g, 15%) as a bright yellow solid, MS: (ES (M+1)) 436

¹H NMR (360 MHz, DMSO) δ 1.19 (3H, tr., J=5.5), 1.52-1.61 (2H, m), 2.01-2.05 (2H, m, br.), 2.88-3.04 (2H, m, br.), 3.89-4.08 (5H, m), 6.96 (1H, d, J=7.8), 7.78 (1H, dd, J=8.2 and 5.2), 8.14 (1H, d, J=1.9), 8.42 (1H, d, J=8.2), 8.55 (1H, d, J=1.9), 8.99 (1H, d, J=5.2) ppm.

### EXAMPLE 72

### 7-(4-Methylpyridazin-3-yl)-N-[4-trifluoromethylphenyl][1,2,4]triazolo[4,3-b]pyridazin-3-amine

### a) Ethyl 2-methyl-4-oxobutanoate

Ozone was bubbled into a solution of ethyl 2-methyl-4-pentenoate (50.0 g, 0.352 mol) in DCM (350 ml) whilst maintaining the reaction temperature at −78° C. A blue colour persisted after 7 h at which point nitrogen was bubbled into the mixture until the blue colour had disappeared. Triphenylphosphine (110.7 g, 0.423 mol) was then added and the mixture was allowed to warm to room temperature and stirred overnight. The mixture was concentrated to dryness and the residue diluted with isohexane. The mixture was filtered to remove the precipitated triphenylphosphine oxide. The filtrate was concentrated to dryness. The residue was purified by flash chromatography on silica gel eluting with 10:1 isohexane-ethyl acetate to give a colourless oil (34 g, 67%). ¹H NMR (400 MHz, CDCl₃) δ 9.77 (1H, s), 4.19-4.11 (2H, m), 3.02-2.86 (2H, m), 2.53 (1H, dd, J=4.7, 17.5 Hz), 1.28-1.22 (6H, m) ppm.

### b) 4-Methyl-4,5-dihydropyridazin-3(2H)-one

Hydrazine hydrate (−55% hydrazine, 13.7 g, 0.236 mol) was added dropwise to a solution of the product of step a) (34.0 g, 0.236 mol) in ethanol (300 ml). The mixture was stirred at room temperature for 2 h and then heated under reflux for 20 h. The mixture was cooled to room temperature and concentrated to dryness. The residue was taken up in toluene and concentrated to dryness, repeating this procedure once more. A yellow solid was obtained (25.8 g, 98%). ¹H NMR (400 MHz, CDCl₃) δ 8.59 (1H, s), 7.15 (1H, t, J=3.1 Hz), 2.65-2.41 (2H, m), 2.30-2.22 (1H, m), 1.25 (3H, d, J=7.0 Hz) ppm.

### c) 4-Methylpyridazin-3(2H)-one

A solution of bromine (27.4 ml, 0.535 mol) in glacial acetic acid (70 ml) was added dropwise to a solution of the product of step b) (25.8 g, 0.232 mol) in glacial acetic acid (330 ml) whilst maintaining the temperature at −50° C. The mixture was then heated at 80° C. for 2 h. The mixture was cooled to room temperature and concentrated to dryness. The solid residue was triturated with ether. The solid was then added to saturated NaHCO₃ solution and extracted with ethyl acetate. The organic extract was dried over sodium sulphate, filtered and concentrated to dryness to give pale brown solid. (12.2 g, 48%). ¹H NMR (400 MHz, CDCl₃) δ 11.50 (1H, s), 7.71 (1H, d, J=4.0 Hz), 7.10 (1H, d, J=2.8 Hz), 2.23 (3H, d, J=1.1 Hz) ppm.

### d) 3-Chloro-4-methylpyridazine

The product of step c) (12.2 g, 0.112 mol) in phosphorus oxychloride (125 ml) was heated at 90° C. for 3 h. The mixture was cooled to room temperature and concentrated almost to dryness. The residue was poured into ice/water, made basic by careful addition of 4N NaOH and extracted with ether. The aqueous phase was re-extracted with DCM. The organic phases were combined, washed with water, dried over sodium sulphate, filtered and concentrated to dryness. The residue was purified by flash chromatography on silica gel in 2:1 ethyl acetate-isohexane to give an orange solid (11.2 g, 78%). ¹H NMR (400 MHz, CDCl₃) δ 8.97 (1 H, d, J=4.9 Hz), 7.35 (1H, d, J=4.9 Hz), 2.44 (3H, s) ppm.

### e) 4-Methyl-1′-(tetrahydro-2H-pyran-2-yl)-3,4′-bipyridazin-6′(1′H)-one

A mixture of Description 46 (10.8 g, 50.4 mmol), bis(pinacolato)diboron (14.9 g, 58.8 mmol), potassium acetate (7.8 g, 79.9 mmol) and bis(diphenylphosphino)ferrocenylpalladiumdichloride (2.5 g, 3.4 mmol) in dioxane (100 ml) was degassed. The resultant mixture was stirred and heated at 100° C., under nitrogen, for 15 h. The mixture was cooled to room temperature. The product of step d) (6.5 g, 50.4 mmol), saturated sodium carbonate solution (54 ml) and bis(diphenylphosphino)ferrocenylpalladiumdichloride (2.5 g, 3.4 mmol) were added and the mixture was degassed again. The resultant mixture was stirred and heated at 100° C., under nitrogen, for 18 h. The mixture was cooled to room temperature and diluted with water followed by extraction with ethyl acetate. The organic phase was washed with brine, dried over sodium sulphate, filtered and concentrated to dryness. The residue was purified by flash chromatography on silica gel twice, eluting with 20:1 DCM-2M NH₃ in MeOH on each occasion. A dark brown oil was obtained (3.5 g, 26%). ¹H NMR (400 MHz, CDCl₃) δ 9.11 (1H, d, J=5.2 Hz), 8.23 (1H, d, J=2.2 Hz), 7.43 (1H, d, J=5 Hz), 7.11 (1H, d, J=2.2 Hz), 6.15 (1H, dd, J=2.1, 10.6 Hz), 4.21-4.13 (1H, m), 3.83-8.77 (1H, m), 2.46 (3H, s), 2.30-2.18 (1H, m), 2.12-2.04 (1H, m), 1.81-1.58 (4H, m) ppm.

### f) 6′-Chloro-4-methyl-3,4′-bipyridazine

The product of step 1) (2.9 g, 10.6 mmol) in phosphorus oxychloride (20 ml) was heated at 85° C. for 5 min. The mixture was cooled to room temperature and added to ice. The pH was adjusted to ˜10 by the careful addition of 4N NaOH and the mixture was then extracted with DCM. The organic extract was dried over sodium sulphate, filtered and concentrated to dryness. The residue was purified by flash chromatography on silica gel eluting with 20:1 DCM-MeOH to give a brown solid which was subsequently triturated with ether to give a light brown solid (770 mg, 35%). ¹H NMR (400 MHz, CDCl₃) δ 9.42 (1H, d, J=1.8 Hz), 9.17 (1H, d, J=5.3 Hz), 7.87 (1H, d, J=1.8 Hz), 7.49 (1H, d, J=5.2 Hz), 2.50 (3H, s) ppm.

### g) 6′-Hydrazino-4-methyl-3,4′-bipyridazine

The product of step f) (770 mg, 3.72 mmol) and hydrazine hydrate (˜55% hydrazine, 1.1 ml, 18.6 mmol) in propan-2-ol (5 ml) were heated at 100° C. for 18 h. The mixture was cooled to room temperature and concentrated to dryness. The residue was diluted with toluene and concentrated to dryness again. The crude product was triturated with ether and further purified by passing through an SCX cartridge to give a brown solid (188 mg, 37%). ¹H NMR (400 MHz, DMSO) δ 9.16 (1H, d, J=5.2 Hz), 8.70 (1H, d, J=1.8 Hz), 8.15 (1H, s), 7.71 (1H, d, J=4.8 Hz), 7.23 (1H, m), 4.38 (2H, s), 2.37 (3H, s) ppm.

### h) 7-(4-Methylpyridazin-3-yl)-N-[4-trifluoromethylphenyl][1,2,4]triazolo[4,3-b]pyridazin-3-amine

The product step g) (265 mg, 1.31 mmol) and 4-trifluoromethylphenyl isocyanate (245 mg, 1.31 mmol) in acetonitrile (5 ml) were stirred at room temperature for 18 h. Phosphorus oxychloride (1.22 ml) was then added and the mixture was stirred and heated at 95° C. for 48 h. On cooling to room temperature the mixture was added to ice/water and basified with aqueous NaOH. The resultant mixture was extracted with DCM. The organic phase was dried over sodium sulphate, filtered and concentrated to dryness. The residue was purified by flash chromatography on silica gel eluting with 10:1 DCM-MeOH to give the title compound as an orange solid which was triturated with ethyl acetate to give a yellow solid (133 mg, 27%). ¹H NMR (400 MHz, DMSO) δ 10.33 (1H, s), 9.19 (1H, d, J=5.2 Hz), 8.89 (1H, d, J=1.9 Hz), 8.61 (1H, d, J=1.9 Hz), 8.08 (2H, d, J=8.6 Hz), 7.77 (1H, d, J=5.2 Hz), 7.70 (2H, d, J=8.7 Hz), 2.53 (3H, s) ppm, MS: (ES (M+1)) 372.

### EXAMPLE 73

### N-[4-trifluoromethylphenyl]-7-[4-trifluoromethylpyridazin-3-yl][1,2,4]triazolo[4,3-b]pyridazin-3-amine

### a) Ethyl 3,3,3-trifluoropropanoate

Oxalyl choride (34.1 ml, 390 mmol) was added dropwise to a stirred solution of 3,3,3-trifluoropropionic acid (50.0 g, 390 mmol) and DMP (5 drops) in DCM (400 ml). The mixture was stirred at room temperature for 18 h. Pyridine (31.6 ml, 390 mmol) followed by ethanol (21.6 g, 469 mmol) were then added. The mixture was stirred at room temperature for 18 h and diluted with DCM. The DCM solution was washed with 2M HCl, water, saturated sodium hydrogen carbonate solution and brine. The organic phase was dried over sodium sulphate, filtered and concentrated to dryness to give an orange oil (35.8 g, 59%). ¹H NMR (400 MHz, CDCl₃) δ 4.24 (2H, q, J=7.1 Hz), 3.17 (2H, q, J=10.1 Hz), 1.30 (3H, t, J=7.1 Hz) ppm.

### b) Ethyl 2-trifluoromethylpent-4-enoate

The product step a) (51.4 g, 329 mmol), allyl methyl carbonate (40.1 g, 346 mmol), BINAP (20 g, 32.1 mmol), 5 Å molecular sieves (150 g) and tris(dibenzylidineacetone)dipalladium(0) chloroform adduct (8.3 g, 8.0 mmol) in THF (750 ml) were stirred and heated at 50° C. for 6 h. The mixture was cooled to room temperature, diluted with ether and filtered through Celite. The filtrate was concentrated to dryness and the residue purified by flash chromatography on silica gel eluting with 50:1 isohexane-ether to give an orange oil (22.5 g, 35%). ¹H NMR (400 MHz, CDCl₃) δ 5.79-5.69 (1H, m), 5.18-5.10 (2H, m), 4.23 (2H, q, J=7.1 Hz), 3.23-3.15 (1H, m), 2.69-2.51 (2H, m), 1.28 (3H, t, J=7.2 Hz) ppm.

### c) Ethyl 4-oxo-2-trifluoromethylbutanoate

Ozone was bubbled into a solution of the product of step b) (21.5 g, 110 mmol) in DCM (150 ml) whilst maintaining the reaction temperature at −78° C. After 3 h a blue colour persisted. Nitrogen was bubbled into the mixture until the blue colour had disappeared. Triphenylphosphine (50.0 g, 191 mmol) was added and the mixture was allowed to warm and stirred at room temperature overnight. The mixture was concentrated and purified by flash chromatography on silica gel in 5:1 isohexane to give a yellow liquid (7.7 g, 35%).

¹H NMR (400 MHz, CDCl₃) δ 9.77 (1H, s), 4.30-4.22 (2H, m), 3.75-3.65 (1H, m), 3.28 (1H, dd, J=10.3, 18.9 Hz), 2.89 (1H, dd, J=3.5, 18.9 Hz), 1.30 (3H, t, J=7.1 Hz) ppm.

### d) 4-Trifluoromethyl-4,5-dihydropyridazin-3(2H)-one

Carried out using the same procedure as for Description 1 using the product of step c) (6.0 g, 93%). ¹H NMR (400 MHz, CDCl₃) δ 8.54 (1H, 9), 7.20 (1H, m), 3.32-3.20 (1H, m), 2.89-2.73 (2H, m) ppm.

### e) 4-Trifluoromethylpyridazin-3(2H)-one

A solution of bromine (4.26 ml, 36.1 mmol) in glacial acetic acid (20 ml) was added dropwise to a stirred solution of the product of step d) (6.0 g, 36.1 mmol) in glacial acetic acid (80 ml) whilst maintaining the temperature of the mixture at ˜50° C. The mixture was cooled to room temperature and concentrated to dryness. The residue was diluted with toluene and concentrated to dryness again. The crude product was purified by flash chromatography on silica gel in 40:1 DCM:MeOH to give a brown solid (4.6 g, 78%). ¹H NMR (400 MHz, DMSO) δ 13.77 (1H, s), 8.07 (1H, d, J=4.3 Hz), 7.88 (1H, dd, J=0.8, 4.0 Hz) ppm.

### f) 3-Chloro-4-trifluoromethylpyridazine

The product of step e) (1.0 g, 6.1 mmol) in phosphorus oxychoride (10 ml) was stirred and heated at 110° C. for 90 min. The mixture was cooled to room temperature, added to ice and extracted with ethyl acetate. The organic extract was washed with water, saturated sodium hydrogen carbonate solution and brine. The organic phase was then dried over sodium sulphate, filtered and concentrated to dryness. The residue was purified by flash chromatography on silica gel eluting with 3:1 isohexane-ethyl acetate to give a brown liquid (520 mg, 47%). ¹H NMR (400 MHz, CDCl₃) δ 9.38 (1H, d, J=5.2 Hz), 7.78 (1H, d, J=5.2 Hz) ppm.

### g) 1′-(Tetrahydro-2H-pyran-2-yl)-4-trifluoromethyl-3,4′-bipyridazin-6′(1′H)-one

Carried out using the same procedure as in Description 1 using the product of step f) (2.88 g, contained an impurity by NMR but used directly in next step).

### h) 6′-Chloro-4-trifluoromethyl-3,4′-bipyridazine

Carried out using the same procedure as in Description 1 using the product of step g) except the mixture was heated at 85° C. for 1 min only (237 mg). ¹H NMR (400 MHz, CDCl₃) δ 9.62 (1H, d, J=5.3 Hz), 9.38 (1H, s), 7.97 (1H, m), 7.83 (1H, m) ppm.

### i) 6′-Hydrazino-4-trifluoromethyl-3,4′-bipyridazine

The product of step h) (242 mg, 0.927 mmol) and hydrazine hydrate (−55% hydrazine, 0.27 ml, 4.64 mmol) in propan-2-ol (3 ml) were stirred and heated at 70° C. for 4 h. The supernatant liquid was decanted off and concentrated to give a dark red oil. The oil was diluted with a small volume of THF and allowed to stand for 2 days. The supernatant layer was decanted off and concentrated to give a red oil. (197 mg, 83%). ¹H NMR (400 MHz, DMSO) δ 9.68 (1H, m), 8.60 (1H, m), 8.31 (2H, m), 7.21 (1H, m), 4.42 (2H, s) ppm.

### j) N-[4-trifluoromethylphenyl]-7-[4-trifluoromethylpyridazin-3-yl][1,2,4]triazolo[4,3-b]pyridazin-3-amine

Carried out using the same procedure as in Description 1 using the product of step i) (54 mg, 17%). ¹H NMR (400 MHz, DMSO) δ 10.36 (1H, s), 9.74 (1H, d, J=5.2 Hz), 8.85 (1H, s), 8.54 (1H, s), 8.39 (1H, d, J=5.4 Hz), 8.08 (2H, d, J=8.6 Hz), 7.71 (2H, d, J=8.7 Hz) ppm, MS: (ES (M+1)) 426.

### EXAMPLE 74

### N-(4-Trifluoromethylphenyl)-6-(3-trifluoromethylpyrid-2-yl)pyrazolo[1,5-a]pyrimidin-3-amine

a) 6-Bromo-pyrazolo[1,5-a]pyrimidine: Bromomalonaldehyde (12 g, 0.08 mol) and 3-aminopyrazole (6 g, 0.087 mol) in acetic acid (10 ml) and EtOH (150 ml) was heated at reflux for 4 h. After cooling to room temperature the insolubles were removed by filtration and the filtrate concentrated. The residue was partitioned between 1N NaOH solution (50 ml) and EtAc (3×100 ml). The organic phase was dried (MgSO₄) and concentrated to give a yellow gum which was purified by column chromatography (silica; EtAc:Hexane 1:4) to give 6 g of product as pale yellow crystals, MS: (ES (M+1)) 198, 200.

b) 6-(3-Trifluoromethylpyrid-2-yl)pyrazolo[1,5-a]pyrimidine: A mixture of the product of step a) (2 g, 0.01 mol), bispinacolatodiborane (2.5 g, 0.01 mol), PdCl₂(dppf) (80 mg) and KOAc (1.5 g, 0.015 mol) in dioxane (50 ml) was heated at reflux under nitrogen for 48 h. A saturated aqueous solution of sodium carbonate (5 ml), PdCl₂(dppf) (80 mg) and 2-chloro-3-trifluoromethylpyridine (2 g, 0.011 mol) were added and the mixture heated at 110° C. for a further 24 h. After cooling, the mixture was filtered through celite, the filtrate concentrated and the residue purified by column chromatography (silica; EtAc:Hexane 1:3->1:1) to give 1.4 g of product as a yellow oil, MS: (ES (M+1)) 265.

c) 3-Nitro-6-(3-trifluoromethylpyrid-2-yl)pyrazolo[1,5-a]pyrimidine: The product of step b) (1.4 g, 0.0053 mol) was dissolved in cone. H₂SO₄ (5 ml) cooled in ice and a 1:1 mixture of fuming HNO₃ and cone. H₂SO₄ (2 ml) was added drop wise over 5 min. After 30 min the cooling bath was removed and the mixture stirred at room temperature for 2 h. After this time MS showed no remaining starting material. The mixture was poured into ice water and extracted with EtOAc (3×20 ml). The organic phase was dried (MgSO₄) and concentrated to give a gum which was purified by column chromatography (silica; EtAc:Hexane 1:1) to give 0.6 g of product as a reddish solid, MS: (ES (M+1)) 310.

d) 6-(3-Trifluoromethylpyrid-2-yl)pyrazolo[1,5-a]pyrimidin-3-amine: The product of step c) (0.2 g) and Lindlar catalyst (0.1 g) in a mixture of 2:1 mixture of MeOH:EtOAc (15 ml) was shaken on a Parr hydrogenator at 30 psi H₂ for 30 min. The catalyst was removed by filtration, the filtrate concentrated and the residue purified by column chromatography (silica; CH₂Cl₂—>CH₂Cl₂:MeOH:NH₃ 95:5:1) to give 0.12 g of product as a yellow oil, MS: (ES (M+1)) 280.

e) N-(4-(Trifluoromethyl)phenyl)-6-(3-trifluoromethylpyrid-2-yl)pyrazolo[1,5-a]Pyrimidin-3-amine; A mixture of the product of step d) (0.12 g, 0.00036 mol), 4-bromobenzotrifluoride (0.08 g, 0.00036 mol), Cs₂CO₃ (0.18 g, 0.00054 mol), xantphos (19 mg) and Pd(dba)₃ in dioxane (10 ml) was heated at 110° C. under N₂ for 18 h. The mixture was then cooled to room temperature and filtered through celite. The filtrate was concentrated and purified by column chromatography (silica; EtAc:Hexane 1:1) to give 0.03 g of product as a yellow solid, MS: (ES (M+1)), 424. ¹H NMR (360 MHz, DMSO) δ 6.89 (2H, d, J=8.5 Hz), 7.45 (2H, d, J=8.5 Hz), 7.54-7.58 (1H, m), 8.18 (1H, d, J=7.8 Hz), 8.28 (1H, s) 8.63 (1H, d, J=1.9 Hz), 8.85 (1H, d, J=1.9 Hz), 8.94 (1H, J=7.8 Hz) ppm.

### EXAMPLE 75

### 4-Trifluoromethylphenyl-3-(3-trifluoromethylpyridin-2-yl)imidazo[1,5-b]pyridazin-7-ylamine

To a solution of Description 10 (5 mg) in dichloromethane (0.5 ml) was added Burgess reagent (9 mg) in 3 portions over 1 h. After 3 h an additional 3 mg Burgess reagent added. After 6 h the reaction was condensed and the product isolated by gradient column chromatography, eluting with 50% ethyl acetate in hexane-neat ethyl acetate to give the desired nitrile (4 mg). ¹H NMR (400 MHz, CDCl₃) 7.59 (1H, ddd, J 8.2, 4.1, 1.3 Hz), 7.95 (1H, d, J=2.2 Hz), 8.15 (1H, dd, J 8.0, 1.0 Hz), 8.91 (1H, s), 9.48 (1H, d, J=2.2 Hz). The nitrite (4 mg) was taken up in a solution of ammonia in methanol (2 M, 0.75 ml). 2 drops of a slurry of 10% Pd/C in water were added and the reaction stirred under a balloon of hydrogen. After 1 h the product had been consumed and the reaction was filtered and the filtrate condensed in vacuo. The crude amine was taken up in toluene (1 ml) and 4-trifluoromethyl phenylisothiocyanate (4 mg) was added and the reaction stirred at room temperature for 2 h. A further isothiocyanate (1 mg) was added and the reaction stirred an additional 90 min. Dicyclohexylcarbodiimide (4 mg) was added and the reaction heated to 100° C. After 45 min the reaction was condensed and purified by gradient column chromatography, eluting with 3:1 to 1:1 hexane:ethyl acetate followed SCX column chromatography eluting with methanol then ammonia in methanol (2M) to give the desired imidazopyridizine (2.75 mg). MS: (ES (M+1)), 424 ¹H NMR (500 MHz, MeOH-d₄) 7.46 (1H, s), 7.57 (2H, d, J=8.6 Hz), 7.65 (1H, ddd, J 8.1, 4.9, 0.8 Hz), 7.76 (2H, d, J=8.5 Hz), 8.04 (1H, d, J=2.1 Hz), 8.27 (1H, d, J=2.2 Hz), 8.30 (1H, dd, J 8.1, 1.3 Hz), 8.90 (1H, s).

### EXAMPLE 76

### 5-Bromo-N-[4-trifluoromethylphenyl]-3-[3-trifluoromethylpyridin-2-yl]imidazo[1,5-b]pyridazin-7-amine

Example 75 (228 mg, 0.54 mmol) was dissolved in dichloromethane (3 ml) and a slurry of N-bromosuccinimide (96 mg, 0.54 mmol) in dichloromethane (2 ml) was added over 3 min at room temperature. The mixture was stirred for 5 min, then the solvent was evaporated and the residue purified by flash chromatography (eluant 50% EtOAc in isohexane) to give the title compound as a brown solid (203 mg). MS: (ES (M+1)) 502, 504. ¹H NMR (400 MHz, DMSO) δ 10.14 (1H, s), 8.99 (1H, d, J 4.0), 8.41 (1H, d, J 2.1), 8.39 (1H, dd, J 1.3 and 8.1), 7.96-7.88 (3H, m), 7.73 (1H, dd, J 4.7, 7.6), 7.66 (2H, d, J 8.7) ppm.

### EXAMPLE 77

### 5-(1-methyl-1H-imidazol-2-yl)-N-[4-trifluoromethylphenyl]-3-[3-trifluoromethylpyridin-2-yl]imidazo[1,5-b]pyridazin-7-amine

To 1-methylimidazole (47 μl, 0.59 mmol) in tetrahydrofuran (2 ml) at −78° C. was added n-butyllithium (385 μl, 0.616 mmol) in a dropwise fashion. After half an hour a solution of zinc chloride (244 mg, 1.79 mmol) in tetrahydrofuran (1 ml) was added via cannula. The reaction was allowed to warm to room temperature and stirred for a further hour. Tetrakis(triphenylphosphine)palladium(0) (5 mg, 0.004 mmol) and Example 76 (100 mg, 0.2 mmol) were added in tetrahydrofuran (1 ml) via cannula. The reaction mixture was then degassed and heated to reflux. After 16 h, the reaction was allowed to cool to room temperature, then poured into a solution of ethylenediaminetetraaceticacid disodium salt (5.5 g) in water (50 ml). The mixture was then basified by addition of solid Na₂CO₃ and extracted three times with ethyl acetate. After drying over sodium sulfate, the mixture was filtered and adsorbed onto silica gel. Purification by flash chromatography (50-80% ethyl acetate/iso-hexane) gave the title compound (18.1 mg, 18%) as a red solid, MS: (ES (M+1)) 504. ¹H NMR (360 MHz, DMSO) δ 10.12 (1H, s), 9.00 (1H, d, J=4.8 Hz), 8.67 (1H, d, J=2.2 Hz), 8.48 (1H, d, J=2.2 Hz), 8.39 (1H, d, J=8.1 Hz), 8.05 (2H, d, J=8.6 Hz), 7.71-7.67 (3H, m), 7.25 (1H, d, J=1.1 Hz), 7.02 (1H, d, J=1.1 Hz), 4.15 (3H, s) ppm.

### EXAMPLE 78

### N-(4-chlorophenyl)-3-[3-trifluoromethylpyridin-2-yl]imidazo[1,5-b]pyridazin-7-amine

Prepared from Description 10 and 4-chlorophenylisothiocyanate according to the procedure of Example 42, the title compound was obtained as a yellow-orange solid (8.5 mg).

m/z (ES⁺) 390, 392 (M+H⁺). ¹H NM (500 MHz, CDCl₃) 7.14 (1H, s), 7.31 (2H, d, J 9.0), 7.42 (1H, s), 7.47 (1H, m), 7.68 (2H, d, J 8.5), 7.90 (1H, d, J 4.4), 8.12 (1H, d, J 8.1), 8.21 (1H, d, J 4.6), 8.87 (1H, d, J 5.0) ppm.

### EXAMPLE 79

### 5-methyl-N-[4-trifluoromethylphenyl]-3-[3-trifluoromethylpyridin-2-yl]imidazo[1,5-b]pyridazin-7-amine

Prepared from Description 19 and 4-trifluoromethylphenylisothiocyanate according to the sequence described in Example 75, the title compound was obtained as a yellow-orange solid (8 mg). m/z (ES⁺) 438 M+H⁺). ¹H NMR (500 MHz, MeOH-d₄) 8.88 (1H, d, J 3.8), 8.29 (1H, dd, J 1.3, 8.1), 8.17 (1H, d, J 2.1), 7.98 (1H, d, J 2.0), 7.73 (2H, d, J 8.5), 7.63-7.61 (1H, m), 7.56 (2H, d, J 8.6), 2.51 (3H, s) ppm.

### EXAMPLE 80

### 7-{[4-Trifluoromethylphenyl]amino}-3-[3-trifluoromethylpyridin-2-yl]imidazo[1,5-b]pyridazine-5-carbonitrile

A mixture of Example 76 (38.5 mg, 0.077 mmol) zinc cyanide (5.4 mg) zinc metal (nanosize activated powder, 0.5 mg) and [1,1′-bis(diphenylphosphino)ferrocene]dichloropalladium(II) dichloromethane complex (3 mg) in N,N-dimethylacetamide (1 ml) was heated at 160° C. for 20 min in a microwave reactor. After cooling to room temperature, the mixture was partitioned by addition of water (10 ml), saturated aqueous NaHCO₃ (10 ml) and EtOAc (20 ml). The aqueous phase was extracted with EtOAc (10 ml) and the combined organic phases were washed with water (10 ml). The solvent was evaporated and the residue purified by preparative thin layer chromatography (eluant dichloromethane) to give the title compound (15 mg). MS: (ES (M+1)) 449. ¹H NMR (500 MHz, DMSO) δ 10.36 (1H, 9), 9.03 (1H, d, J 4), 8.68 (1H, d, J 2), 8.44 (1H, d, J 8), 8.40 (1H, d, J 2), 8.04 (2H, d, J 8.6), 7.80 (1H, dd, J 4, 8), 7.70 (2H, d, J 8.6) ppm.

### EXAMPLE 81

### 7-{[4-Trifluoromethylphenyl]amino}-3-[3-trifluoromethylpyridin-2-yl]imidazo[1,5-b]pyridazine-5-carboxamide

Concentrated hydrochloric acid (2 ml) was added to a sample of Example 80 (38 mg) and the mixture heated at 80° C. for 20 min. The mixture was cooled to room temperature, diluted with water (20 ml) and extracted with EtOAc (2×20 ml). The combined organic phases were evaporated. Trituration of the residue with diethyl ether (×2) led to a pure sample of the title compound (13 mg). MS: (ES (M+1)) 467. ¹H NMR (500 MHz, DMSO) δ 10.10 (1H, s), 9.01 (1H, d, J 4.5), 8.56 (1H, d, J 2), 8.51 (1H, d, J 2), 8.41 (1H, d, J 7.7), 8.17 (2H, d, J 8.6), 7.75 (1H, dd, J 4.5 and 7.7), 7.72 (1H, s), 7.62 (2H, d, J 8.6), 7.41 (1H, s).

### EXAMPLE 82

### 3-(3-Methylpyridin-2-yl)-N-[4-trifluoromethylphenyl]imidazo[1,5-b]-pyridazin-7-amine

A mixture of Description 21 (945 mg) and 10% palladium on carbon (250 mg) in 2M methanolic ammonia (45 μl) was stirred under a balloon of hydrogen for 5 h. The catalyst was removed by filtration and the solvent evaporated. Toluene (10 ml) was added, then removed in vacua. A further portion of toluene (10 ml) was then added to the residue and a solution of 4-trifluoromethylphenylisothiocyanate (1.03 g) in toluene (20 ml) was added. The mixture was stirred at 50° C. for 1 h. Dicyclohexylcarbodiimide (1.06 g) was then added and the mixture heated at 100° C. for 3 h. After cooling to room temperature, flash silica (ca. 20 ml) was added and the solvent evaporated. Purification by flash column chromatography (eluant 15% EtOAc in isohexane gradually increasing to 75% EtOAc in isohexane) gave the title compound (1.03 g) as an orange solid. MS: (ES (M+1)) 370. ¹H NMR (500 MHz, DMSO) δ 9.87 (1H, s), 8.54 (1H, dd, J=1.2, 4.7 Hz), 8.50 (1H, d, J=2.1 Hz), 8.26 (1H, d, J=2.1 Hz), 7.97 (2H, d, J=8.6 Hz), 7.78 (1H, br. d, J=7 Hz), 7.62 (2H, d, J=8.6 Hz), 7.44 (1H, s), 7.34 (1H, dd, J=4.7, 7.7 Hz) ppm.

### EXAMPLE 83

### 3-(3-Methylpyridin-2-yl)-7-{[4-trifluoromethylphenyl]amino}imidazo[1,5-b]pyridazine-5-carbonitrile

Prepared from Example 82 according to the procedures of Examples 76 and 80 respectively. MS: (ES (M+1)) 395. ¹H NMR (500 MHz, DMSO) a 10.33 (1H, s), 8.75 (1H, d, J=1.9 Hz), 8.59 (1H, br. d, J=4.1 Hz), 8.46 (1H, d, J=1.9 Hz), 8.04 (2H, d, J=8.6 Hz), 7.84 (1H, br. d, J=7.9 Hz), 7.69 (2H, d, J=8.6 Hz), 7.42 (1H, dd, J=4.7, 7.6 Hz), 2.53 (3H, s) ppm.

### EXAMPLE 84

### 5-Phenyl-N-[4-trifluoromethylphenyl]-3-[3-trifluoromethylpyridin-2-yl]imidazo[1,5-b]pyridazin-7-amine

A mixture of Example 76 (30 mg, 0.06 mmol), phenylboronic acid (8.2 mg, 0.067 mmol), saturated aqueous Na₂CO₃ solution (70 μl, 0.12 mmol) and [1,1′-bis(diphenylphosphino)ferrocene]dichloropalladium(II) dichloromethane complex (3 mg) in dioxane (1 ml) was heated at 150° C. for 35 min in a microwave reactor. More phenylboronic acid (2 mg, 0.016 mmol), catalyst (3 mg) and saturated aqueous Na₂CO₃ solution (2 drops) were added and the mixture heated at 160° C. for 15 min in the microwave reactor. After cooling to room temperature, the mixture was partitioned between EtOAc (10 ml) and water (10 ml). The layers were separated, the aqueous phase extracted with more EtOAc (2×10 ml) and the combined organic layers were evaporated. The residue was purified by preparative thin layer chromatography (eluant CH₂Cl₂) to give the title compound (10 mg).

MS: (ES (M+1)) 500. ¹H NMR (400 MHz, DMSO) δ 10.03 (1H, s), 9.00 (1H, d, J=4.7 Hz), 8.46 (1H, d, J=2.0 Hz), 8.41-8.39 (2H, m), 8.13 (2H, d, J=8.6 Hz), 7.97 (2H, d, J=8 Hz), 7.75-7.71 (1H, m), 7.68 (2H, d, J=8.6 Hz), 7.48 (2H, app. t, J=8 Hz), 7.33 (1H, app. t, J=8 Hz) ppm.

### EXAMPLE 85

### 5-Pyridin-4-yl-N-[4-trifluoromethylphenyl]-3-[3-trifluoromethylpyridin-2-yl]imidazo[1,5-b]pyridazin-7-amine

Prepared from Example 76 and 4-pyridylboronic acid according to the procedure of Example 84.

MS: (ES (M+1)) 501. ¹H NMR (500 MHz, DMSO) δ 10.12 (1H, s), 9.02 (1H, d, J=3.9 Hz), 8.66 (1H, d, J=1.8 Hz), 8.62-8.59 (2H, m), 8.50 (1H, d, J=1.8 Hz), 8.43 (1H, dd, J=1.1, 8.0 Hz), 8.16 (2H, d, J=8.6 Hz), 7.96-7.92 (2H, m), 7.77 (1H, dd, J=5.1, 8.0 Hz), 7.70 (2H, d, J=8.7 Hz) ppm.

### EXAMPLE 86

### 5-Pyridin-3-yl-N-[4-trifluoromethylphenyl]-3-[3-trifluoromethylpyridin-2-yl]imidazo[1,5-b]pyridazin-7-amine

Prepared from Example 76 and 3-pyridylboronic acid according to the procedure of Example 84.

MS: (ES (M+1)) 501. ¹H NMR (500 MHz, DMSO) δ 10.09 (1H, s), 9.18 (1H, s), 9.01 (1H, d, J=4.4 Hz), 8.55 (1H, s), 8.51 (1H, br. s), 8.45 (1H, s), 8.41 (1H, d, J=8.1 Hz), 8.33 (1H, d, J=7.8 Hz), 8.14 (2H, d, J=8.5 Hz), 7.74 (1H, dd, J=4.7, 7.6 Hz), 7.69 (2H, d, J=8.5 Hz), 7.49 (1H, dd, J=4.7, 7.6 Hz) ppm.

### EXAMPLE 87

### 5-(Morpholin-4-ylmethyl)-N-[4-trifluoromethylphenyl]-3-[3-trifluoromethyl pyridin-2-yl]imidazo[1,5-b]pyridazin-7-amine

A mixture of Example 75 (56 mg, 0.13 mmol), morpholine (12 mg, 0.14 mmol) formaldehyde (37% aq. solution, 13 μl), dichloromethane (1.5 ml) and aqueous acetic acid (1 mmol/ml, 0.13 ml) was stirred at room temperature for 24 h. The mixture was partitioned between EtOAc (15 ml) and 1N aq. NaOH (10 ml). The organic phase was evaporated, then the residue purified by flash column chromatography (eluant 5% MeOH in CH₂Cl₂). Further purification was effected by loading the residue onto an acidic ion-exchange cartridge, washing away non-basic impurities with methanol, then eluting with 2M methanolic ammonia to give the title compound (50 mg) as an orange solid. MS: (ES (M−R₂N)) 436. ¹H NMR (500 MHz, CDCl₃) δ 8.88 (1H, dd, J=1.0, 4.7 Hz), 8.22 (1H, d, J=2.0 Hz), 8.15 (1H, d, J=2.0 Hz), 8.13 (1H, dd, J=1.3, 8 Hz), 7.81 (2H, d, J=8.6 Hz), 7.60 (2H, d, J=8.6 Hz), 7.47 (1H, dd, J=4.7, 8 Hz), 7.30 (1H, s), 3.87 (2H, s), 3.73 (4H, t, J=4.6 Hz), 2.59 (4H, br. t, J=4.6 Hz) ppm.

### EXAMPLE 88

### 5-[Dimethylaminomethyl]-N-[4-trifluoromethylphenyl]-3-[3-trifluoromethyl pyridin-2-yl]imidazo[1,5-b]pyridazin-7-amine

A mixture of Example 75 (90 mg, 0.213 mmol), dimethylamine (40% aq. solution, 100 μl), formaldehyde (37% aq. solution, 88 μl), dichloromethane (2 ml) and aqueous acetic acid (1 mmol/ml, 0.21 ml) was stirred at room temperature for 20 h, then at 50° C. for 4 h. The mixture was cooled to room temperature then partitioned between EtOAc (15 ml) and 1N aq. NaOH (10 ml). The organic phase was evaporated, then the residue purified by flash column chromatography (eluant 5% MeOH in CH₂Cl₂) to give the title compound (69 mg) as an orange solid. MS: (ES (M−R₂N)) 436. ¹H NMR (500 MHz, CDCl₃) δ 8.87 (1H, dd, J=1.0, 4.7 Hz), 8.21 (1H, d, J=2.0 Hz), 8.12 (1H, dd, J=1.4, 8 Hz), 8.10 (1H, d, J=2.0 Hz), 7.83 (2H, d, J=8.6 Hz), 7.59 (2H, d, J=8.6 Hz), 7.46 (1H, dd, J=4.7, 8 Hz), 7.30 (1H, s), 3.80 (2H, s), 2.35 (6H, s) ppm.

### EXAMPLE 89

### 5-(1H-Imidazol-1-ylmethyl)-N-[4-trifluoromethylphenyl]-3-[3-trifluoromethyl pyridin-2-yl]imidazo[1,5-b]pyridazin-7-amine

A mixture of Example 88 (69 mg, 0.146 mmol), imidazole (12 mg, 0.175 mmol) and iodomethane (15 μl, 0.24 mmol) in xylene (3 ml) was stirred at room temperature for 1 h, then at 100° C. for 20 min. The precipitated solid (32 mg) was collected by filtration and analysis showed this to be the quaternary salt from reaction of Example 88 with iodomethane. The solid was re-suspended in xylene (3 ml), more imidazole (12 mg, 0.175 ml) was added and the mixture heated at 130° C. for 3 h after which time the solid had all dissolved. Evaporation and purification of the residue by preparative thin layer chromatography (eluant 5% MeOH in CH₂Cl₂) gave the title compound (25 mg) as an orange solid. MS: (ES (M−R₂N)) 436. ¹H NMR (400 MHz, CDCl₃) δ 8.86 (1H, dd, J=1.0, 4.7 Hz), 8.19 (1H, d, J=2.1 Hz), 8.12 (1H, dd, J=1.4, 8.0 Hz), 7.82 (2H, d, J=8.6 Hz), 7.67 (1H, s), 7.62 (2H, d, J=8.6 Hz), 7.51-7.47 (1H, m), 7.41 (1H, br.s), 7.35 (1H, s), 7.08 (2H, s), 5.41 (2H, s) ppm.

### EXAMPLE 90

### 7-{[4-Trifluoromethylphenyl]amino}-3-[3-trifluoromethylpyridin-2-yl]imidazo[1,5-b]pyridazine-5-carboxylic acid

A mixture of Example 76 (205 mg, 0.41 mmol) and sodium acetate (67 mg, 0.82 mmol) in ethanol (5 ml) was degassed (by bubbling through N₂). [1,1′-bis(diphenyl-phosphino)ferrocene]dichloropalladium(II) dichloromethane complex (15 mg) was added, then CO gas was bubbled through the mixture at a vigorous rate for 5 min. The flow of CO was reduced to a gentle flow and the mixture was heated to reflux for 3 h. The mixture was then cooled to room temperature and the ethanol evaporated. The residue was then partitioned between EtOAc (15 ml) and saturated aq. NaHCO₃ (15 ml). The organic phase was evaporated and purified by flash column chromatography (eluant 5% MeOH in CH₂Cl₂) to give the corresponding ethyl ester (210 mg, >theory). A sample of the ester (69 mg, 0.143 mmol) was dissolved in a mixture of methanol (3 ml), water (1 ml) and THF (1 ml). Lithium hydroxide monohydrate (6 mg, 0.143 mmol) was added and the mixture stirred at room temperature for 24 h. The solvents were evaporated, then 1N HCl (10 ml) was added and the product extracted with EtOAc (2×10 ml). The organic phase was evaporated and the residue purified by flash column chromatography eluting with 1% AcOH in dichloromethane to give the title compound (20 mg). MS: (ES (M+1)) 468. ¹H NMR (500 MHz, DMSO) δ 12.8 (1H, br. s), 10.13 (1H, s), 9.02 (1H, d, J=4.3 Hz), 8.62 (1H, s), 8.47 (1H, s), 8.42 (1H, d, J=7.5 Hz), 8.10 (2H, d, J=8.6 Hz), 7.77 (1H, dd, J=4.8, 7.8 Hz), 7.68 (2H, d, J=8.6 Hz) ppm.

### EXAMPLE 91

### N-[4-Trifluoromethylphenyl]-7-[3-trifluoromethylpyridin-2-yl]imidazo[1,2-b]pyridazin-3-amine

A mixture of Description 14 (150 mg, 0.55 mmol), 4-bromobenzotrifluoride (125 mg, 77 μl, 0.55 mmol) and caesium carbonate (254 mg, 0.78 mmol) in 1,4-dioxan (5 ml) was degassed (N₂×3), then 9,9-dimethyl-4,5-bis(diphenylphosphino)xanthene [xantphos] (19.3 mg, 0.033 mmol) and tris(dibenzylideneacetone)dipalladium (0) (10.2 mg, 0.011 mmol) were added and the mixture degassed again (N₂×3). The reaction was then heated to 100° C. for 24 h under nitrogen, then cooled to room temperature and diluted with tetrahydrofuran (20 ml). The mixture was then filtered through a glass fibre pad and the filtrate evaporated. The residue was purified by flash column chromatography (eluant 1:39 MeOH—CH₂Cl₂) and also by mass-directed preparative hplc to give the title compound as a yellow-orange solid (115 mg). ¹H NMR (500 MHz, DMSO) δ 9.06 (1H, s), 9.02 (1H, d, J=5 Hz), 8.70 (1H, d, J=1.5 Hz), 8.43 (1H, d, J=8 Hz), 8.24 (1H, d, J 1.5 Hz), 7.94 (1H, s), 7.78 (1H, dd, J 8, 5 Hz), 7.53 (2H, d, J=8 Hz), 6.96 (2H, d, J=8 Hz); MS: (ES (M+1)), 424.

### EXAMPLE 92

### 7-[3-Trifluoromethylpyridin-2-yl]-N-[5-trifluoromethylpyridin-2-yl]imidazo[1,2-b]pyridazin-3-amine

Prepared from Description 14 and 2-bromo-5-trifluoromethylpyridine according to the procedure of Example 91, the title compound was obtained as a yellow-orange solid (45 mg).

¹H NMR (500 MHz, DMSO) δ 10.20 (1H, s), 9.01 (1H, br. d, J=5 Hz), 8.72 (1H, d, J=2 Hz), 8.54 (1H, br. s), 8.42 (1H, dd, J 8, 1.5 Hz), 8.22 (1H, d, J=2 Hz), 8.21 (1H, s), 7.94 (1H, dd, J 9, 2 Hz), 7.76 (1H, dd, J 8, 5 Hz), 7.20 (1H, d, J=9 Hz); MS: (ES I+1)), 425.

### EXAMPLE 93

### N-(5-Methylpyridin-2-yl)-7-[3-trifluoromethylpyridin-2-yl]imidazo[1,2-b]pyridazin-3-amine

Prepared from Description 14 and 2-bromo-5-methylpyridine according to the procedure of Example 91, the title compound was obtained as a yellow-orange solid (5 mg).

¹H NMR (500 MHz, DMSO) δ 9.48 (1H, s), 9.01 (1H, d, J=5 Hz), 8.67 (1H, d, J=2 Hz), 8.41 (1H, br. d, J=8 Hz), 8.21 (1H, s), 8.15 (1H, d, J=2 Hz), 8.04 (1H, d, J=2 Hz), 7.75 (1H, dd, J 8, 5 Hz), 7.47 (1H, dd, J 8, 2 Hz), 7.05 (1H, d, J=8 Hz), 2.20 (3H, s); MS: (ES (M+1)), 371.

### EXAMPLE 94

### 7-[1-Oxido-3-trifluoromethylpyridin-2-yl]-N-[4-trifluoromethylphenyl]-imidazo[1,2-b]pyridazin-3-amine

Example 91 (64 mg, 0.15 mmol) was dissolved in chloroform (5 ml) and OXONE® (100 mg) and wet alumina (150 mg) [10 g water per 50 g alumina] were added. The mixture was heated at reflux for 18 h. Extra OXONE® (100 mg) and wet alumina (150 mg) were then added and the reaction heated for a further 1.5 h, then left to stand at room temperature for 4 days. The mixture was filtered, the solvent evaporated and the residue purified by preparative thin layer chromatography to give the title compound (4 mg). MS: (ES (M+1)) 440. ¹H NMR (500 MHz, DMSO) δ 9.04 (s, 1H), 8.69 (d, 1H, J=6.5 Hz), 8.60 (d, 1H, J=1.8 Hz), 8.27 (br. s, 1H), 7.91 (s, 1H), 7.89 (d, 1H, J=8.2 Hz), 7.76 (app. t, 1H, J=7.4 Hz), 7.53 (d, 2H, J=8.5 Hz), 6.99 (d, 2H, J=8.5 Hz) ppm.

### EXAMPLE 95

### 2-Bromo-N-[4-trifluoromethylphenyl]-7-[3-trifluoromethylpyridin-2-yl]imidazo[1,2-b]pyridazin-3-amine

Example 91 (513 mg, 1.21 mmol) was dissolved in acetic acid (4 ml), a solution of bromine in acetic acid (10% w/v, 2.4 ml, 1.5 mmol) was added and the mixture warmed to 100° C. After 5 min at this temperature more bromine solution (1.2 ml) was added, then 10 min later a further 1.2 ml of the bromine solution was added. After stirring for 10 min more, the reaction was cooled to room temperature and the acetic acid and excess bromine evaporated. The residue was partitioned between saturated aqueous sodium bicarbonate (25 ml) and ethyl acetate (25 ml) and the organic layer evaporated. Purification by flash chromatography (eluant 2.5% MeOH in CH₂Cl₂), then a second purification by flash chromatography (eluant 25% EtOAc in CH₂Cl₂) gave the title compound (237 mg), MS: (ES (M+1)) 502, 504. ¹H NMR (400 MHz, DMSO) δ 9.01 (dd, 1H, J=1, 4.7 Hz), 8.94 (s, 1H), 8.74 (d, 1H, J=2 Hz), 8.43 (dd, 1H, J=1, 8 Hz), 8.28 (d, 1H, J=2 Hz), 7.79-7.77 (m, 1H), 7.49 (d, 2H, J=8.5 Hz), 6.73 (d, 2H, J=8.5 Hz) ppm.

### EXAMPLE 96

### 3-{[4-Trifluoromethylphenyl]amino}-7-[3-trifluoromethylpyridin-2-yl]imidazo[1,2-b]pyridazine-2-carbonitrile

Prepared from Example 95 according to the method of Example 80. MS: (ES (M+1)) 449. ¹H NMR (400 MHz, DMSO) δ 9.67 (1H, s), 9.02 (1H, d, J 4.0), 8.85 (1H, d, J 2), 8.44 (1H, dd, J 1.2 and 8.0), 8.35 (1H, d, J 2), 7.83-7.77 (1H, m), 7.58 (2H, d, J 8.6), 7.03 (2H, d, J 8.6) ppm.

### EXAMPLE 97

### 2-Methyl-N-[4-trifluoromethylphenyl]-7-[3-trifluoromethylpyridin-2-yl]imidazo[1,2-b]pyridazin-3-amine

Prepared from Description 22 according to the procedures of Descriptions 13, 14 and Example 91 respectively. MS: (ES (M+1)) 438. ¹H NMR (500 MHz, DMSO) δ 8.99 (1H, d, J=4.7 Hz), 8.74 (1H, s), 8.61 (1H, d, J=2 Hz), 8.41 (1H, dd, J=1.2, 8 Hz), 8.14 (1H, d, J=2 Hz), 7.76 (1H, dd, J=4.7, 8 Hz), 7.47 (2H, d, J=8.5 Hz), 6.66 (2H, d, J=8.5 Hz), 2.36 (3H, s).

### EXAMPLE 98

### 7-[3-Trifluoromethylpyridin-2-yl]-N-[6-trifluoromethylpyridin-3-yl]imidazo[. 2-b]Pyridazin-3-amine hydrochloride

A mixture of Description 14 (200 mg, 0.7 mmol), 5-bromo-2-trifluoromethylpyridine (160 mg, 0.7 mmol), caesium carbonate (360 mg, 1 mmol), 9,9-dimethyl-4,5-bis(diphenylphosphino)xanthene [xantphos] (25 mg, 0.043 mmol) and tris(dibenzylideneacetone)dipalladium (0) (13 mg, 0.014 mmol) in 1,4-dioxane (6 ml) was degassed (N₂×3) and then heated at 110° C. for 16 h under nitrogen. The cooled reaction mixture was diluted with tetrahydrofuran (20 ml) and filtered through hyflo, flushing through with THF. The filtrate was evaporated and the residue was purified by flash column chromatography over silica using an eluant system of 1:1 EtOAc:DCM to 100% EtOAc. The hydrochloride salt was then formed according to Example 119 (0.1 g, 34%).

¹H NMR (500 MHz, DMSO) δ 9.45 (1H, s), 9.03 (1H, d, J 3.9), 8.80 (1H, d, J 2.0), 8.45 (1H, d, J 8.1), 8.38 (1H, d, J 2.7), 8.34 (1H, d, J 1.8), 8.08 (1H, s), 7.80 (1H, dd, J 4.8 and 8.1) 7.69 (1H, d, J 8.6), 7.32 (1H, dd, J 2.7 and 8.4) ppm. m/z (ES⁺) 425 (M+H⁺)

Examples 99-103 were prepared from Description 14 and the indicated compound using the procedure of Example 98.

### EXAMPLE 99

### N-(4-Chlorophenyl)-7-[3-trifluoromethylpyridin-2-yl]imidazo[1,2-b]pyridazin-3-amine hydrochloride

1-Chloro-4-iodobenzene gave (32 mg, 4.6%).

¹H NMR (500 MHz, DMSO) δ 9.02 (1H, d, J=5.1 Hz), 8.75 (1H, s), 8.72 (1H, d, J=2.0 Hz), 8.43 (1H, d, J=9.4 Hz), 8.23 (1H, s), 7.90 (1H, s), 7.78 (1H, dd, J=4.6, 7.4 Hz), 7.25 (2H, d, J=8.8 Hz), 6.93 (2H, d, J=8.8 Hz) ppm. m/z (ES⁺) 390 (M+H⁺)

### EXAMPLE 100

### N-[2-Fluoro-4-trifluoromethylphenyl]-7-[3-trifluoromethylpyridin-2-yl]imidazo[1,2-b]pyridazin-3-amine hydrochloride

1-Bromo-2-fluoro-4-trifluoromethylbenzene gave (0.2 g, 42%).

¹H NMR (500 MHz, DMSO) δ 9.01 (1H, d, J 4.1), 8.84 (1H, s), 8.74 (1H, d, J 1.9), 8.43 (1H, d, J 6.9), 8.32 (1H, d, J 1.8), 7.98 (1H, s), 7.78 (1H, dd, J 5.1 and 8.1), 7.62 (1H, d, J 11.6), 7.31 (1H, d, J 8.8), 6.61 (1H, t, J 8.5) ppm. m/z (ES⁺) 442 (M+H⁺)

### EXAMPLE 101

### N-(6-Methylpyridin-3-yl)-7-[3-trifluoromethylpyridin-2-yl]imidazo[1,2-b]pyridazin-3-amine hydrochloride

5-Bromo-2-methylpyridine gave (18 mg, 4.5%).

¹H NMR (500 MHz, DMSO) δ 9.66 (1H, 8), 9.01 (1H, d, J 4.1), 8.76 (1H, d, J 1.9), 8.43 (1H, d, J 6.8), 8.33 (1H, d, J 1.7), 8.17 (1H, d, J 2.6), 8.06 (1H, s), 7.91 (1H, dd, J 2.7 and 8.9), 7.79 (1H, dd, J 4.3 and 7.6), 7.72 (1H, d, J 8.8), 2.61 (3H, s) ppm.

m/z (ES⁺) 371 (M+H⁺)

### EXAMPLE 102

### N-[4-Trifluoromethoxyphenyl]-7-[3-trifluoromethylpyridin-2-yl]imidazo[1,2-b]pyridazin-3-amine hydrochloride

1-Bromo-4-(trifluoromethoxybenzene gave (62 mg, 12%).

¹H NMR (500 MHz, DMSO) δ 9.28 (1H, d, J 4.2), 9.16 (1H, s), 9.08 (1H, s), 8.69 (1H, d, J 6.8), 8.58 (1H, s), 8.28 (1H, s), 8.04 (1H, dd, J 5.2 and 8.1), 7.47 (2H, d, J 8.6), 7.26 (2H, t, J 4.5) ppm.

m/z (ES⁺) 440 (M+H⁺)

### EXAMPLE 103

### N-[3-Fluoro-4-trifluoromethylphenyl]-7-[3-trifluoromethyl-pyridin-2-yl]imidazo[1,2-b]pyridazin-3-amine hydrochloride

4-Bromo-2-fluoro-1-trifluoromethylbenzene gave (0.33 g, 64%).

¹H NMR (500 MHz, DMSO) δ 9.42 (1H, s), 9.03 (1H, d, J 4.4), 8.82 (1H, s), 8.45 (1H, d, J 8.2), 8.35 (1H, s), 8.09 (1H, s), 7.80 (1H, dd, J 4.8 and 7.9), 7.56 (1H, t, J 8.7), 6.82 (2H, t, J 7.7) ppm.

m/z (ES⁺) 442 (h+H⁺)

### EXAMPLE 104

### 7-(3-Chloropyridin-2-yl)-N-[4-trifluoromethylphenyl]imidazo[1,2-b]pyridazin-3-amine hydrochloride

Prepared from Description 24 and 4-1-trifluoromethylbenzene according to the procedure of Example 91 and 119, the title compound was obtained as a yellow-orange solid (40 mg). ¹H NMR (500 MHz, CDCl₃) δ 8.86 (1H, d, J 1.9), 8.66 (in, dd, J 4.5, 1.5), 8.50 (1H, s), 7.86 (1H, dd, J 8.1, 1.4), 7.84 (1H, s), 7.55 (2H, d, J 8.5), 7.32 (1H, m), 7.07 (2H, d, J 8.6), 6.54 (1H, s) ppm; m/z (ES⁺) 390, 392 (M+H⁺).

### EXAMPLE 105

### N-(4-Chlorophenyl)-7-(3-chloropyridin-2-yl)imidazo[1,2-b]pyridazin-3-amine hydrochloride

Prepared from Description 24 and 1-chloro-4-iodobenzene according to the procedures of Example 91 and 119, the title compound was obtained as a yellow-orange solid (0.23 g). ¹H NMR (400 MHz, CDCl₃) 8.84 (1H, d, J 1.9), 8.65 (1H, d, J 4.1), 8.47 (1H, d, J 2.2), 7.86 (1H, d, J 8.2), 7.76 (1H, s), 7.30 (3H, m), 7.01 (2H, d, J 8.7), 6.40 (1H, s) ppm. m/z(ES⁺) 356, 358 (M+H⁺).

### EXAMPLE 106

### [7-(3-Methylpyridin-2-yl)[1,2,4]triazolo[4,3-b][1,2,4]triazin-3-yl]-(4-trifluoromethylphenyl)amine

To Description 17 (88 mg, 0.435 mmol) in acetonitrile was added 4-trifluoromethylphenylisocyanate (89 mg, 0.479 mmol). The white slurry was stirred for 72 h. Mass spectrometry showed (MH⁺) 390. Phosphorous oxychloride (81 μl, 0.874 mmol) was then added and the mixture heated to 90° C. After 4 h the red solution was allowed to cool to room temperature, then quenched by addition of saturated NaHCO₃. The mixture was extracted three times with ethyl acetate and dried over sodium sulfate. After filtration the compound was adsorbed onto silica gel and purified by flash column (50% ethyl acetate in hexanes) to yield the title compound (81.3 mg, 50%) as an orange solid.

¹H NMR (400 MHz, DMSO) δ 10.4 (1H, s) 9.26 (1H, s), 8.69 (1H, dd J 4.4, 0.8 Hz), 8.04 (2H, d, J=8.8 Hz), 7.94-7.91 (1H, m), 7.70 (2H, d, J=8.8 Hz), 7.56 (1H, dd, J 7.6, 4.8 Hz), 2.75 (3H, s) ppm; MS: (ES (M+1)), 372.

### EXAMPLE 107

### [7-(3-Trifluoromethyl-pyridin-2-yl)-[1,2,4]triazolo[4,3-b][1,2,4]triazin-3-yl]-(4-trifluoromethyl-phenyl)-amine

To Description 29 (63 mg, 0.246 mmol) in acetonitrile (5 ml) was added 4-trifluoromethylphenylisocyanate (60 mg, 0.319 mmol). The white slurry was stirred for 72 h. Mass spectrometry showed (MH⁺) 444. Phosphorus oxychloride (46 μl, 0.492 mmol) was then added and the mixture heated to 90° C. After 72 h the red solution was allowed to cool to room temperature, then quenched by addition of saturated NaHCO₃. The mixture was extracted three times with ethyl acetate and dried over sodium sulfate. After filtration the compound was adsorbed onto silica gel and purified by flash column (50% ethyl acetate in hexanes) to yield the title compound (40 mg, 38%) as an orange solid. MS: (ES (M+1)) 426.

¹H NMR (400 MHz, CDCl₃) δ 8.96-8.94 (2H, m) 8.28 (1H, dd J 8.4, 1.6), 7.93 (2H, d, J 8.4), 7.69-7.65 (3H, m), 7.19 (1H, m) ppm.

Examples 108-111 were obtained using a procedure analogous to Example 107 using the compound indicated.

### EXAMPLE 108

### N-(4-Chlorophenyl)-7-[3-trifluoromethylpyridin-2-yl][1,2,4]triazolo[4,3-b][1,2,4]triazin-3-amine

4-Chlorophenylisocyanate gave the title compound was obtained (40 mg, 35%) as an orange solid, MS: (ES (M+1)) 392.

¹H NMR (400 MHz, DMSO) δ 10.17 (1H, s), 9.13 (1H, s), 9.09 (1H, t, J=2.4 Hz), 8.53 (1H, dd, J=1.2, 8.1 Hz), 7.93-7.89 (3H, m), 7.43-7.39 (2H, m) ppm.

### EXAMPLE 109

### 4-({7-[3-Trifluoromethylpyridin-2-yl][1,2,4]triazolo[4,3-b][1,2,4]triazin-3-yl}amino)benzonitrile

4-Cyanophenylisocyanate gave the title compound (8 mg, 7%) as a red solid, MS: (ES (M+1)) 383.

¹H NMR (400 MHz, DMSO) δ 10.64 (1H, s), 9.16 (1H, s), 9.10 (1H, d, J=4.6 Hz), 8.54 (1H, d, J=6.9 Hz), 8.01 (2H, d, J=8.7 Hz), 7.91 (1H, dd, J=5.2, 8.3 Hz), 7.79 (2H, d, J=8.7 Hz) ppm.

### EXAMPLE 110

### 7-(3-Chloropyridin-2-yl)-N-[4-trifluoromethylphenyl][1,2,4]triazolo[4,3-b][1,2,4]triazin-3-amine

Description 30 gave the title compound (43 mg, 34%) as a red solid, MS: (ES (M+1)) 392.

¹H NMR (400 MHz, DMSO) δ 10.46 (1H, s), 9.16 (1H, s), 8.81 (1H, dd, J=1.4, 4.6 Hz), 8.24 (1H, dd, J=1.5, 8.2 Hz), 8.05 (2H, d, J=8.6 Hz), 7.72-7.68 (3H, m) ppm.

### EXAMPLE 111

### N-(4-Chlorophenyl)-7-(3-chloropyridin-2-yl)[1,2,4]triazolo[4,3-b][1,2,4]triazin-3-amine

Description 30 and 4-chlorophenylisocyanate gave the title compound (9 mg, 8%) as an orange solid, MS: (ES (M+1)) 359.

¹H NMR (400 MHz, DMSO) δ 10.14 (1H, s), 9.13 (1H, s), 8.80 (1H, dd, J=1.2, 4.4 Hz), 8.23 (1H, dd, J=1.3, 8.2 Hz), 7.91 (2H, d, J=9.0 Hz), 7.74-7.68 (1H, m), 7.40 (2H, d, J=9.0 Hz) ppm.

### EXAMPLE 112

### 3-(3-Methylpyridin-2-yl)-N-[4-trifluoromethylphenyl]imidazo[1,2-b][1,2,4]triazin-7-amine

Prepared from Description 32 according to the procedures of Descriptions 13, 14 and Example 91 respectively. MS: (ES (M+1)) 371. ¹H NMR (360 MHz, DMSO) δ 9.27 (1H, s), 9.18 (1H, s), 8.63 (1H, d, J=4.0 Hz), 8.03 (1H, s), 7.88 (1H, d, J=7.7 Hz), 7.53-7.47 (3H, m), 7.00 (2H, d, J=8.4 Hz), 2.74 (3H, s).

### EXAMPLE 113

### 3-(3-Chloropyridin-2-yl)-N-[4-trifluoromethylphenyl]imidazo[1,2-b][1,2,4]triazin-7-amine hydrochloride

Prepared from Description 36 and 1-bromo-4-trifluoromethylbenzene according to the procedures of Examples 98 and 119 (37 mg, 29%).

¹H NMR (500 MHz, DMSO) δ 9.27 (1H, s), 9.17 (1H, s), 8.77 (1H, dd, J 1.3 and 4.5), 8.21 (1H, dd, J 1.3 and 8.2), 8.14 (1H, s), 7.64 (1H, dd, J 4.5 and 8.2), 7.54 (2H, d, J 8.7), 7.05 (2H, d, J 8.6) ppm. m/z (ES⁺) 391 (M+H⁺).

### EXAMPLE 114

### N-[4-Trifluoromethylphenyl]-3-[3-trifluoromethylpyridin-2-yl]imidazo[1,2-b][1,2,4]triazin-7-amine

Description 40 (104 mg, 0.371 mmol), 4-bromobenzotrifluoride (52 μl, 0.371 mmol), caesium carbonate (171 mg, 0.524 mmol), XANTPHOS (12 mg) and tris(dibenzylideneacetone)dipalladium(0)-chloroform adduct (7 mg, 0.0067 mmol) were dissolved in dioxane (4 ml) and degassed. The reaction mixture was then heated to 100° C. for 16 h, then allowed to cool and filtered through celite (washing with ethyl acetate) and preabsorbed onto silica gel. Purification by column chromatography gave the title compound (17 mg, 11%) as a red solid.

MS: (ES (M+1)) 425. ¹H NMR (400 MHz, DMSO) δ 9.22 (1H, s), 9.10 (1H, s), 8.97 (1H, d, J=3.6 Hz), 8.41 (1H, dd, J=1.3, 8.1 Hz), 8.08 (1H, s), 7.76 (1H, dd, J=4.6, 8.2 Hz), 7.46 (2H, d, J=8.6 Hz), 6.98 (2H, d, J=8.6 Hz) ppm.

### EXAMPLE 115

### [7-(1-Methyl-1H-imidazol-2-yl)[1,2,4]triazolo[4,3-b]pyridazin-3-yl]-(4-trifluoromethylphenyl)amine

### a) 5-(1-Methyl-1H-imidazol-2-v)-2-(tetrahydro-pyran-2-yl)-2H-pyridazin-3-one

To 1-methylimidazole (5.5 ml, 69.6 mmol) in tetrahydrofuran (110 ml) at −78° C. was n-butyllithium (47.85 ml, 76.56 mmol) in a dropwise fashion. After half an hour a solution of zinc chloride (28 g, 0.20 mol) in tetrahydrofuran (165 ml) was added via cannula. The reaction was allowed to warm to room temperature and stirred for a further two hours. Tetrakis(triphenylphosphine)palladium(0) (1.3 g, 1.12 mmol) and 5-chloro-2-(tetrahydropyran-2-yl)-2H-pyridazin-3-one (5 g, 23.2 mmol) were added in tetrahydrofuran (110 ml) via cannula. The reaction mixture was then degassed and heated to reflux. After 16 h, the reaction was allowed to cool to room temperature, then the reaction mixture poured into a solution of ethylenediaminetetraaceticacid disodium salt (70 g) in water (600 ml). The mixture was then basified by addition of solid Na₂CO₃ and extracted three times with ethyl acetate. After drying over sodium sulfate, the mixture was filtered and concentrated in vacuo to give a yellow solid. Triturated with ethyl acetate afforded the title compound as a pale yellow solid (3.5 g, 58%).

¹H NMR (400 MHz, DMSO) δ 8.39 (1H, d, J=2.2 Hz), 7.45 (1H, s), 7.17 (1H, d, J=2.2 Hz), 7.15 (1H, s) 5.90 (1H, dd J 10.8, 1.8 Hz) 4.04-3.96 (1H, m), 3.89 (3H, s), 3.65-3.58 (1H, m) 2.16-2.06 (1H, m), 1.99-1.94 (1H, m), 1.72-1.63 (2H, m), 1.54-1.48 (2H, m) ppm; MS: (ES (M+1)), 261.

### b) 3-Chloro-5-(1-methyl-1H-imidazol-2-yl)pyridazine

To the product of step a) (1 g, 3.84 mmol) was added phosphorous oxychloride (10 ml) and the reaction heated to 90° C. After 10 min the reaction was allowed to cool and concentrated in vacuo. The orange residue was poured into ice/water and this mixture basified by addition of solid Na₂CO₃. The mixture was extracted with chloroform, dried over sodium sulfate, filtered and concentrated in vacuo to give the crude title compound (0.8 g).

¹H NMR (400 MHz, DMSO) δ 9.54 (1H, d, J=1.8 Hz), 7.84 (1H, d, J=1.8 Hz), 7.27 (1H, s), 7.12 (1H, s), 3.95 (3H, s) ppm; MS: (ES (M+1)), 195.

c) [5-(1-Methyl-1H-imidazol-2-yl)-pyridazin-3-yl]hydrazine

To the crude product of step b) (assuming 3.84 mmol) in isopropanol (10 ml) was added hydrazine monohydrate (0.14 ml, 19.2 mmol) and the reaction heated to reflux. After 16 h the reaction was allowed to cool and concentrated in vacuo to give crude title compound. MS: (ES (M+1)), 191.

### d) [7-(1-Methyl-1H-imidazol-2-yl)[1,2,4]triazolo[4,3-b]pyridazin-3-yl]-(4-trifluoromethyl-phenyl)-amine

To the product of step c) (assume 3.84 mmol) in acetonitrile was added 4-trifluoromethylphenylisocyanate (718 mg, 3.84 mmol). The white slurry was stirred for 24 h. Mass spectrometry showed (MH⁺) 378. Phosphorus oxychloride (0.71 ml, 7.86 mmol) was then added and the mixture heated to 90° C. After 4 days the green solution was allowed to cool to room temperature, then quenched by addition of saturated NaHCO₃. The mixture was extracted three times with ethyl acetate and dried over sodium sulfate. After filtration a portion of the crude material was purified by mass triggered HPLC (Nebula) to afford the title compound (28.5 mg) as a yellow solid.

¹H NMR (400 MHz, DMSO) δ 10.29 (1H, s) 8.96 (1H, d, J=1.8 Hz), 8.46 (1H, d, J=1.8 Hz), 8.06 (2H, d, J=8.3 Hz), 7.69 (2H, d, J=8.3 Hz), 7.44 (1H, s), 7.15 (1H, s), 3.96 (3H, s) ppm; MS: (ES (M+1)), 360.

### EXAMPLE 116

### N-[4-Trifluoromethylphenyl]-7-[3-trifluoromethylpyridin-2-yl]imidazo[1,2a]pyridin-3-amine

Description 43 (140 mg, 0.45 mmol) was dissolved in dioxane (5 ml) and hydrogenated at atmospheric pressure over 10% Pd/C at room temperature overnight. The mixture was filtered through a syringe filter directly into a 5 ml microwave tube containing caesium carbonate (207 mg, 0.63 mmol), 9,9-dimethyl-4,5-bis(diphenylphosphino)xanthene [xantphos] (16 mg, 0.027 mmol) and tris(dibenzylideneacetone)dipalladium (0) (8.3 mg, 0.009 mmol) under nitrogen. 4-Bromobenzotrifluoride (0.063 ml, 0.45 mmol) was added and the resulting mixture was irradiated for 20 min at 170° C. in a Smith Personal Chemistry® microwave. The mixture was concentrated under reduced pressure and purified by flash chromatography (Biotage 25S®, 2% MeOH/DCM). Further purification by mass-triggered HPLC followed by filtration through an SCX cartridge, followed by trituration (diethyl ether/isohexane) gave a pale orange solid (20 mg, 10%).

MS: (ES (M+1)) 423; 1H NMR δ (ppm)(DMSO): 6.74 (2H, d, J=8.5 Hz), 7.08 (1H, d, J=7.1 Hz), 7.52 (2H, d, J=8.5 Hz), 7.70 (3H, m), 8.11 (1H, d, J=7.1 Hz), 8.37 (1H, d, J=6.8 Hz), 8.70 (1H, s), 8.95 (1H, d, J=3.9 Hz).

### EXAMPLE 117

### N-[4-trifluoromethylphenyl]-2-[3-trifluoromethylpyridin-2-yl]imidazo[1,5-a]pyrimidin-6-amine

Prepared from Description 45 according to the procedure of Example 82 MS: (ES (M+1)) 424. ¹H NMR (400 MHz, DMSO) δ 9.57 (1H, s), 8.96 (1H, dd, J=1.0, 4.7 Hz), 8.62 (1H, dd, J=1.0, 7.7 Hz), 8.40 (1H, dd, J=1.4, 8.1 Hz), 7.75 (1 H, dd, J=4.7, 7.7 Hz), 7.63 (4H, s), 7.49 (1H, d, J=1.0 Hz), 7.18 (1H, d, J=7.7 Hz).

### EXAMPLE 118

### N-(4-trifluoromethylphenyl)-7-(2-methoxyphenyl) [1,2,4]triazolo[4,3-b]pyridazine-3-amine

Using a procedure analogous to Example 1 but starting with Description 3 the title compound was obtained (0.16 g, 12%) as a bright yellow solid, MS: (ES (M+1)) 386.

¹H NMR (500 MHz, DMSO) δ 3.87 (3H, s), 7.14 (1H, tr., J=6.7), 7.23 (1H, d, J=7.8), 7.50 (1H, tr., J=7.3), 7.58 (1H, d, J=6.7), 7.69 (2H, d, J=7.8), 8.04 (2H, d, J=7.8), 8.26 (1H, d, J=1.6), 8.75 (1H, d, J=1.6), 10.19 (1H, s) ppm.

### EXAMPLE 119

### 4-Trifluoromethylphenyl-3-(3-trifluoromethylpyridin-2-yl)imidazo[1,5-b]pyridazin-7-ylamine hydrochloride

Hydrochloric acid (2N, 1.3 eq) was added to a slurry of Example 75 (40 mg, 0.094 mmol) in ethanol (2.5 ml) and the mixture swirled with gentle warming until the solid dissolved. The solution was evaporated to dryness and azeotroped with ethanol to give the title compound as an orange solid (40 mg). m/z (ES⁺) 424 (M+H⁺). ¹H NMR (400 MHz, MeOH-d₄) 8.95 (1H, d, J 4.9), 8.54 (1H, d, J 2.1), 8.36 (1H, d, J 7.8), 8.17 (1H, d, J 1.9), 7.78-7.72 (3H, m), 7.63 (3×, m).

### Biological Methodology

Determination of In Vitro Activity

CHO cells, stably expressing recombinant human VR1 receptors and plated into black-sided 384-well plates, were washed twice with assay buffer (Hepes-buffered saline) and then incubated with 1 uM Fluo-3-AM for 60 minutes in darkness. Cells were washed twice more to remove excess dye, before being placed, along with plates containing capsaicin and test compounds in a Molecular Devices FLIPR. The FLIPR simultaneously performed automated pharmacological additions and recorded fluorescence emmission from Fluo-3. In all experiments, basal fluorescence was recorded, before addition of test compounds and subsequent addition of a previously determined concentration of capsaicin that evoked 80% of the maximum respsonse. Inhibition of capsaicin evoked increases in intracellular [Ca²⁺] were expressed relative to wells on the same plate to which capcaicin was added in the absence of test compounds. Increases in intracellular [Ca²⁺] occuring after addition of test compound alone, prior to addition of capsaicin, allow determination of intrinsic agonist or partial agonist activity, if present. All the above compounds had an IC₅₀ of below 2M.

Determination of In Vivo Efficacy in a Capsaicin Paw Flinch Model

(Method adapted from Taniguchi et al, 1997, Br J Pharmacol 122(6):809-12) To determine in vivo functional occupancy of VR1 receptors, compounds are administered orally to male Sprague Dawley rats typically 1 hour prior to receiving an intraplantar injection of capsaicin (2 Tg dissolved in ethanol) and the number of flinches of the injected paw is recorded for 5 minutes immediately thereafter. Statistical analysis is performed using one-way ANOVA followed by Dunnett's test; p values <0.05 compared to capsaicin/vehicle-treated rats are considered significant.

Determination of In Vivo Efficacy in a Model of Inflammatory Pain

(Method adapted from Hargreaves et al., 1988 Pain, 32(1):77-88).

Antinociceptive activity is determined using a rat carrageenan-induced thermal hyperalgesia assay. Inflammatory hyperalgesia is induced by intraplantar injection of carrageenan (lambda-carrageenan 0.1 ml of 1% solution made up in saline) into one hind paw. Compounds are given orally typically 2 hours after carrageenan and paw withdrawal latancies determined 1 hour later. Paw withdrawal latencies to application of noxious thermal stimuli to plantar surface of the hind paw are measured using the Hargreaves apparatus. Thermal hyperalgesia is defined as the difference in paw withdrawal latencies for saline/vehicle- and carrageenan/vehicle-treated rats. Paw withdrawal latencies for drug treated rats are expressed as a percentage of this response. Statistical analysis is performed using one-way ANOVA followed by Dunnett's test; p values <0.05 compared to carrageenan/vehicle-treated rats are considered significant.

1. A compound of formula 1: 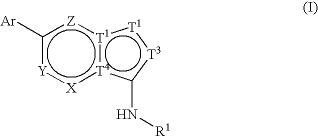
wherein:
one of T¹ and T⁴ is N and the other is C;
T² and T³ are independently N or C(CH₂)ₙR²;
X, Y and Z are independently N or C(CH₂)ₙR³;
R¹ is Ar¹ or R¹ is C₁₋₆alkyl optionally substituted with one or two groups Ar¹;
Ar¹ is cyclohexyl, piperidinyl, piperazinyl, morpholinyl, adamantyl, phenyl, naphthyl, a six-membered heteroaromatic ring containing one, two or three nitrogen atoms, a five-membered heteroaromatic ring containing one, two, three or four heteroatoms chosen from O, N and S, at most one O or S atom being present, or a nine- or ten-membered bicyclic heteroaromatic ring in which phenyl or a six-membered heteroaromatic ring as defined above is fused to a six- or five-membered heteroaromatic ring as defined above;
Ar¹ is optionally substituted by one, two or three groups chosen from halogen, hydroxy, cyano, nitro, isonitrile, CF₃, OCF₃, SF₅, C₁₋₆alkyl, C₂₋₆alkenyl, C₂₋₆alkynyl, C₁₋₆alkoxy, C₁₋₆alkylthio, C₁₋₆alkylsulfinyl, C₁₋₆alkylsulfonyl, —NR⁶R⁷, CONR⁶R⁷, —COH, —C₁₂H, C₁₋₆alkylcarbonyl, C₁₋₆alkoxycarbonyl, haloC₁₋₆alkyl, haloC₂₋₆alkenyl, hydroxyC₁₋₆alkyl, aminoC₁₋₆alkyl, cyanoC₁₋₆alkyl, C₃₋₆cycloalkyl, hydroxyC₃₋₆cycloalkyl, aminoC₃₋₆cycloalkyl, haloC₃₋₆cycloalkyl, cyanoC₃₋₆cycloalkyl, haloC₁₋₆alkylcarbonyl, C₁₋₆alkoxycarbonylC₁₋₆alkyl, (halo)(hydroxy)C₁₋₆alkyl, (halo)(hydroxy)C₃₋₆cycloalkyl, phenyl and a five-membered heteroaromatic ring containing one, two or three heteroatoms, at most one O or S atom being present; wherein the phenyl and five-membered heteroaromatic ring are optionally substituted by C₁₋₆alkyl, halo, hydroxy or cyano; when two C₁₋₆alkyl groups substitute adjacent positions on Ar¹ then, together with the carbon atoms to which they are attached, they may form a partially saturated ring containing five or six carbon atoms; when two C₁₋₆alkoxy groups substitute adjacent positions on Ar¹ then, together with the carbon atoms to which they are attached, they may form a partially saturated five- or six-membered ring;
Ar is phenyl, a six-membered heteroaromatic ring containing one, two or three nitrogen atoms or a five-membered heteroaromatic ring containing one, two, three or four heteroatoms chosen from O, N and S, at most one heteroatom being O or S, Ar being optionally substituted by one, two or three groups chosen from halogen, CF₃, OCF₃, C₁₋₆alkyl, C₂₋₆alkenyl, C₂₋₆alkynyl, nitro, cyano, isonitrile, hydroxy, C₁₋₆alkoxy, C₁₋₆alkylthio, —NR⁶R⁷, —CONR⁶R⁷, —COH, CO₂H, C₁₋₆alkoxycarbonyl, haloC₁₋₆alkyl, hydroxyC₁₋₆alkyl, aminoC₁₋₆alkyl, C₁₋₆alkylcarbonyl and a five-membered heteroaromatic ring containing one, two, three or four heteroatoms chosen from O, N and S, at most one heteroatom being O or S, optionally substituted by C₁₋₆alkyl, halogen, amino, hydroxy or cyano;
R² and R³ are independently hydrogen, halogen, CF₃, OCF₃, C₁₋₆alkyl, C₂₋₆alkenyl, C₂₋₆alkynyl, nitro, cyano, isonitrile, hydroxy, C₁₋₆alkoxy, C₁₋₆alkylthio, —NR⁶R⁷, —CONR⁶R⁷, —COH, CO₂H, C₁₋₆alkoxycarbonyl, haloC₁₋₆alkyl, hydroxyC₁₋₆alkyl, aminoC₁₋₆alkyl, C₁₋₆alkylaminoC₁₋₆alkyl, di(C₁₋₆alkyl)aminoC₁₋₆alkyl, amido, piperidinyl, piperazinyl, C₃₋₆cycloalkyl, morpholinyl, phenyl, a six-membered heteroaromatic ring containing one, two or three nitrogen atoms or a five-membered heteroaromatic ring containing one, two, three or four heteroatoms chosen from O, N and S, at most one O or S atom being present, which phenyl, six-membered heteroaromatic ring and five-membered heteraromatic ring are optionally substituted by haloC₁₋₆alkyl, C₁₋₆alkyl, hydroxy, halogen, amino or cyano;
R⁶ and R⁷ are independently hydrogen or C₁₋₆alkyl; when both R⁶ and R⁷ are C₁₋₆alkyl then, together with the nitrogen atom to which they are attached, they may form a five or six membered saturated nitrogen containing ring; and
n is zero, one, two or three;
or a pharmaceutically acceptable salt thereof.

2. A compound according to claim 1 in which R¹ is phenyl, pyridyl, piperidinyl, butyl, adamantyl or cyclohexyl.

3. A compound according to claim 1 or 2 in which Ar¹ is substituted by halogen, hydroxy, cyano, CF₃, SF₅, OCF₃, C₁₋₄alkyl, C₂₋₄alkenyl, C₂₋₄alkynyl, C₁₋₄alkoxy, C₁₋₄alkylthio, C₁₋₄alkylsulfinyl, C₁₋₄alkylsulfonyl, —NR⁶R⁷, cyanoC₁₋₄alkyl, haloC₁₋₄alkylcarbonyl, C₁₋₄alkylcarbonyl, C₁₋₄alkoxycarbonyl, haloC₁₋₄alkyl, haloC₂₋₄alkenyl, hydroxyC₁₋₄alkyl, C₃₋₆cycloalkyl, cyanoC₃₋₆cycloalkyl, (halo)(hydroxy)C₁₋₄alkyl, C₁₋₄alkoxycarbonylC₁₋₄alkyl, phenyl, or a five-membered heteroaromatic ring as defined above where the phenyl or five-membered heteroaromatic ring is unsubstituted or substituted by C₁₋₄alkyl or halogen.

4. A compound according to claim 1, 2 or 3 in which Ar is phenyl or a 5- or 6-membered heteroaromatic ring containing one or two nitrogen atoms.

5. A compound according to any preceding claim in which Ar is monosubstituted ortho to the point of attachment to the rest of the molecule.

6. A compound according to claim 1 which is:
N-(4-trifluoromethylphenyl)-7-(3-trifluoromethyl-2-pyridyl)[1,2,4]triazolo[4,3-b]pyridazine-3-amine;
N-(4-tert-butyl)phenyl)-7-(3-trifluoromethyl-2-pyridyl)[1,2,4]triazolo[4,3-b]pyridazine-3-amine;
N-phenyl-7-[3-trifluoromethyl-2-pyridinyl][1,2,4]triazolo[4,3-b]pyridazin-3-amine;
N-[2-trifluoromethylphenyl]-7-[3-trifluoromethyl-2-pyridinyl][1,2,4]triazolo[4,3-b]pyridazin-3-amine;
N-(3-chlorophenyl)-7-[3-trifluoromethyl-2-pyridinyl][1,2,4]triazolo[4,3-b]pyridazin-3-amine;
N-[3-trifluoromethylphenyl]-7-[3-trifluoromethyl-2-pyridinyl][1,2,4]triazolo[4,3-b]pyridazin-3-amine;
N-(2,4-difluorophenyl)-7-[3-trifluoromethyl-2-pyridinyl][1,2,4]triazolo[4,3-b]pyridazin-3-amine;
N-[4-methoxyphenyl]-7-[3-trifluoromethyl-2-pyridinyl][1,2,4]triazolo[4,3-b]pyridazin-3-amine;
N-[2-(1-methylethyl)phenyl]-7-[3-trifluoromethyl-2-pyridinyl][1,2,4]triazolo[4,3-b]pyridazin-3-amine;
N-[3-methylsulfanylphenyl]-7-[3-trifluoromethyl-2-pyridinyl][1,2,4]triazolo[4,3-b]pyridazin-3-amine;
N-(2-naphthalenyl)-7-[3-trifluoromethyl-2-pyridinyl][1,2,4]triazolo[4,3-b]pyridazin-3-amine;
N-{4-trifluoromethoxyphenyl}-7-[3-trifluoromethyl-2-pyridinyl][1,2,4]triazolo[4,3-b]pyridazin-3-amine;
N-(2-phenylethyl)-7-[3-trifluoromethyl-pyridinyl][1,2,4]triazolo[4,3-b]pyridazin-3-amine;
N-(1,3-benzodioxol-5-yl)-7-[3-trifluoromethyl-2-pyridinyl][1,2,4]triazolo[4,3-b]pyridazin-3-amine;
N-[3-fluorophenylmethyl]-7-[3-trifluoromethyl-2-pyridinyl][1,2,4]triazolo[4,3-b]pyridazin-3-amine;
2-({7-[3-trifluoromethyl-2-pyridinyl][1,2,4]triazolo[4,3-b]pyridazin-3-yl}amino)benzonitrile;
N-(diphenylmethyl)-7-[3-trifluoromethyl-2-pyridinyl][1,2,4]triazolo[4,3-b]pyridazin-3-amine;
N-[(1S)-1-phenylethyl]-7-[3-trifluoromethyl-2-pyridinyl][1,2,4]triazolo[4,3-b]pyridazin-3-amine;
N-(2,4-dichlorophenyl)-7-[3-trifluoromethyl-2-pyridinyl][1,2,4]triazolo[4,3-b]pyridazin-3-amine;
N-(3,4-dichlorophenyl)-7-[3-trifluoromethyl-2-pyridinyl][1,2,4]triazolo[4,3-b]pyridazin-3-amine;
N-[4-dimethylaminophenyl]-N-{7-[3-trifluoromethyl-2-pyridinyl][1,2,4]triazolo[4,3-b]pyridazin-3-yl}amine;
N-[(3,4-dichlorophenyl)methyl]-7-[3-trifluoromethyl-2-pyridinyl][1,2,4]triazolo[4,3-b]pyridazin-3-amine;
N-(4-chloro-2-methylphenyl)-7-[3-trifluoromethyl-2-pyridinyl][1,2,4]triazolo[4,3-b]pyridazin-3-amine;
N-(3-chloro-4-fluorophenyl)-7-[3-trifluoromethyl-2-pyridinyl][1,2,4]triazolo[4,3-b]pyridazin-3-amine;
N-[2-fluoro-6-trifluoromethylphenyl]-7-[3-trifluoromethyl-2-pyridinyl][1,2,4]triazolo[4,3-b]pyridazin-3-amine;
N-[4-fluoro-2-trifluoromethylphenyl]-7-[3-trifluoromethyl-2-pyridinyl][1,2,4]triazolo[4,3-b]pyridazin-3-amine;
N-[4-fluoro-3-trifluoromethylphenyl]-7-[3-trifluoromethyl-2-pyridinyl][1,2,4]triazolo[4,3-b]pyridazin-3-amine;
N-[2-chloro-4-trifluoromethylphenyl]-7-[3-trifluoromethyl-2-pyridinyl][1,2,4]triazolo[4,3-b]pyridazin-3-amine;
N-(2,3-dihydro-1H-inden-5-yl)-7-[3-trifluoromethyl-2-pyridinyl][1,2,4]triazolo[4,3-b]pyridazin-3-amine;
N-(2,3-dihydro-1,4-benzodioxin-6-yl)-7-[3-trifluoromethyl-2-pyridinyl][1,2,4]triazolo[4,3-b]pyridazin-3-amine;
N-(4-trifluoromethylphenyl)-7-(3-methyl-2-pyridyl)[1,2,4]triazolo[4,3-b]pyridazine-3-amine;
5-chloro-7-(3-methyl-2-pyridyl)-N-(4-trifluoromethylphenyl)[1,2,4]triazolo[4,3-a]pyridin-3-amine;
5-chloro-7-(2-methoxyphenyl)-N-(4-trifluoromethylphenyl)[1,2,4]triazolo[4,3-a]pyridin-3-amine;
5-chloro-N-(4-trifluoromethylphenyl)-7-(3-trifluoromethyl-2-pyridyl)[1,2,4]triazolo[4,3-a]pyridin-3-amine;
6-chloro-N-(5-isoquinolyl)-7-(4-trifluoromethylphenyl)[1,2,4]triazolo[4,3-b]pyridazin-3-amine; 7-(3-methyl-2-pyridyl)-N-(4-trifluoromethylphenyl)[1,2,4]triazolo[4,3-a]pyridin-3-amine;
7-(3-trifluoromethyl-2-pyridyl)-N-(4-trifluoromethylphenyl)[1,2,4]triazolo[4,3-a]pyridin-3-amine;
7-(2-methoxyphenyl)-N-(4-trifluoromethylphenyl)[1,2,4]triazolo[4,3-a]pyridin-3-amine;
N-(5-isoquinolyl)-7-(4-trifluoromethylphenyl)[1,2,4]triazolo[4,3-b]pyridazine-3-amine;
7-(3-trifluoromethyl-2-pyridyl)-N-(5-trifluoromethyl-2-pyridyl)[1,2,4]triazolo[4,3-b]pyridazine-3-amine;
N-(4-trifluoromethylphenyl)-6-(3-trifluoromethylpyrid-2-yl)pyrazolo[1,5-a]pyrimidin-3-amine;
4-trifluoromethylphenyl-3-(3-trifluoromethylpyridin-2-yl)imidazo[1,5-b]pyridazin-7-ylamine;
N-[4-trifluoromethylphenyl]-7-[3-trifluoromethylpyridin-2-yl]imidazo[1,2-b]pyridazin-3-amine;
7-[3-trifluoromethylpyridin-2-yl]-N-[5-trifluoromethylpyridin-2-yl]imidazo[1,2-b]pyridazin-3-amine;
N-(5-methylpyridin-2-yl)-7-[3-trifluoromethylpyridin-2-yl]imidazo[1,2-b]pyridazin-3-amine;
[7-(3-methylpyridin-2-yl)-[1,2,4]triazolo[4,3-b][1,2,4]triazin-3-yl]-(4-trifluoromethylphenyl)amine; and
[7-(1-methyl-1H-imidazol-2-yl)[1,2,4]triazolo[4,3-b]pyridazin-3-yl]-(4-trifluoromethylphenyl)amine;
or a pharmaceutically acceptable salt thereof.

7. A pharmaceutical composition comprising a compound according to claim 1, or a pharmaceutically acceptable salt thereof, and a pharmaceutically acceptable carrier.

8. A compound according to claim 1, or a pharmaceutically acceptable salt thereof, for use in a method of treatment of the human or animal body by therapy.

9. (canceled)

10. A method of treatment of a subject suffering from pain which comprises administering to that subject a therapeutically effective amount of compound according to claim 1, or a pharmaceutically acceptable salt thereof.
